(19) United States
(12) Patent Application Publication
(10) Pub. No.: US 2026/0363650 A1
(43) Pub. Date: Nov. 19, 2026

(54) IMPLANTABLE FLUID FLOW AND ACOUSTIC SENSOR

(71) Applicant: CANARY MEDICAL SWITZERLAND AG, Baar (CH)

(72) Inventors: Peter J. Schiller, Shakopee, MN (US); Jeffrey M. Gross, Carlsbad, CA (US); Mark A. Adler, Carlsbad, CA (US); Kimberly Salant, Asbury, NJ (US); Heidi Sharipov, San Diego, CA (US)

(21) Appl. No.: 19/144,731

(22) PCT Filed: Jan. 5, 2024
(86) PCT No.: PCT/US2024/010540

§ 371 Date: Jun. 30, 2025

Related U.S. Application Data
(60) Provisional application No. 63/437,571, filed on Jan. 6, 2023.
(60) Provisional application No. 63/540,800, filed on Sep. 27, 2023.

(51) Int. Cl. A61B 8/06; A61B 8/00; A61B 8/02; B06B 1/06

(52) U.S. Cl. CPC A61B 8/06; A61B 8/02; A61B 8/4281; A61B 8/56; B06B 1/0651; A61B 8/488; B06B 2201/76

(57) ABSTRACT

Disclosed are devices, systems, and methods for in vivo monitoring of the flow of a biological fluid in an anatomic structure, such as blood flow through a heart valve (e.g., mitral valve, tricuspid valve, and/or aortic valve) or heart chamber, and an acoustic signal that emanates from an internal body structure. The disclosed devices, systems, and methods can continuously and autonomously measure and evaluate cardiovascular and pulmonary function or dysfunction and/or in compilation with other monitored data that can be used to measure or predict secondary factors.

### CROSS-REFERENCE TO RELATED APPLICATIONS

All applications for which a foreign or domestic priority claim is identified in the Application Data Sheet as filed with the present application are hereby incorporated by reference.

### TECHNICAL FIELD

This present disclosure relates generally to implantable medical devices and more specifically to sensors deployable in the body of a patient user for monitoring fluid flow and acoustic signals associated with biological function in vivo.

### BACKGROUND

According to the World Health Organization (WHO), cardiovascular diseases are among the leading causes of death globally, resulting in an estimated 17.9 million lives lost each year. In the United States, heart disease is the leading cause of death, with coronary artery disease (CAD) being the most pernicious class by causing decreased blood flow to the heart that often leads to a heart attack. CAD and other heart valve diseases, like mitral valve prolapse (MVP) and mitral valve regurgitation (MVR), are silent killers because persons typically do not know the existence of their underlying heart problems and therefore are not diagnosed until the person experiences signs or symptoms of a heart attack, heart failure, or an arrhythmia-which may be too late to survive or recover with a reasonable quality of life. Even now, a person must typically have a secondary medical condition, such as diabetes or obesity, or be identified with one or more significant risk factors, such as high blood pressure, high cholesterol, high alcohol use, or smoking, for the physician to initiate a round of testing and remote monitoring of the person's heart. This is because there is not an accessible, uncomplicated, and affordable device or technique to observe and track a person's heart function.

Presently, examination of heart function to potentially diagnose heart disease is limited to the clinical environment. Typically, a patient would undergo a variety of different tests, including analyte testing from blood samples; imaging such as a chest X-ray, CT-scan, or magnetic resonance imaging (MRI) of the heart; and physiological signal monitoring, such as an electrocardiogram (ECG or EKG), which are electrical signal recordings of the heart that can indicate heart rate and detect irregular heartbeats when monitored remotely (e.g., by a Holter monitor), or an echocardiogram, which is a noninvasive acoustic (sound) signal monitoring technique used to create images of the heart and blood in motion. Moreover, patients are only prescribed such tests after experiencing an acute or emergency medical treatment, such as cardiac arrest, stroke, severe dizziness or unconsciousness, or extreme chest pain.

To date, there have been some advancements in remote monitoring to begin turning the tide against heart disease. For instance, wearable heart rate monitors are becoming a ubiquitous option for tracking heart rate during exercise or daily activity, monitoring stress and movement levels, tracking sleep habits at night, and in some versions of these wearable devices, testing certain vital signs outside of the clinic, such as ECG to determine singular heart rhythm events, such as a healthy sinus rhythm or risky atrial fibrillation. While these devices are well suited to promote healthier active lifestyles that may contribute to preventing the onset of heart disease or mitigating minor-to-moderate heart disease in the long run, they are ill equipped to identify symptoms or hallmarks of the vast array of heart diseases and incapable of determining any underlying biological or physiological factors at the root of heart disease.

The challenge for clinicians is to catch heart disease early or manage and monitor diagnosed heart disease post-treatment. Yet, monitoring heart function relies on patient participation and compliance; and existing systems and methods are too time intensive, costly, and under-resourced to be effective.

There is a need for a new paradigm of sensors deployable in the body of a patient user and capable of monitoring the dynamic flow of biological fluids in vivo, such as blood flow across a valve of the heart, to characterize overall heart function.

### SUMMARY

In brief, disclosed are devices, systems, and methods for in vivo monitoring of both the flow of a biological fluid in an anatomic structure, such as blood flow across a heart valve or blood flow through a heart chamber or vessel flowing blood into or out of the heart, and the acoustic signals associated with physiological phenomena of an internal body structure, such as the heart and/or lungs, all from within the host. The disclosed devices, systems, and methods include an in vivo fluid flow sensor and an in vivo acoustic sensor, which can be configured as part of a single device structure or separate device structures implanted in the host's body.

In some embodiments in accordance with the present technology, an implantable medical device includes an in vivo fluid flow sensor and an in vivo acoustic sensor. For example, in some implementations, the in vivo fluid flow sensor is configured to transmit an ultrasound signal to propagate through an anatomic structure and to detect ultrasound signals that have propagated through the anatomic structure and are indicative of a fluid flow of a biological fluid in the anatomic structure; and the in vivo acoustic sensor is configured to detect an acoustic signal emanating from an internal body structure. For example, in some embodiments, the in vivo fluid flow sensor comprises a linkage assembly comprising a first arm configured to attach to a first portion of the anatomic structure and a second arm configured to attach to a second portion of the anatomic structure opposite to the first portion, and an ultrasound sensor assembly comprising a plurality of ultrasound transducer elements coupled to the linkage assembly; and the in vivo acoustic sensor comprises a hermetically sealed housing, and a transducer element configured to receive the acoustic signal that emanates from the internal body structure such that the transducer element converts energy of the received acoustic signal to an electrical signal indicative of a physiological function by the internal body structure.

In some embodiments in accordance with the present technology, an implantable medical device for in vivo monitoring of an anatomic structure from within a host's body includes (i) an in vivo fluid flow sensor, which comprises a linkage assembly comprising a first arm configured to attach to a first portion of the anatomic structure and a second arm configured to attach to a second portion of the anatomic structure opposite to the first portion, a connection apparatus coupled to each of the first arm and the second arm, an ultrasound sensor assembly comprising a plurality of ultrasound transducer elements coupled to the linkage assembly, the plurality of ultrasound transducer elements including a first ultrasound transducer element that is configured to transmit an ultrasound signal to propagate through the anatomic structure and a second ultrasound transducer element and a third ultrasound transducer element that are configured to receive ultrasound signals that have propagated through the anatomic structure and are indicative of a fluid flow parameter of a biological fluid in the anatomic structure; (ii) an in vivo acoustic sensor, which comprises a hermetically sealed housing, and a transducer element configured to receive an acoustic signal that emanates from a source within the host's body such that the transducer element converts energy of the received acoustic signal to an electrical signal indicative of a physiological function by the source within the host's body; and (iii) an electronics unit at least partially housed in the connection apparatus of the in vivo fluid flow sensor and/or at least partially housed in the hermetically sealed housing of the in vivo fluid flow sensor, wherein the electronics unit is in electrical communication with the plurality of ultrasound transducer elements of the ultrasound sensor assembly and in electrical communication with the transducer element of the in vivo acoustic sensor, the electronics unit configured to process electrical signals associated with the received ultrasound signals and the received acoustic signal as data and wirelessly transmit the data to an external processor.

In some embodiments in accordance with the present technology, a system for in vivo monitoring of fluid flow in an anatomic structure includes an implantable medical device including an in vivo fluid flow sensor and an in vivo acoustic sensor; and a data processing system, comprising a processor and a memory, in data communication with the implantable medical device and configured to receive the data from the implantable medical device and process the received data to determine a fluid flow parameter associated with a biological fluid in the anatomic structure and/or an acoustic signal parameter associated with a physiological function of an internal body structure.

The above-mentioned and additional features of the present technology and the manner of obtaining them will become apparent, and the disclosed technology may be best understood by reference to the following more detailed description. It is noted that all references disclosed herein are hereby incorporated by reference in their entirety as if each was incorporated individually.

This Summary has been provided to introduce certain concepts in a simplified form that are further described in detail below in the Detailed Description. Except where otherwise expressly stated, this Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Many aspects of the present disclosure can be better understood with reference to the following drawings. The features in the drawings are not necessarily to scale, fully shown, or depicted in the same manner as would be physically constructed. Instead, emphasis is placed on clearly illustrating the principles of the present disclosure. The drawings should not be taken to limit the disclosure to the specific embodiments depicted but are for explanation and understanding only.

FIG. 1A shows a diagram illustrating an example embodiment of a system for in vivo monitoring of biological fluid flow and acoustic signals from within a patient having an implantable medical device (IMD), in accordance with the present technology, for monitoring, analyzing, and reporting events associated with the cardiovascular and/or pulmonary health of a user, in accordance with the present technology.

FIG. 1B shows a block diagram illustrating an example embodiment of an in vivo fluid flow sensor device of FIG. 1A, in accordance with the present technology.

FIG. 1C shows a block diagram of an example embodiment of an in vivo acoustic sensor device of FIG. 1A, in accordance with the present technology.

FIG. 2A shows a diagram depicting an example embodiment of the in vivo fluid flow sensor device of FIG. 1B, in accordance with the present technology.

FIG. 2B shows a diagram depicting another example embodiment of the in vivo fluid flow sensor device of FIG. 1B including a second set of ultrasound sensor contingents, in accordance with the present technology.

FIG. 2C shows a diagram depicting another example embodiment of the in vivo fluid flow sensor device of FIG. 1B, in accordance with the present technology.

FIG. 2D shows a diagram depicting another example embodiment of the in vivo fluid flow sensor device of FIG. 1B, in accordance with the present technology.

FIG. 3 shows a diagram illustrating an example embodiment of the in vivo fluid flow sensor device of FIG. 2A attached to a heart of a patient user, in accordance with the present technology.

FIG. 4A shows a diagram depicting insertion sites for implanting an in vivo fluid flow sensor device in accordance with the present technology.

FIG. 4B shows diagrams illustrating example shape conformations of example embodiments of the in vivo fluid flow sensor device, in accordance with the present technology, for an implantation process near a target anatomic structure and a deployment process to secure to the target anatomic structure.

FIG. 5A shows a diagram depicting an example embodiment of a single-sided acoustic transducer configuration for an in vivo fluid flow sensor device in accordance with the present technology.

FIG. 5B shows a diagram depicting another example arrangement of a single-sided acoustic transducer configuration on an array for an in vivo fluid flow sensor device in accordance with the present technology.

FIG. 5C shows a diagram depicting another example embodiment of a single-sided acoustic transducer configuration with a reflector for an in vivo fluid flow sensor device in accordance with the present technology.

FIG. 5D shows a diagram depicting an example embodiment of a double-sided acoustic transducer configuration for an in vivo fluid flow sensor device in accordance with the present technology.

FIGS. 6A, 6B and 6C show diagrams depicting other example embodiments of the in vivo fluid flow sensor device of FIG. 1B, in accordance with the present technology.

FIG. 6D shows an exploded diagram depicting an example embodiment of an electronics unit and housing of the in vivo fluid flow sensor devices of FIGS. 6A, 6B and 6C.

FIG. 7A shows a diagram depicting an example embodiment of the in vivo fluid flow sensor device of FIG. 1B, in accordance with the present technology.

FIGS. 7B and 7C show diagrams depicting an example embodiment of the in vivo fluid flow sensor device of FIG. 1B, in accordance with the present technology.

FIG. 8 shows a diagram depicting another example embodiment of the in vivo fluid flow sensor device of FIG. 1B, in accordance with the present technology.

FIG. 9 shows a diagram of an example embodiment of a remote in vivo device that can tether to example embodiment of the in vivo fluid flow sensor device of FIG. 1B, such as the in vivo fluid flow sensor device of FIGS. 7A, 7B, 7C, and/or 8.

FIGS. 10A and 10B show diagrams illustrating an example embodiment of the in vivo fluid flow sensor device of FIG. 1B attached to a heart of a patient user in an example implementation of the device.

FIG. 11A shows a diagram depicting an example embodiment of the in vivo acoustic sensor device of FIG. 1C, shown in a portion of an exemplary IMD of the present technology.

FIG. 11B shows a cross-sectional view depicting an enlarged portion of the diagram of FIG. 11A with the membrane of the in vivo acoustic sensor in a relaxed state.

FIG. 11C shows a cross-sectional view depicting an enlarged portion of the image of FIG. 11A with the membrane of the in vivo acoustic sensor in a deflected state.

FIGS. 12A and 12B show a diagram depicting an example embodiment of the in vivo acoustic sensor device of FIG. 1C configured as an in vivo microphone in an exemplary IMD of the present technology, including a strain gauge and/or a piezoelectric element.

FIGS. 13A and 13B show a diagram depicting an example embodiment of the in vivo acoustic sensor device of FIG. 1C configured as an in vivo microphone in an exemplary IMD of the present technology, including a capacitive electrode sensor and/or a sensor with electret condenser elements.

FIG. 14A shows a top view of a diagram depicting an example embodiment of the in vivo acoustic sensor device of FIG. 1C in an IMD, showing an example form factor configuration of the IMD including a battery, an electronics package, and an antenna.

FIG. 14B shows a side view diagram of the exemplary IMD of FIG. 14A.

FIG. 15A shows a top view of a diagram depicting an example embodiment of the in vivo acoustic sensor device of FIG. 1C in an IMD, showing an example form factor configuration of the IMD including a battery, an electronics package with a deflective membrane, and an antenna.

FIG. 15B shows a side view diagram of the exemplary IMD of FIG. 15A.

FIG. 16A shows a top view of a diagram depicting an example embodiment of the in vivo acoustic sensor device of FIG. 1C in an IMD, showing an example form factor configuration of the IMD including a battery, an electronics package and an antenna, where an accelerometer is contained within a tethered component.

FIG. 16B shows a side view diagram of the exemplary IMD of FIG. 16A but omitting the tethered component.

FIG. 17 shows a block diagram depicting an exemplary IMD of the present technology, including internal components of the IMD corresponding to FIG. 14A and FIG. 14B and/or FIG. 15A and FIG. 15B.

FIG. 18 shows a block diagram depicting an exemplary IMD of the present technology having a tethered component including an in vivo acoustic sensor device comprising a high fidelity (HF) accelerometer and including internal components of the IMD corresponding to FIG. 16A and FIG. 16B.

FIG. 19 shows a block diagram depicting an exemplary IMD of the present technology having a microphone included in an in vivo acoustic sensor device.

FIGS. 20A and 20B show diagrams depicting an example embodiment of an IMD of the present technology having a stress-mediated acoustic sensor, in accordance with the present technology, hermetically sealed with an electronics unit within a housing.

FIGS. 21A, 21B and 21C show diagrams depicting example embodiments of a unimorph piezoelectric sensor device, in accordance with the present technology.

FIG. 22 shows diagrams depicting example geometries of some example embodiments of a unimorph piezoelectric sensor device, in accordance with the present technology.

FIG. 23 shows a diagram illustrating an example embodiment of an implantable medical device in accordance with the present technology, integrating an example embodiment of an in vivo fluid flow sensor device and an example embodiment of the in vivo acoustic sensor device, in accordance with the present technology.

FIGS. 24A and 24B show diagram illustrating example embodiments of an implantable medical device in accordance with the present technology, interconnecting an example embodiment of an in vivo fluid flow sensor device and an example embodiment of the in vivo acoustic sensor device, in accordance with the present technology.

### DETAILED DESCRIPTION

The present technology provides a system for monitoring and collecting data from one or more implantable medical devices that is/are implanted into a living subject for assessment of one or more health and/or disease conditions. The living subject may be also referred to herein as a host, a patient, or simply a subject, which can include a human person or non-human animal. The IMD(s) of the present technology may be surgically implanted and removed from the host.

In some embodiments, for example, an IMD includes an in vivo fluid flow sensor that includes an ultrasound transducer array attachable to an outside of an anatomic structure, such as a heart chamber or blood vessel leading into or out of the heart or region of a lung or other organ, which can measure fluid flow through the anatomic structure. In some embodiments, for example, an IMD includes an in vivo acoustic sensor that can detect and measure an acoustic signal generated by the host. The IMD including the in vivo fluid flow sensor can also include the in vivo acoustic sensor, which can be configured as part of a single device structure (e.g., shared housing) or can be configured as part of separate device structures that can be electrically connected to share electronic components, e.g., including but not limited to a power supply, wireless communication unit, data processing unit, or other electronic resources and functionalities.

As used herein, an acoustic signal refers to mechanical waves in gases, liquids, and solids, including vibration, sound, ultrasound, and infrasound. Thus, the in vivo fluid flow sensor and the in vivo acoustic sensor can detect and measure mechanical waves that travel through the gases, liquids and/or solids that make up the internal anatomy of the host. Acoustic signals originating withing the body are generated by pressure changes, blood and air flow, and the mechanical motion of organs and tissues. Examples of the acoustic signals detectable by the in vivo acoustic sensor of the present technology can be turbulent flow of fluids, such as air flow in/out of the lungs or blood flow in structures of the heart or the vasculature, or the impulses from motion/movement of the heart structures (e.g., valves).

In some embodiments, the IMD including the in vivo fluid flow sensor and/or the in vivo acoustic sensor can include one or more auxiliary sensors to detect and measure motion and/or position or orientation of the patient, electrophysiological signals associated with one or more anatomic structures of the host, such as the heart, and sensors to detect blood velocity and vessel diameter, and/or an analyte or multiple analytes in an in vivo fluid of the patient. Example embodiments including the one or more auxiliary sensors with the in vivo fluid flow sensor and/or the in vivo acoustic sensor are discussed in detail below.

The term “sound” is commonly used to refer to audible mechanical waves (e.g., sounds waves) that can be detected by the human ear (i.e., heard by or audible to the human ear). While the IMDs of the present technology may detect and measure such sound waves, the IMDs may detect and measure mechanical waves that cannot be heard by the human ear. As used herein, and unless the context indicates otherwise, the terms “sound” and “acoustics” and “acoustic signals” and “mechanical waves” may be used interchangeably, without limiting the meaning of these terms to audible mechanical waves that can be heard by the human ear, i.e., mechanical waves in a 20 Hz to 20 kHz frequency range.

Some implementations of the disclosed devices, systems, and methods include in vivo monitoring of the flow of a biological fluid in an anatomic structure, such as blood flow through a heart valve (e.g., mitral valve, tricuspid valve, and/or aortic valve) or blood flow through a heart chamber, e.g., an atrium or ventricle, or a major blood vessel into or out of the heart, e.g., the aorta, vena cava, or pulmonary veins or arteries. The disclosed devices, systems, and methods can continuously measure and evaluate blood flow forward and backward at a region of the heart (e.g., an inlet or outlet of a heart valve) for determination of heart function or dysfunction (e.g., such as mitral valve regurgitation (MVR)) and/or in compilation with other monitored data that can be used to measure or predict secondary factors (e.g., such as heart rate (HR), cardiac output (CO), or hallmarks of congestive heart failure (CHF)).

Implementations of the disclosed technology are envisioned to shift the present paradigm from disparate acute or emergency medical treatments to remote and continuous monitoring and management for long-term diagnostic and predictive care. For example, acute management of a treatment site in the heart is presently left undetectable after treatment (e.g., implantation of stent, implant (such as a mitral annuloplasty, a mitral valve, a percutaneous mitral valve), pacemaker, etc.), unless a severe functional issue is “felt” by the patient that causes the patient to revisit the health provider, which in many cases is in an emergency situation. An in vivo fluid flow sensor device of the disclosed in vivo fluid flow sensor platform can be implanted directly on the periphery of the heart (and in a manner agnostic to any existing biomedical device implanted in the heart) to detect blood flow in the heart indicative of potential disparities, including, for example, cardiac output, stroke volume, total peripheral resistance, and/or development of restenosis.

For example, in some implementations, the disclosed in vivo fluid flow sensor device can measure fluid flow through the atrium chamber to the ventricle chamber of the heart, e.g., determine blood flow at the mitral valve for the left heart side or the tricuspid valve on the right side, and thereby determine fluid flow for a valve capacity known as “valve flow capacity” with measurement of the “Q”, i.e., flow rate (volume of blood/time) or the amount of pressure difference between the ends of the flow path divided by the resistance), which can be used for characterizing regurgitation of natural fluid blood flow, potential leaking by the valve, and back disruption flow. The disclosed in vivo fluid flow sensor devices, when placed proximate a heart valve, are able to detect where the valve dysfunction (e.g., leaking) is occurring, e.g., once placed in position and properly calibrated. Implementations of the disclosed in vivo fluid flow sensor platform (i.e., devices, systems, and/or techniques) can also be able to detect the diameter of the heart chamber or predictive area and/or predictive volume, as well as changes to a baseline measurement over time, which can indicate potential detrimental effects of heart function/performance with respect to that baseline and/or degradation of post treatment (e.g., that could lead to heart disease, such as Congestive Heart Failure (CHF), which is a chronic condition where the heart cannot pump blood as well as it should often due to weak or stiff tissue of the heart). For example, blood flow and regurgitation detection across the heart valve(s) can be used for correlation to heart function/performance and the effect of heart failure, as defined by CHF.

Also, for example, the data obtained by embodiments of the disclosed fluid flow sensor technology can be used in compilation with information to enable or optimize management of pharmaceutical prescription medication (e.g., dosage) for optimum patient care. For example, the data obtained by embodiments of the disclosed fluid flow sensor technology can be used for assessing valvular function and function degradation, as well as can be used in valvular treatment or vascular treatment (e.g., in compilation with EKG data). As an illustrative example, the disclosed in vivo fluid flow sensor platform can position an in vivo fluid flow sensor device on a vena cava and apply ultrasound signals to obtain information (e.g., dimensions of the vessel (thickness) and velocity of blood flow through the vena cava) that can be used to determine pulmonary wedge pressure, and thereby monitor the efficacy of certain medication doses in treating a patient with various forms of heart disease. Moreover, for patients with heart disease taking a cocktail of medications for managing their heart condition (e.g., diuretics for fluid volume, ace inhibitors for peripheral resistance, and beta blockers for contractility), blood pressure and flow through the vena cava can indicate whether the patient is properly complying with the medication regimen or whether the regimen is not optimal or effective, such as due to the body developing resistance to the drugs over time.

For example, the disclosed in vivo fluid flow sensor platform offers the capability of obtaining acoustic signal measurements of fluid flow directly through an anatomic structure (and without other anatomic structures in the acoustic signal pathway) and providing the obtained acoustic signal measurements to a remote device (e.g., such as a remote in vivo device in communication with the in vivo fluid flow sensor device and/or a remote device external to the body in communication with the in vivo fluid flow sensor device and/or the remote in vivo device), while also able to perform such functions with low power requirements and in a relatively small physical footprint (i.e., device volume) for long durations of time (e.g., years to over a decade).

While disclosed embodiments of an in vivo fluid flow sensor are described herein primarily based on monitoring of the blood flow across a heart structure (such as a heart valve) within a patient's body to facilitate understanding of the underlying concepts of the present technology, it is understood that the disclosed embodiments in accordance with the present technology can also include monitoring the dynamic flow of other biological fluids and other systems, including but not limited to fluid flow in the gastrointestinal system, renal system, or other.

In some embodiments, for example, a sensor device for in vivo monitoring of fluid flow in an anatomic structure while coupled to the anatomic structure, such as an atrium, ventricle, valve therebetween, or major blood vessel of the heart, includes a linkage assembly comprising a first arm configured to attach to a first portion of the anatomic structure and a second arm configured to attach to a second portion of the anatomic structure opposite to the first portion; a connection apparatus coupled to each of the first arm and the second arm; an ultrasound sensor assembly comprising a plurality of acoustic transducer elements coupled to the linkage assembly, the plurality of acoustic transducer elements including a first acoustic transducer element that is configured to transmit an acoustic signal to propagate through the anatomic structure and a second acoustic transducer element and a third acoustic transducer element that are configured to receive acoustic signals that have propagated through the anatomic structure and are indicative of a fluid flow parameter of a biological fluid in the anatomic structure; and an electronics unit housed in the connection apparatus and in electrical communication with the plurality of acoustic transducer elements of the ultrasound sensor assembly, the electronics unit configured to process electrical signals associated with the received acoustic signals as data and wirelessly transmit the data to an external processor.

In some embodiments, for example, a sensor device for in vivo monitoring of fluid flow in an anatomic structure while coupled to the anatomic structure, such as monitoring blood flow in an atrium, ventricle, valve therebetween, or major blood vessel of the heart, includes a first ultrasound assembly comprising a first set of one or more acoustic transducer elements; a second ultrasound assembly comprising a second set of one or more acoustic transducer elements; a first linkage coupled to the first ultrasound assembly; a second linkage coupled to the second ultrasound assembly; an electronics unit in electrical communication with the first ultrasound assembly and the second ultrasound assembly, the electronics unit configured to process the electrical signals associated with the returned acoustic signals as data and wirelessly transmit the data to an external processor; and a spring connection apparatus that couples to each of the first and second linkages and is operable to position the first ultrasound assembly at a first location on the anatomic structure and position the second ultrasound assembly at a second location on the anatomic structure to form a plane across the first and second locations of the anatomic structure to transmit and receive acoustic signals from the first set of one or more acoustic transducer elements and the second set of one or more acoustic transducer elements indicative of a fluid flow parameter of a biological fluid in the anatomic structure.

Some implementations of the disclosed devices, systems, and methods include in vivo monitoring of the state of a patient's cardiovascular and/or pulmonary disease for management of the patient's treatment and care of the disease, which includes heart failure, valvular disease, coronary disease, thoracic aortic aneurysm, chronic obstructive pulmonary disease (COPD), sleep apnea, asthma, and/or other acute or chronic disease or conditions. In particular, the disclosed implantable medical devices, systems, and methods of the present technology can be implemented to assist the patient's health care provider (HCP) in the medication management for the patient's disease or condition.

As an example, some embodiments of an exemplary IMD can be implemented to acoustically monitor cardiovascular function using a high fidelity implantable acoustic sensor of the IMD for continuously detecting abnormal blood flow associated with aortic stenosis (AS) and mitral valve regurgitation (MVR) for valvular disease drug management. Also, as an example, some embodiments of an exemplary IMD can be implemented to acoustically monitor both cardiovascular and pulmonary function using the IMD's high fidelity acoustic sensor for continuously detecting abnormal blood flow associated with AS, MVR, and other heart valve acoustics in conjunction with or exclusive from pulmonary acoustics associated with breathing, including but not limited to edema in the lungs, asthma, sleep apnea, COPD, or other pulmonary conditions for cardiovascular and pulmonary disease drug management.

For example, the disclosed implantable medical devices, systems, and methods that include the in vivo fluid flow sensor and the in vivo acoustic sensor, of the present technology, can contemporaneously provide clinical data the patient's HCP that pertains to contractility, resistance, and/or volume of blood pumped by the patient's heart such that the HCP can make immediate decisions to impact medications for managing the cardiovascular disease or condition. For example, an IMD comprising the in vivo fluid flow sensor can continually measure cardiac output in compilation with continual measurement of mechanical waves (acoustics) associated with stenosis or regurgitation by the in vivo fluid flow sensor-which can be processed in real time based on the cardiac cycle characterized by an (auxiliary) in vivo ECG sensor-which together can provide a complete, on-going assessment of the patient's cardiovascular health to characterize heart function/dysfunction and the continual assessment of the effectiveness of the patient's treatment regime, including compliance thereto.

Presently, many forms of heart disease are treated with specific doses of one or more of (i) angiotensin-converting enzyme (ACE) inhibitors, i.e., medicines that relax the veins and arteries to lower blood pressure by preventing an enzyme required in the production of angiotensin 2; (ii) beta-adrenergic blocking agents (Beta blockers), by blocking the effects of the hormone epinephrine to cause the heart to beat more slowly and with less force, which can also result in widening veins and arteries to improve blood flow; and (iii) diuretics, i.e., medicines that can lower the amount of fluid flowing through the veins and arteries, thereby lowering blood pressure, and that can reduce fluid buildup in the body, e.g., promoting kidney function for salt and water removal through urine. These drugs are given to patients with heart disease, particularly at stages 3 or 4, in order to prevent the patient from decompensating, i.e., undergoing decompensated heart failure (DHF), which is where a structural or functional change in the patient's heart leads to heart's inability to eject and/or accommodate blood within physiological pressure levels, thus causing a life-threatening heart function limitation and requiring immediate therapeutic intervention (e.g., emergency room). Some DHF symptoms include the patient's inability to breathe, low pulse oximetry, high heart rate, or other, which can precede an organ system failure.

Among the greatest challenges for patients and their HCPs in managing their cardiovascular and/or pulmonary disease conditions is patient compliance. Patients must comply with two facets of their own health care: (1) regularly (e.g., multiple times daily) take their prescribed medications precisely when and how they are prescribed, and (2) regularly (e.g., one or more times daily) measure and record multiple physiological measurements, including blood pressure (BP), body weight (BW), and blood oxygen level (PulseOx). Current heart monitoring systems are a compilation of external sensors, such as a blood pressure cuff (digital), body weight scale (digital), and pulse oximeter (digital), which require discrete and separate measurements of the patient's BP, BW, and PulseOx, respectively. Yet, when patients comply, the system can gauge risk levels for whether the patient is successfully managing his/her heart disease or trending toward DHF. For instance, if a patient is developing a lot of fluid in his/her lungs and the patient's heart rate is increasing, then the patient's BW increases and is measurable by the body weight scale and the patient's heart rate increase and is measurable by the blood pressure cuff-which can collectively indicate the patient may be decompensating and in need of immediate medical attention (e.g., trip to emergency room where an HCP can adjust the concentrations of the ACE inhibitors, Beta blockers, and diuretics for avoiding DHF).

While studies have shown patients generally can be relied upon to regularly take their medications as prescribed, most patients struggle to keep with the strict protocols to measure and record their BP, BW, and PulseOx. The conventional approach for treating cardiovascular and pulmonary disease is severely flawed because of the reliance on patient compliance. The disclosed implantable medical devices, systems, and methods of the present technology can be implemented to continuously, passively, and autonomously (i.e., no patient interaction) monitor, from within the patient's body, a multitude of physiological markers associated with cardiovascular and/or pulmonary health and disease, thereby taking patient compliance out of the equation for the physiological monitoring component of the patient's treatment and care.

The disclosed implantable medical devices, systems, and methods of the present technology can continuously, autonomously, and passively monitor heart rate, fluid flow and accumulation in tissues or organs, patient movement and activity, electrophysiological signals like an electrocardiogram (ECG or EKG), and other physiological measurements based on the IMD's in vivo acoustic and/or fluid flow sensor(s), which can be configured in conjunction with one or more auxiliary sensors, a motion sensor or inertial measurement unit, an electrophysiological sensor, or other, as disclosed herein. For example, in some implementations of the IMD using the acoustic sensor and an ECG sensor as a temporal qualifier, i.e., a timing marker for events during the cardiac cycle, the IMD can interrogate for particular sounds to distinguish between healthy and unhealthy markers of cardiac function.

The in vivo acoustic, fluid flow, and auxiliary sensors of an IMD of the present technology are not blood-contacting, i.e., the sensors are engineered to detect physiological phenomena, particularly including those associated with blood flow, without being positioned inside blood vessels. For example, once the IMD, having the in vivo fluid flow sensor, the in vivo acoustic sensor and certain auxiliary sensors (e.g., such as the ECG and IMU), is implanted proximate the patient's heart, the IMD can monitor twelve disease states for each of the four valves of the heart, e.g., (1) stenosis (narrowing of the valve in a large blood vessel branching off or into the heart, which typically is caused due to calcification); (2) regurgitation (backwards flow of blood through due to a valve defect); and (3) myxomatous, a combination of stenosis and regurgitation (degeneration of the cardiac valves). The continuous, autonomous, and passive in vivo monitoring capability by the disclosed embodiments of the IMDs of the present technology is important for the advancement of cardiovascular healthcare because none of these disease states are easy to observe presently due to the fact that it is the change over time in these disease states that allows for their diagnosis, and conventional diagnostic systems, devices, and techniques are incapable of or severely limited to accurately, reliably, and conveniently track the physiological markers over such time durations to effectively and optimally enable HCPs to make proper diagnoses. In addition, the IMD of the present technology can utilize its IMU sensor(s) to controllably collect data at both a resting state and a state of activity to simulate a stress test, thereby increasing the IMD's capability to assess patient disease conditions. In such implementations, the disclosed embodiments of the IMD of the present technology are capable of inconspicuously characterizing cardiovascular and/or pulmonary function in multiple states and situations of the patient's condition-without reliance on patient compliance.

In some implementations of the IMD of the present technology, for example, the IMD including the in vivo acoustic sensor can operate like an internal stethoscope for the HCP that is continuously measuring patient respiration, which not only can be used to monitor pulmonary conditions such as asthma or COPD, but can also pick up mitral valve regurgitation and heart failure because the breathing acoustics are changed when there is fluid in the lungs, and MVR and heart failure can cause such fluid accumulation. Thus, the disclosed embodiments of the in vivo acoustic sensor of the present technology can supplement or replace external digital stethoscopes, which suffer from poor fidelity and reliability (poor compliance) due to over 90% signal loss from transmission of the acoustic signals from the tissues of the body through air to be transduced by the external microphone of the digital stethoscope. In some embodiments, the in vivo acoustic sensor of the present technology can sense an acoustic signal having a frequency of from 1 Hz, or 2 Hz, or 10 Hz, up to about 20,000 Hz, or up to about 18,000 Hz, or up to about 16,000 Hz, or up to about 14,000 Hz, or up to about 12,000 Hz, or up to about 10,000 Hz. For example, various embodiments of an exemplary IMD that includes the in vivo acoustic sensor can be configured to detect acoustic signals in a frequency range of or within 0.1 Hz to 20 kHz, where specific ranges can be defined based on the particular embodiment of the acoustic sensor.

In some embodiments of an IMD that includes an in vivo acoustic sensor in accordance with the present technology, for example, the in vivo acoustic sensor includes a displacement-mediated acoustic sensor to measure a change in conformation caused by a mechanical wave on a transducer material. In some embodiments, for example, the in vivo acoustic sensor includes a microphone. In some embodiments, for example, the in vivo acoustic sensor can include an accelerometer. In some embodiments, for example, the in vivo acoustic sensor may include a diaphragm which comes into contact with the sound wave and vibrates or otherwise extends in response to the sound wave, where the extension is measured over time to provide a temporal measurement of sound. In some embodiments, for example, the in vivo acoustic sensor includes a strain gauge operable to detect strain and/or deformation of a material by measuring resistivity. In some embodiments, for example, the in vivo acoustic sensor includes a piezoelectric sensor operable to measure changes in pressure, acceleration, temperature, strain, or force by converting them to an electrical charge.

In some embodiments of an IMD that includes an in vivo acoustic sensor in accordance with the present technology, for example, the in vivo acoustic sensor includes a stress-mediated acoustic sensor operable to measure stress applied to a transducer material. In some embodiments, the acoustic sensor includes a stress-mediated microelectromechanical sensor (MEMS) device to transduce mechanical waves that apply force on the MEMS device into electrical signals. Some examples of a MEMS stress-mediated acoustic sensor can include a piezoelectric stress sensor, and in some embodiments, for example, the piezoelectric stress sensor includes a unimorph piezoelectric sensor device.

These and other embodiments of the disclosed devices, systems, and methods are discussed in greater detail by the examples below.

### EXAMPLE EMBODIMENTS

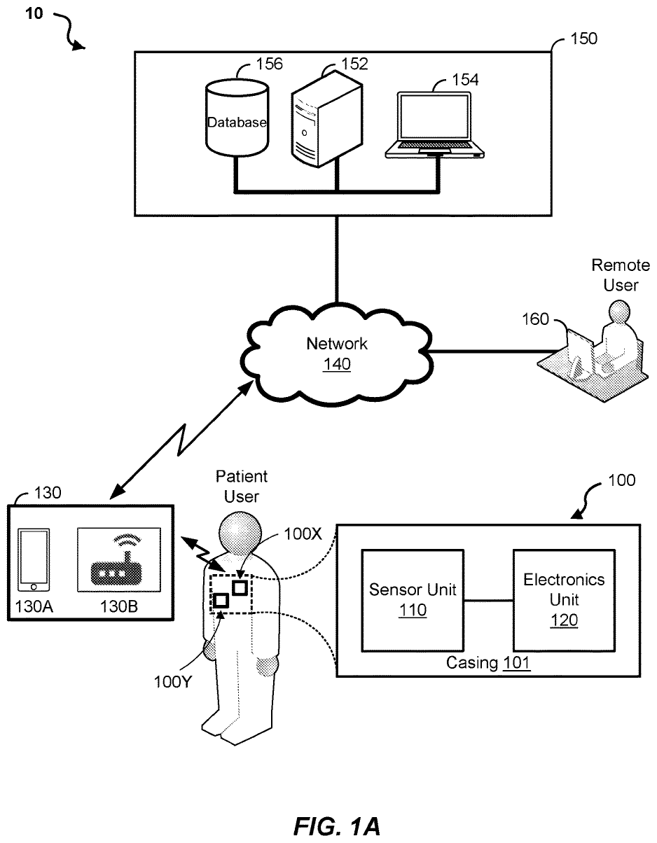

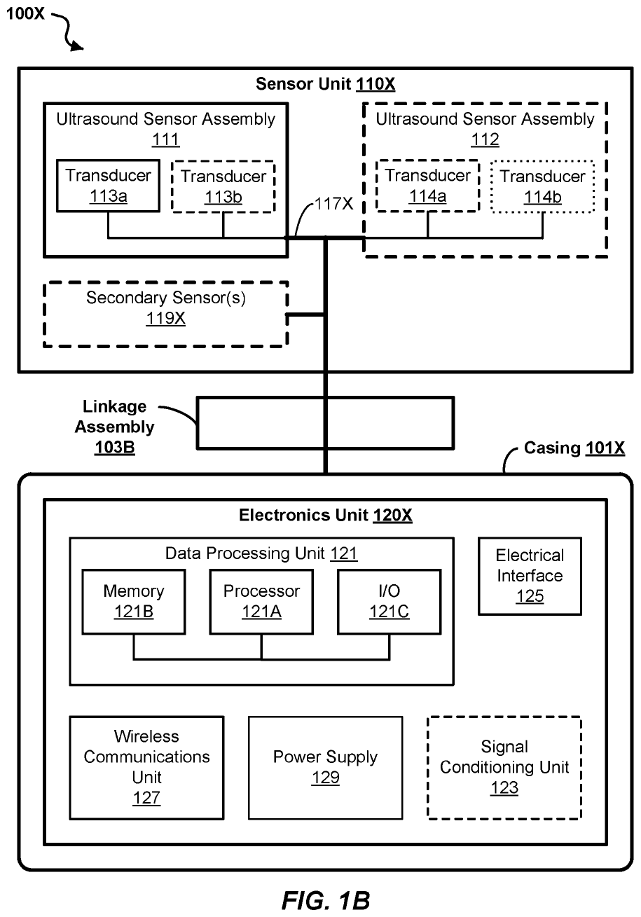

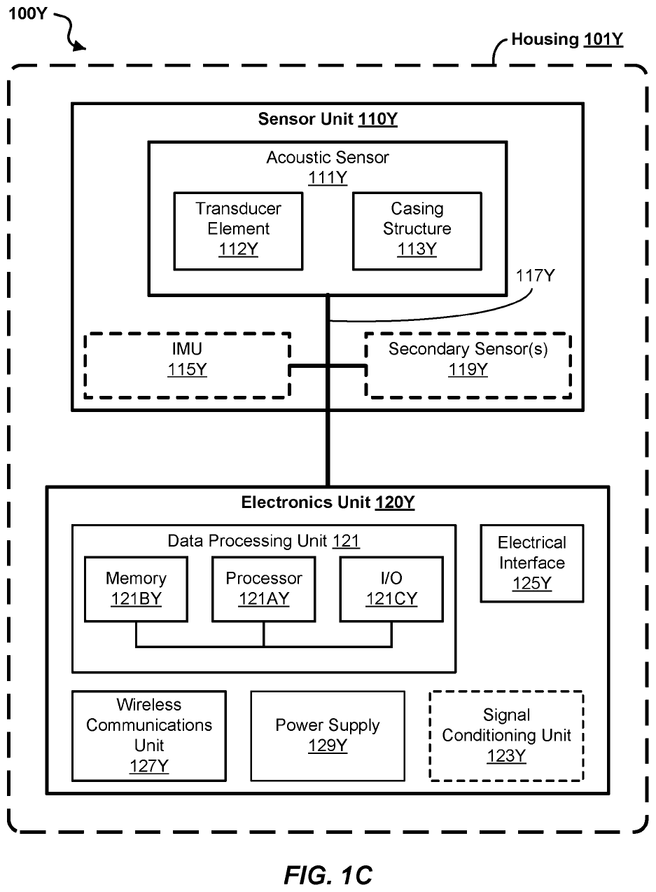

FIG. 1A shows a diagram illustrating an example embodiment of a system 10 for in vivo monitoring of fluid flow in a patient user, in accordance with the present technology. The system 10 includes implantable medical device (IMD) 100 that include one or more in vivo fluid flow sensor devices 100X and/or one or more in vivo acoustic sensor devices 100Y, which can be implanted within the patient user, and a data processing system 150 in communication with the one or more in vivo fluid flow sensor devices 100X and/or the one or more in vivo acoustic sensor devices 100Y. In some embodiments, the system 10 includes a receiver device 130 operable to (i) receive a wireless transmission carrying data indicative of detected signals acquired from the one or more in vivo fluid flow sensor devices 100X and/or the one or more in vivo acoustic sensor devices 100Y and (ii) transmit to and/or store the data, e.g., transmit the data to the data processing system 150. In some embodiments, the implantable medical device 100 and/or the receiver device 130 is/are in communication with the data processing system 150 via a network 140 of computers in communication with each other and accessible through the Internet (e.g., referred to as the cloud), where the data from the implantable medical device 100 and/or the receiver device 130 can be transferred to the data processing system 150. Similarly, information from the data processing system 150 can be transferred to the receiver device 130 and/or the implantable medical device 100. For example, the data processing system 150 can manage data compilation(s) into a directional or changing modality to provide a continuous, long-term representation (i.e., far more than a moment representation) informative of the patient's health and disease, and which can also provide a delta change representation (whether on a short-term or long-term temporal scale) to aide in the analysis of the patient's health and disease from a point in time (e.g., any point in time defined as an initial point (To)) to a point in future time (Tr), thereby functionally providing deterioration or improvement of the physical state from a point of implant and treatment of the one or more in vivo fluid flow sensor devices 100X and/or the one or more in vivo acoustic sensor devices 100Y of the implantable medical device 100.

The implantable medical device 100 includes a sensor unit 110 and an electronics unit 120, which one or both of the sensor unit 110 and the electronics unit 120 are housed in and/or coupled via a housing or casing 101. The sensor unit 110 and the electronics unit 120 of the implantable medical device 100 can be configured as one unit for each of the one or more in vivo fluid flow sensor devices 100X and/or the one or more in vivo acoustic sensor devices 100Y of the implantable medical device 100, or can be configured as multiple separate or partially-shared and partially-separate units for the one or more in vivo fluid flow sensor devices 100X and/or the one or more in vivo acoustic sensor devices 100Y of the implantable medical device 100. In some examples, the housing or casing 101 is configured to shield components of the electronics unit 120 from fluids or substances of the body when the implantable medical device 100 (e.g., in vivo fluid flow sensor device 100X and/or in vivo acoustic sensor device 100Y) is deployed inside the patient user.

In the example of the system 10 illustrated in FIG. 1A, the implantable medical device 100 includes a first implantable device: at least one in vivo fluid flow sensor device 100X deployed in a first portion of the patient user's body, e.g., the chest, head, torso, an appendage, or other area; and the implantable medical device 100 includes a second implantable device: at least one in vivo acoustic sensor device 100Y deployed in a second portion of the patient user's body. One or both of the in vivo fluid flow sensor device 100X and/or the in vivo acoustic sensor device 100Y, respectively, may be directly coupled to an organ or tissue within the patient's body at the first portion and/or second portion, respectively. In some implementations, for example, the first in vivo fluid flow sensor device 100Y may be coupled to an implant device deployed at or near the first portion in the patient user's body, such as an implant for the heart, lung, cranium, neck, intestines and digestive track, limb or extremity, etc. Similarly, for example, a second device of either or both of the in vivo fluid flow sensor device 100X and/or the in vivo acoustic sensor device 100Y may be deployed at or near a third portion in the patient user's body or at or near one of the first portion or the second portion where the in vivo fluid flow sensor device 100X and the in vivo acoustic sensor device 100Y are deployed, respectively.

In some embodiments of the implantable medical device 100, for example, the one or more in vivo fluid flow sensor devices 100X and/or the one or more in vivo acoustic sensor devices 100Y can be configured as part of a single device structure (e.g., shared housing); whereas in some embodiments, for example, the one or more in vivo fluid flow sensor devices 100X and/or the one or more in vivo acoustic sensor devices 100Y can be configured as part of separate device structures. In such embodiments with separate device structures, the one or more in vivo fluid flow sensor devices 100X and/or the one or more in vivo acoustic sensor devices 100Y can be electrically connected to each other; and in some embodiments, the separated in vivo fluid flow sensor device(s) 100X and the in vivo acoustic sensor device(s) 100Y can be configured to share electronic components, e.g., including but not limited to a power supply, wireless communication unit, data processing unit, or other electronic resources and functionalities.

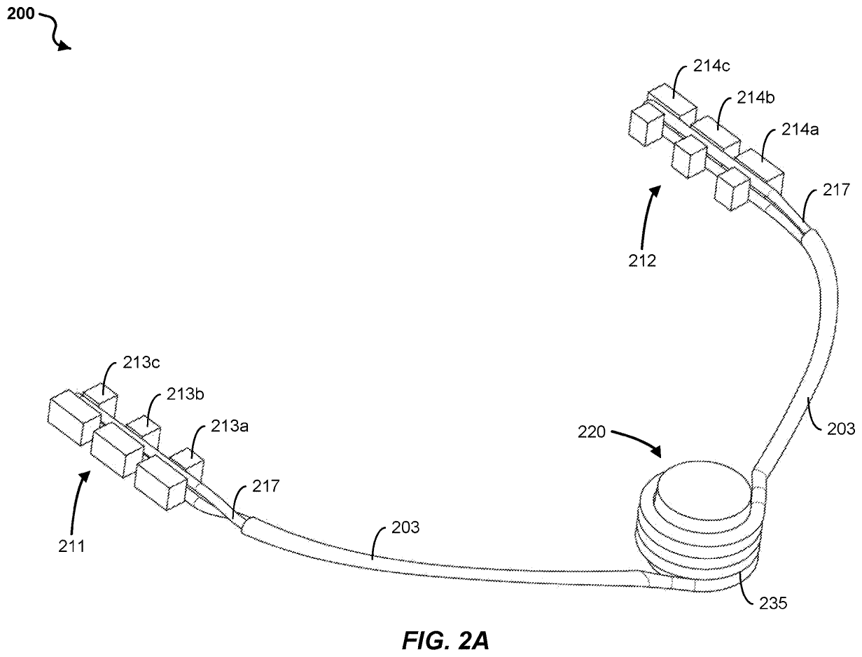

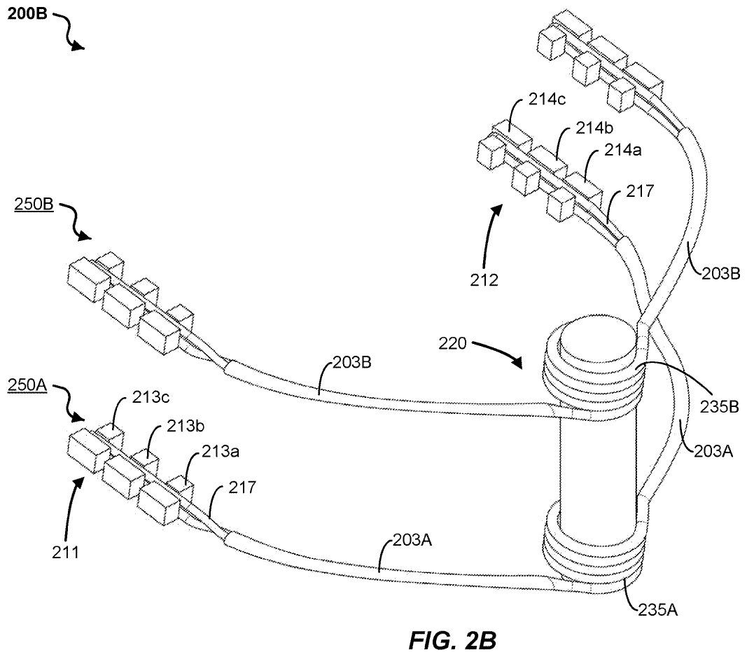

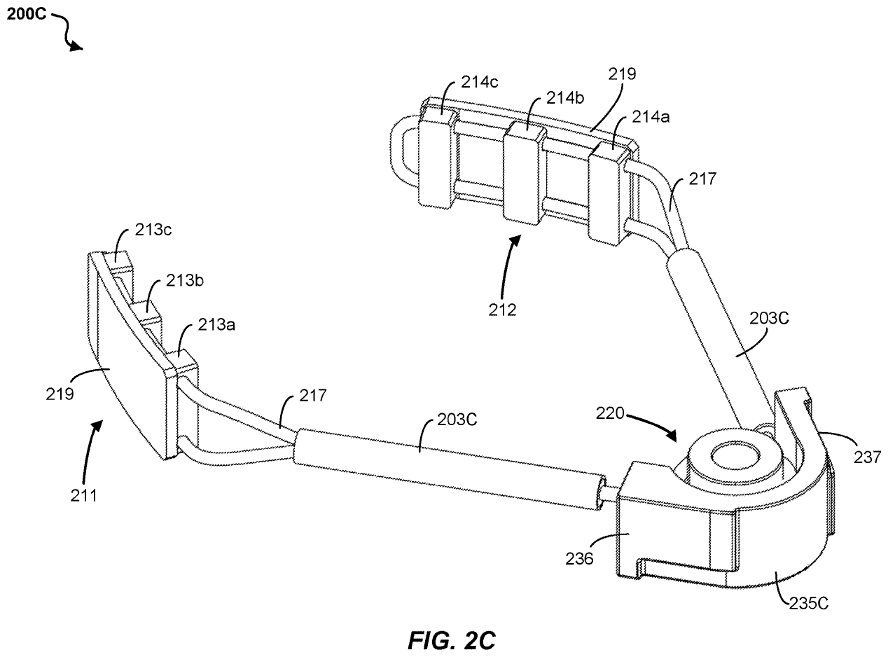

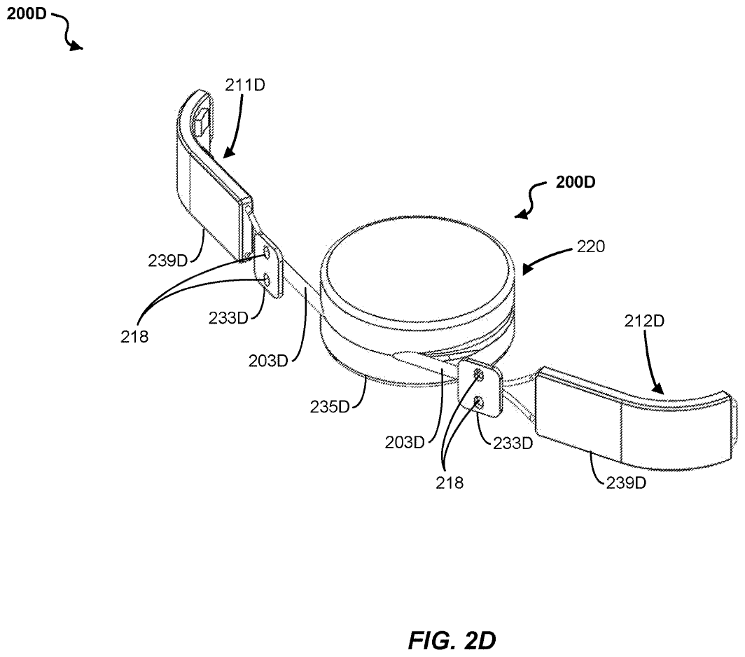

In some example embodiments of the in vivo fluid flow sensor device 100X, such as those discussed later in connection with FIG. 1B, the sensor unit 110 includes at least one of a first ultrasound sensor assembly and a second ultrasound sensor assembly each including one or more acoustic transducer elements, which are configured to be positioned across and in contact with an anatomic structure, such as the heart, and electrically connected to the electronics unit 120 (e.g., housed in the casing 101), such that one or both of the first and second ultrasound sensor assemblies transmit and receive acoustic signals across a region of interest of the anatomic structure and the electronics unit 120 processes and/or relays the data associated with the monitored acoustic signals to the receiver device 130. The one or more acoustic transducer elements of the first and second ultrasound sensor assemblies of the sensor unit 110 are electrically coupled to the electronics unit 120, e.g., via electrical interconnections such as wires, to provide electrical signals to stimulate transmission of the acoustic transmit signals across the region of interest and to receive electrical signals transduced from received acoustic signals propagating across the region of interest of the anatomic structure. Further details of the sensor unit 110 and the electronics unit 120 for some embodiments of the in vivo fluid flow sensor device 100X are discussed in FIG. 1B and other figures including FIGS. 2A-10B.

Referring to FIG. 1A, in some implementations, the implantable medical device 100 (e.g., in vivo fluid flow sensor device 100X and/or in vivo fluid flow sensor device 100Y) wirelessly communicate the acquired data obtained directly to the receiver device 130. For example, the implantable medical device 100 can transfer the data to the receiver device 130 using a low power wireless communication protocol, e.g., such as Bluetooth Low Energy (BLE), Near Field Communication (NFC), low frequency radio frequency (RF) signal in a range of 3 kHz to 1.3 MHz, or other. Example embodiments of the receiver device 130 include a computing device 130A or a dedicated base station 130B. For example, the computing device 130A can include, but is not limited to, a smartphone, tablet, a home device (e.g., Alexa, Nest, Echo, Google Home, Smart TVs, etc.), a wearable computing device (e.g., smartwatch, smart-glasses or headgear, etc.), a laptop or desktop computer, or other. The dedicated base station 130B can include data storage and/or data communication units that facilitate the communication of data from the implantable medical device 100 to the data processing system 150 through a Wi-Fi access or cellular link to the network 140. In some implementations, for example, the receiver device 130 can be embodied on multiple receiver devices, such both the computing device 130A (e.g., smartphone, tablet, etc.) and the dedicated base station 130B, as illustrated in the example of FIG. 1A. In some implementations, for example, the receiver device 130 can (i) process, at least partially, the received data for display on a display screen of the receiver device 130 and/or for transfer of the received data to an external computer or computing system, such as the data processing system 150. In some embodiments, for example, the system 10 optionally includes a software application (“app”) that is resident on the receiver device 130 to control various data processing, storage, and communication functionalities for management of the received data.

In the example of the system 10 illustrated in FIG. 1A, the data processing system 150 can include one or more server computer devices 152, one or more client computer devices 154, and/or one or more databases 156, in data communication with each other. In implementations, for example, the computer device(s) 152, 154 and the database(s) 156 are in communication with each other and/or in communication with the other devices of the system 10 via the network 140. In some implementations, for example, the data processing system 150 can remotely monitor data associated with the patient user obtained by the implantable medical device 100 and/or remotely operate aspects of the system 10, e.g., such as modify sensing parameters or protocols of the one or more in vivo fluid flow sensor devices 100X and/or the one or more in vivo acoustic sensor devices 100Y, data display or processing features of the app on the receiver device 130, or other.

In some embodiments, for example, the system 10 optionally includes a remote computing device 160 operated by a remote user to remotely monitor data associated with the patient user obtained by the implantable medical device 100 that is transferred to the data processing system 150. For example, the remote computer 160 can include a personal computer such as a desktop or laptop computer, a mobile computing device such as a smartphone, tablet, smartwatch, etc., or other computing device. In some implementations, for example, the remote computing device 160 is configured to only receive data that is curated (e.g., selected, pre-processed, and/or formatted) by the data processing system 150. In some implementations, for example, the remote computing device 160 is configured to remotely operate one or more aspects (e.g., functionalities) of the system 10. For example, the remote computing device 160 can implement a remote user software application (remote user app) that is configured to provide the remote user with such display, storage, and/or management features. The remote user, for example, can include a health care provider (HCP), such as a physician, nurse, family member of the patient user, or other caregiver, or a medical insurance payer, or other type of stakeholder entity or individual with respect to the patient user's health.

FIG. 1B shows a block diagram illustrating an example embodiment of the in vivo fluid flow sensor 100X shown in FIG. 1A. In the example of FIG. 1B, the in vivo fluid flow sensor 100X includes an example embodiment of a sensor unit 110 (shown as sensor unit 110X) and an example embodiment of electronics unit 120 (shown as electronics unit 120X). The sensor unit 110X of the in vivo fluid flow sensor 100X includes at least one ultrasound assembly in electrical communication with the electronics unit 120X via electrical interconnection(s) 117X, which is depicted in FIG. 1B as having a first ultrasound sensor assembly 111 and an optional second ultrasound sensor assembly 112, each in electrical communication with the electronics unit 120X via the electrical interconnection(s) 117x. The first ultrasound sensor assembly 111 includes one or more acoustic transducer element(s) 113 (also referred to herein as “transducer 113” or “one or more transducers 113”); and the optional second ultrasound sensor assembly 112 includes one or more acoustic transducer element(s) 114 (also referred to herein as “transducer 114” or “one or more transducers 114”). The one or more transducers 113 of the first ultrasound sensor assembly 111 includes a transducer element 113a and may optionally include an additional transducer element or elements, represented in FIG. 1B as transducer element(s) 113b. Similarly, the one or more transducers 114 of the optional second ultrasound sensor assembly 112 includes a transducer element 114a and may optionally include an additional transducer element or elements, represented in FIG. 1B as transducer element(s) 114b. For example, embodiments with multiple acoustic transducer elements can be configured as an array of transducers of the respective ultrasound sensor assembly. The electrical interconnection(s) 117X are configured to couple each of the one or more acoustic transducer element(s) 113 of the first ultrasound sensor assembly 111 and the one or more acoustic transducer element(s) 114 of the optional second ultrasound sensor assembly 112 to an electrical interface of the electronics unit 120x. In various implementations of example embodiments of the in vivo fluid flow sensor 100X, the first ultrasound sensor assembly 111 and the optional second ultrasound sensor assembly 112 may transmit and receive acoustic signals at one or more frequencies within a frequency range of 2 MHz to 20 MHz. In various embodiments, for example, the first ultrasound sensor assembly 111 and the optional second ultrasound sensor assembly 112 each may include a frame, casing or housing structure (not shown) to structurally support the one or more acoustic transducer element(s) 113 and the one or more acoustic transducer element(s) 114, respectively, and position them in a fixed location with respect to each other.

In some embodiments, for example, the transducer element or elements of the one or more acoustic transducer element(s) 113 and the one or more acoustic transducer element(s) 114 include a piezoelectric transducer operable to transmit acoustic signals based on an electrical input signal and receive acoustic signals to produce an electrical output signal. For example, the piezoelectric transducer can include a solid piezoelectric ultrasound transducer or a piezoelectric micromachined ultrasonic transducer (PMUT), e.g., a MEMS-based piezoelectric ultrasonic transducers for acoustic imaging of the environment. In some embodiments, for example, the transducer element or elements of the one or more acoustic transducer element(s) 113 and the one or more acoustic transducer element(s) 114 include a ferroelectric hafnium oxide transducer.

The in vivo fluid flow sensor device 100X includes a linkage assembly 103B that couples the first ultrasound sensor assembly 111 to the electronics unit 120X (and, in embodiments including the second ultrasound sensor assembly 112, couples the second ultrasound sensor assembly 112 to the electronics unit 120X). In some embodiments, the linkage assembly 103B can include a pair of linkages that are configured to (1) secure (e.g., attach and anchor) the in vivo fluid flow sensor device 100X to the target in vivo organ or tissue and (2) carry the respective electrical interconnection(s) 117X (connected to the transducer(s) 113 and/or the transducer(s) 114) from the first ultrasound sensor assembly 111 and/or the optional second ultrasound sensor assembly 112 to the electronics unit 120x. In some embodiments, for example, the pair of linkages of the linkage assembly 103B include a spring connection apparatus, discussed later in connection with FIG. 2A. Whereas, in some embodiments, for example, the pair of linkages of the linkage assembly 103B include a flexible band, with or without a spring, as discussed later in this patent document.

In some embodiments of the in vivo fluid flow sensor device 100X, for example, the sensor unit 110X may optionally include one or more secondary sensor(s) 119X. For instance, in some implementations, the secondary sensor(s) 119X may include an analyte sensor to measure a parameter (e.g., concentration) of an analyte in the region proximate the anatomic structure where the in vivo fluid flow sensor device 100X is deployed. In some examples, the optional secondary analyte sensor can include, but is not limited to, a glucose sensor. Additionally or alternatively to an optional secondary analyte sensor, the secondary sensor(s) 119X may optionally include a pH sensor to measure the pH level in the region proximate the anatomic structure. Additionally or alternatively to a secondary analyte sensor and/or secondary pH sensor, the secondary sensor(s) 119X may optionally include a temperature sensor to measure the temperature in the region proximate the anatomic structure. In such cases, for example, the optional one or more secondary sensor(s) 119X comprising the analyte sensor, the pH sensor, and/or the temperature sensor can be used to obtain data indicative of clinically-relevant conditions about the anatomic structure and/or conditions of the in vivo environment where the in vivo fluid flow sensor device 100X is deployed, such as a potential infection or inflammatory response to the implantation of the in vivo fluid flow sensor device 100X. In some embodiments, for example, the optional one or more secondary sensor(s) 119X can be attached to a structure of an example embodiment of the casing 101 (e.g., casing 101X of the in vivo fluid flow sensor device 100X), e.g., via weld, chemical adhesion, clip, clamp, or other attachment means; and in some embodiments, for example, the optional one or more secondary sensor(s) 119X can be attached to one or both of the first ultrasound sensor assembly 111 and/or the (optional) second ultrasound sensor assembly 112, e.g., via attachment to the frame or casing structure.

In some embodiments, for example, the optional one or more secondary sensor(s) 119X of the in vivo fluid flow sensor device 100X can include an inertial measurement unit (IMU) that is configured to monitor motion of the in vivo fluid flow sensor device 100X in multiple degrees of freedom. In some embodiments, for example, the optional one or more secondary sensor(s) 119X of the in vivo fluid flow sensor device 100X can include an accelerometer in communication with a data processing unit of the electronics unit 120X. In some embodiments, for example, the optional one or more secondary sensor(s) 119X of the in vivo fluid flow sensor device 100X can include a rate sensor in communication with a data processing unit of the electronics unit 120x. In some embodiments, for example, the optional one or more secondary sensor(s) 119X of the in vivo fluid flow sensor device 100X can include a magnetometer in communication with a data processing unit of the electronics unit 120X. In such implementations, for example, the in vivo fluid flow sensor device 100X is operable to measure heart rate in compilation with fluid flow through the heart (e.g., across the mitral valve or tricuspid valve) when the in vivo fluid flow sensor device 100X is deployed on the exterior of the heart, e.g., in the pericardium.

Also shown in the example of FIG. 1B, the electronics unit 120X of the in vivo fluid flow sensor device 100X includes a data processing unit 121, an optional signal conditioning unit 123, a power supply 129, a wireless communications unit 127, and an electrical interface 125, which can include electrically conductive contact sites (e.g., pads, pins, or other contact configuration) that electrically interfaces with the electrical interconnections 117X of the sensor unit 110X. The electronics unit 120X is configured to receive and at least partially process electrical signals acquired from the one or more acoustic transducer(s) 113 of the first ultrasound sensor assembly 111 and the one or more acoustic transducer(s) 114 of the second ultrasound sensor assembly 112 of the sensor unit 110X. For example, in some embodiments, the electrical signals are received at the corresponding contact sites of the electrical interface 125 and provided to the data processing unit 121 (or, optionally, first to the signal conditioning unit 123 to improve the quality of the acquired electrical signals from the sensor unit 110X prior to providing to the data processing unit 121). In such implementations, the output of the data processing unit 121 can include raw or processed data associated with the detected data from the sensor unit 110X, to be wirelessly transmitted to an external device by the wireless communications unit 127. In example embodiments of the electronics unit 120X, the power supply 129 can include a battery (e.g., primary or rechargeable), fuel cell or other power source to supply power to the components of the electronics unit 120X and/or the sensor unit 110X. In some implementations, for example, the power supply 129 includes an ultra-low power system (e.g., operating in the micro amp range).

In some optional embodiments, for example, the signal conditioning unit 123 can include a circuit including one or more filters and/or one or more amplifiers to augment the raw electrical signals detected by the ultrasound sensor assemblies 111, 112 of the sensor unit 110X to increase a signal-to-noise ratio (SNR) of the electrical signals, thereby producing data containing the signal-processed electrical signals. In some optional embodiments, the signal conditioning unit 123 can include drive circuitry to produce operating electrical signals that generate electrical potentials and/or currents at the optional analyte sensor electrode contingent(s) and/or temperature sensor contingent of the sensor unit 110X for operating an electrochemical sensing technique and/or electrophysiological or kinetic sensing technique to be performed at electrode(s) in implementations of the optional secondary sensor(s) 119X of the sensor unit 110X.

In some embodiments, for example, the wireless communications unit 127 includes a wireless transmitter, receiver, and/or transceiver device including an antenna, which is capable of communicating with an external device to communicate raw, partially-processed, or fully-processed data from the signal conditioning unit 123 (and/or the data processing unit 121, discussed below). For example, the wireless communications unit 127 can be configured to manage the communication protocol for transmission or reception via the antenna. Examples of antenna can include, but are not limited to, a whip antenna, a loop antenna, or a conformal antenna. An example transceiver unit can include a BLE chipset to communicate with a BLE-enabled device, e.g., a smartphone, tablet, or other external computing device, such as of the receiver device 130. Additionally or alternatively, in some embodiments, for example, the wireless communications unit 127 is configured as a scaffolding around the electronics unit 120X, e.g., such as coupled to or integrated with the casing 101X, that is structured to provide wireless communication means for the in vivo fluid flow sensor device 100X.

In some embodiments, the electronics unit 120X includes a data processing unit 121 to at least partially process the conditioned electrical signals to (i) produce data, e.g., in an analog or a digital form, and/or (ii) control functionality of the electronics unit 120X and/or the sensor unit 110X. For example, the data processing unit 121 can be configured to manage data acquisition on data channels associated with the one or more acoustic transducers 113 and the one or more acoustic transducers 114 of the sensor unit 110X.

In some embodiments of the data processing unit 121, for example, the data processing unit 121 can include a processor 121A to process data and a memory 121B in communication with the processor 121A to store and/or buffer data. In various embodiments, for example, the processor 121A can include one or multiple processors, and the memory 121B can include one or multiple memory units. For example, the processor 121A can include a central processing unit (CPU), a microcontroller unit (MCU), a graphics processing unit (GPU), or other type of processor. For example, the memory 121B can include and store processor-executable code, which when executed by the processor, configures the data processing unit 121 to perform various operations, e.g., such as receiving information, commands, and/or data, processing information and data, and transmitting or providing information/data to another device. To support various functions of the data processing unit 121, the memory 121B can store information and data, such as instructions, software, values, images, and other data processed or referenced by the processor 121A. For example, various types of random access memory (RAM) devices, read only memory (ROM) devices, flash memory devices, and other suitable storage media can be used to implement storage functions of the memory 121B. In some embodiments, the data processing unit 121 includes an input/output (I/O) unit 121C to interface the processor 121A and/or memory 121B to other modules, units or devices, e.g., associated with an external device, such as the receiver device 130, the data processing system 150, the remote computing device 160, and/or other external devices. In some embodiments, the processor 121A, the memory 121B, and/or the I/O unit 121C is in communication with the wireless communications unit 127, e.g., such as a transmitter (Tx) or a transmitter/receiver (Tx/Rx) unit. For example, in such embodiments, the I/O unit 121C can interface the processor 121A and memory 121B with the wireless communications unit 127, e.g., to utilize various types of wireless interfaces compatible with typical data communication standards, which can be used in communications of the data processing unit 121 with other devices. The data communication standards include, but are not limited to, Bluetooth, Bluetooth Low Energy (BLE), Zigbee, Medical Implant Communication Service (MICS), industrial, scientific, and medical (ISM) band, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE/5G/6G cellular communication methods, and parallel interfaces. In some implementations, the data processing unit 121 can interface with other devices using a wired connection via the I/O unit 121C, e.g., such as prior to implantation within the patient user. The data processing unit 121 can also interface with other external interfaces, sources of data storage, and/or visual or audio display devices, etc. to retrieve and transfer data and information that can be processed by the processor 121A, stored in the memory 121B, or exhibited on an output unit of the receiver device 130 (e.g., smartphone, tablet, etc.) or other external device to the in vivo fluid flow sensor device 100X.

In implementations of the in vivo fluid flow sensor device 100X, the first ultrasound sensor assembly 111 (and the optional second ultrasound sensor assembly 112) is/are controlled by the data processing unit 121 to transmit and receive acoustic signals. For example, in some implementations, the in vivo fluid flow sensor device 100X is configured to measure the Doppler shift of acoustic probe signals across the anatomic structure. For instance, the traveling time of the acoustic signals is indicative of an estimated distance of travel, and the frequency shift of a received acoustic signal is proportional to the velocity of the fluid flowing along the acoustic path. The data processing unit 121 is configured to provide a set of electrical control signals to the one or more transducers 113 and/or the one or more transducers 114 to stimulate transmission of acoustic probe signals across the region of interest, such as the mitral valve of the heart, such that the opposing set of transducers among the one or more transducers 113 and/or the one or more transducers 114 receives the fluid flow shifted acoustic probe signals; and based on the time of flight of the acoustic probe signals and known fixed positions of the one or more transducers 113 and the one or more transducers 114, the data processing unit 121 process the electrical signals transduced from the one or more transducers 113 and/or the one or more transducers 114 associated with the received acoustic probe signals to determine the fluid flow, e.g., forward flow or possible backward flow of blood across the mitral valve of the heart.

FIG. 1C shows a block diagram illustrating an example embodiment of the in vivo acoustic sensor 100Y shown in FIG. 1A. In the example of FIG. 1C, the in vivo acoustic sensor 100Y includes an example embodiment of a sensor unit 110 (shown as sensor unit 110Y) and an example embodiment of electronics unit 120 (shown as electronics unit 120Y). The sensor unit 110Y includes at least one acoustic sensor 111Y to detect acoustic signals originating within the host. One or both of the sensor unit 110Y and the electronics unit 120Y are housed, fully or at least partially, in and/or coupled via a casing or housing 101Y that is deployable and biocompatible within the host's body. In some embodiments, for example, the acoustic sensor 111Y includes a transducer element 112Y coupled to a casing structure 113Y. The transducer element 112Y is able to receive a mechanical wave that emanates from a source (e.g., tissue, organ, or other) within the host's body such that the transducer element converts energy of the received mechanical wave to electrical energy, thereby producing an electrical signal corresponding to the mechanical wave. In some embodiments, for example, the transducer element 112Y includes a piezoelectric material, including but not limited to lead zirconate titanate (PZT), lead lanthanum zirconate titanate (PLZT), zinc oxide (ZnO), quartz, polyvinylidene fluoride or polyvinylidene difluoride (PVDF) aluminum nitride (AlN), scandium aluminum nitride (ScAlN), barium titanate (BaTiO₃), lead titanate (PbTiO₃), potassium niobate (KNbO₃), lithium niobate (LiNbO₃), lithium tantalate (LiTaO₃), and/or sodium tungstate (Na₂WO₄). In example embodiments where the transducer element 112Y includes PZT, the PZT can be PZT-5A, PZT-5H, or PZT-5K. In implementations of the acoustic sensor 111Y, for example, the casing structure 113Y provides a firm, inflexible material and is configured to secure and/or position the transducer element 112Y in the acoustic sensor 111Y to receive a mechanical wave for transduction to the electrical signal indicative of an in vivo acoustic signal within the body. In some embodiments, for example, the casing structure 113Y of the acoustic sensor is a hermetically sealed containment structure, which can include a metal, plastic, composite, or other material. The acoustic sensor 111Y is configured in electrical communication with the electronics unit 120Y via electrical interconnection(s) 117Y. In some embodiments, the sensor unit 110Y may include an inertial measurement unit (IMU) 115Y to monitor motion (in multiple degrees of freedom) and/or determine an orientation of the in vivo acoustic sensor 100Y. The IMU 115Y is configured in electrical communication with the electronics unit 120Y via the electrical interconnection(s) 117Y. In some embodiments of the sensor unit 110Y, for example, the IMU 115Y can include an accelerometer and/or a rotational rate sensor (e.g., gyroscope) to monitor patient motion and/or position. In some embodiments of the sensor unit 110Y, for example, the IMU 115Y can include a magnetometer. In example embodiments including the IMU 115Y, the IMU 115Y is in communication with a data processing unit of the electronics unit 120Y.

In some embodiments of the in vivo acoustic sensor 100Y, for example, the sensor unit 110Y may optionally include one or more secondary sensor(s) 119Y. For instance, in some implementations, the secondary sensor(s) 119Y may include an analyte sensor to measure a parameter (e.g., concentration) of an analyte in the region proximate the location (e.g., surrounding tissue) where the in vivo acoustic sensor 100Y is deployed within the host, such as at or proximate the heart and/or lungs. In some examples, the optional secondary analyte sensor can include, but is not limited to, a glucose sensor. Additionally or alternatively to a secondary analyte sensor, the secondary sensor(s) 119Y may optionally include a pH sensor to measure the pH level in the region proximate the location where the in vivo acoustic sensor 100Y is deployed. Additionally or alternatively to a secondary analyte sensor and/or secondary pH sensor, the secondary sensor(s) 119Y may optionally include a temperature sensor to measure the temperature in the region proximate the location where the in vivo acoustic sensor 100Y is deployed. In such cases, for example, the optional one or more secondary sensor(s) 119Y comprising the analyte sensor, the pH sensor, and/or the temperature sensor can be used to obtain data indicative of clinically-relevant conditions about the anatomic structure and/or conditions of the in vivo environment of the heart and/or lungs, such as a potential infection or inflammatory response to the implantation of the in vivo acoustic sensor 100Y. In some embodiments, for example, the optional one or more secondary sensor(s) 119Y may include an ECG sensor that includes two electrodes separated by a space that creates an electrical potential, e.g., positioned at or toward opposing ends of the housing 101Y, where the two electrodes are operable to measure an electrical signal (e.g., spike) that is indicative with the electrophysiological signals of the cardiac muscle tissue for controlling the patient's heartbeat, where the spikes give rise to the ECG signal of the patient. In some embodiments, for example, the optional one or more secondary sensor(s) 119Y can be attached to the housing 101Y of the in vivo acoustic sensor device 100Y, e.g., via weld, chemical adhesion, clip, clamp, or other attachment means.

In some embodiments of the in vivo acoustic sensor device 100Y, the housing 101Y can itself be a hermetically sealed containment structure, e.g., comprising a metal, plastic, composite, or other material. In some embodiments, for example, the housing 101Y includes titanium (Ti) container that houses the one or more sensors of the sensor unit 110Y and/or electronic components of the electronics unit 120Y, e.g., which may include but is not limited to any of sensor(s), a telemetry system, a microprocessor, memory, and/or a battery. For example, the housing 101Y is configured to shield components of the electronics unit 120Y from fluids or substances of the body when the acoustic sensor device 100Y is deployed inside the patient. As depicted in the diagram of FIG. 1C, the housing 101Y can include a containment structure that fully covers the electronics unit 120Y and at least partially covers at least one or some or all of the components of the sensor unit 110Y; whereas in some embodiments, the containment structure of the housing 101Y can fully cover at least one or some or all of the components of the sensor unit 110Y.

The electronics unit 120Y of the in vivo acoustic sensor device 100Y includes a data processing unit 121Y, an optional signal conditioning unit 123Y, a power supply 129Y, a wireless communications unit 127Y, and an electrical interface 125Y, which can include electrically conductive contact sites (e.g., pads, pins, or other contact configuration) that electrically interfaces with the electrical interconnection(s) 117Y of the sensor unit 110Y. In some embodiments of the in vivo acoustic sensor device 100Y, for example, the electronics unit 120Y can be the same or include some of the same components of the electronics unit 120X of the in vivo fluid flow sensor device 100X. The electronics unit 120Y is configured to receive and at least partially process electrical signals acquired from the acoustic sensor 111Y of the sensor unit 110Y (and signals acquired from optional IMU 115Y or optional secondary sensor(s) 119Y). For example, in some embodiments, the electrical signals are received at the corresponding contact sites of the electrical interface 125Y and provided to the data processing unit 121Y (or, optionally, first to the signal conditioning unit 123Y to improve the quality of the acquired electrical signals from the sensor unit 110Y prior to providing to the data processing unit 121Y). In such implementations, the output of the data processing unit 121Y can include raw or processed data associated with the detected data from the sensor unit 110Y, to be wirelessly transmitted to an external device by the wireless communications unit 127Y. In example embodiments of the electronics unit 120Y, the power supply 129Y can include a battery (e.g., primary or rechargeable), fuel cell or other power source to supply power to the components of the electronics unit 120Y and/or the sensor unit 110Y. In some implementations, for example, the power supply 129Y includes an ultra-low power system (e.g., operating in the micro amp or nano amp range).

In some implementations, the data processing unit 121Y to at least partially process the conditioned electrical signals to (i) produce data, e.g., in an analog or a digital form, and/or (ii) control functionality of the electronics unit 120Y and/or the sensor unit 110Y. For example, the data processing unit 121Y can be configured to manage data acquisition on data channels associated with the acoustic sensor 111Y and (optional) IMU 115Y and/or (optional) secondary sensor(s) 119Y of the sensor unit 110Y.

In some embodiments, for example, the wireless communications unit 127Y includes a wireless transmitter, receiver, and/or transceiver device, e.g., including an antenna, which is capable of communicating with an external device to communicate raw, partially-processed, or fully-processed data from the signal conditioning unit 123Y (and/or the data processing unit 121Y, discussed below). For example, the wireless communications unit 127Y can be configured to manage the communication protocol for transmission or reception via the antenna. Examples of antenna can include, but are not limited to, a whip antenna, a loop antenna, or a conformal antenna. For example, an antenna system can be attached to the housing 101Y (e.g., Ti containment structure) to enable bi-directional communication for data transfer and device management between the in vivo acoustic sensor device 100Y and one or more remote devices, e.g., various embodiments of the external remote device 130, such as a base station or phone-based interface system. An example transceiver unit can include a BLE chipset to communicate with a BLE-enabled device, e.g., a smartphone, tablet, or other external computing device. Additionally or alternatively, in some embodiments, for example, the wireless communications unit 127Y is configured as a scaffolding around the electronics unit 120Y, e.g., such as coupled to or integrated with the housing 101Y, that is structured to provide wireless communication means for the in vivo acoustic sensor device 100Y.

In some optional embodiments, for example, the optional signal conditioning unit 123Y can include a circuit including one or more filters and/or one or more amplifiers to augment the raw electrical signals detected by the acoustic sensor 111Y of the sensor unit 110Y (e.g., transducer element 112Y) to increase a signal-to-noise ratio (SNR) of the electrical signals, thereby producing data containing the signal-processed electrical signals. In some optional embodiments, the optional signal conditioning unit 123Y can include drive circuitry to produce operating electrical signals that generate electrical potentials and/or currents at the sensors, e.g., including but not limited to example embodiments of the optional secondary sensor(s) 119Y, such as analyte sensor electrode contingent(s) and/or temperature sensor contingent for operating an electrochemical sensing technique and/or electrophysiological or kinetic sensing technique to be performed at electrode(s) in some implementations of the optional secondary sensor(s) 119Y of the sensor unit 110Y.

In some embodiments of the data processing unit 121Y, for example, the data processing unit 121Y can include a processor 121AY to process data and a memory 121BY in communication with the processor 121AY to store and/or buffer data. In various embodiments, for example, the processor 121AY can include one or multiple processors, and the memory 121BY can include one or multiple memory units. For example, the processor 121AY can include a central processing unit (CPU), a microcontroller unit (MCU), a graphics processing unit (GPU), or other type of processor. For example, the memory 121BY can include and store processor-executable code, which when executed by the processor, configures the data processing unit 121Y to perform various operations, e.g., such as receiving information, commands, and/or data, processing information and data, and transmitting or providing information/data to another device. To support various functions of the data processing unit 121Y, the memory 121BY can store information and data, such as instructions, software, values, images, and other data processed or referenced by the processor 121AY. For example, various types of random access memory (RAM) devices, read only memory (ROM) devices, flash memory devices, and other suitable storage media can be used to implement storage functions of the memory 121BY. In some embodiments, the data processing unit 121Y includes an input/output (I/O) unit 121CY to interface the processor 121AY and/or memory 121BY to other modules, units or devices, e.g., associated with an external device. In some embodiments, the processor 121AY, the memory 121BY, and/or the I/O unit 121CY is in communication with the wireless communications unit 127Y, e.g., such as a transmitter (Tx) or a transmitter/receiver (Tx/Rx) unit. For example, in such embodiments, the I/O unit 121CY can interface the processor 121AY and memory 121BY with the wireless communications unit 127Y, e.g., to utilize various types of wireless interfaces compatible with typical data communication standards, which can be used in communications of the data processing unit 121Y with other devices. The data communication standards include, but are not limited to, Bluetooth, Bluetooth Low Energy (BLE), Zigbee, Medical Implant Communication Service (MICS), industrial, scientific, and medical (ISM) band, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE/5G/6G cellular communication methods, and parallel interfaces. In some implementations, the data processing unit 121Y can interface with other devices using a wired connection via the I/O unit 121CY, e.g., such as prior to implantation within the patient user or during implantation to be in data communication and/or power connection with another implanted device inside the patient user's body or wearable device worn on or attached to the outside of the patient user's body. The data processing unit 121Y can also interface, e.g., via wireless communication, with other external interfaces, sources of data storage, and/or visual or audio display devices, etc. to retrieve and transfer data and information that can be processed by the processor 121AY, stored in the memory 121BY, or exhibited on an output unit of the external receiver device 130 (e.g., smartphone, tablet, etc.) or other external device to the in vivo acoustic sensor 100Y. In some embodiments, for example, the electronics unit 120Y can include a global positioning system (GPS) to determine a location of the patient user of the in vivo acoustic sensor 100Y.

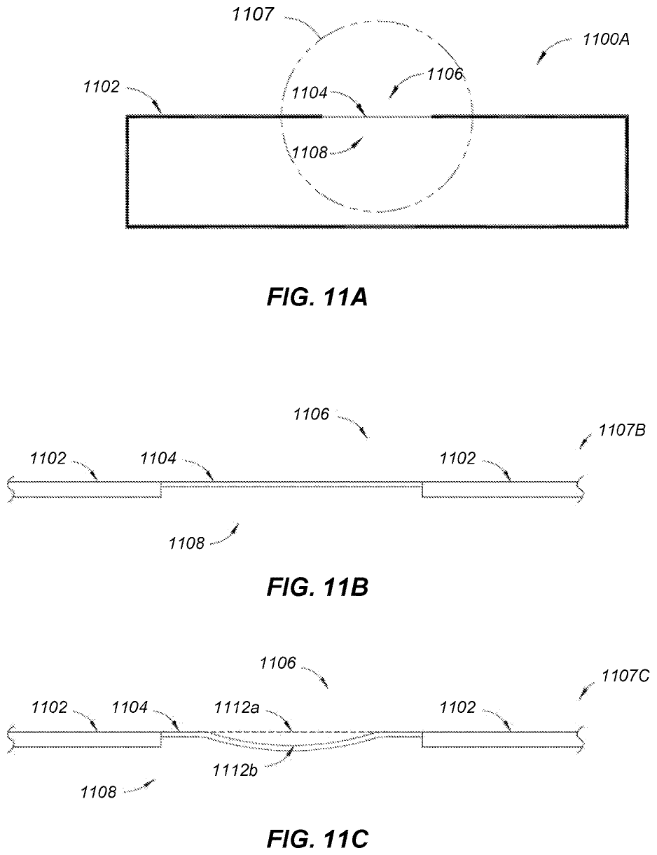

Various example embodiments of the in vivo fluid flow sensor device 100X and systems and methods involving the in vivo fluid flow sensor device 100X are described below in connection with FIGS. 2A-10B; and various example embodiments of the in vivo acoustic sensor device 100Y and systems and methods involving the in vivo acoustic sensor device 100Y are described below in connection with FIGS. 11A-22.

### In Vivo Fluid Flow Sensor Device

FIG. 2A shows a diagram depicting an example embodiment of the in vivo fluid flow sensor device 100X of FIG. 1B, shown in FIG. 2A as in vivo fluid flow sensor device 200. The in vivo fluid flow sensor device 200 includes a sensor unit comprising an ultrasound sensor assembly 211 and an ultrasound sensor assembly 212 in electrical communication with an electronics unit 220 via electrical interconnections 217 (e.g., coupled to or partially housed within linkages, such as arms, of a linkage assembly 203). The ultrasound sensor assembly 211 includes a plurality of acoustic transducer elements 213, i.e., three acoustic transducer elements 213a, 213b, 213c in this example; and the ultrasound sensor assembly 212 includes a plurality of acoustic transducer elements 214, i.e., three acoustic transducer elements 213a, 213b, 213c in this example. It is understood that this embodiment is not limited to three acoustic transducer elements for each ultrasound sensor assembly and can include less or more than the configuration shown in the diagram of FIG. 2A, e.g., including but not limited to one or more acoustic transducer element for each of the ultrasound sensor assemblies 211 and 212. The ultrasound sensor assembly 211 and the ultrasound sensor assembly 212 include a frame, casing or housing structure (not shown) to secure the acoustic transducer elements 213 and the acoustic transducer elements 214, respectively, to the arms of the linkage assembly 203 and position them in a fixed location with respect to each other.

The electronic unit 220 of the in vivo fluid flow sensor device 200 is configured to be encased by a spring connection apparatus 235 that couples to each linkage of the linkage assembly 203. The spring connection apparatus 235 provides sufficient movement of the arms of the linkage assembly 203 to couple to the intended deployment location of the ultrasound sensor assembly 211 and the ultrasound sensor assembly 212, such as across a proximal and distal side of the left or right atrium of the heart, so that the force applied by the ultrasound sensor assembly 211 and the ultrasound sensor assembly 212 upon the anatomic structure (e.g., the heart) is sufficient to secure the in vivo fluid flow sensor device 200 while not interfering with the normal function of the anatomic structure of the intended application of the in vivo fluid flow sensor device 200. For example, the spring connection apparatus 235 includes a spring, which in addition to providing compression force upon the set of linkages of the linkage assembly 203 to transfer sufficient force to facilitate and/or maintain securement of the in vivo fluid flow sensor device 200 to the target portion of the heart (e.g., left or right atrium), the spring also allows provides adequate flexibility for the device 200 to be both stable in its placement while withstanding continuous movements (load cycling) of the anatomic structure to which its attached (e.g., heart beats) without suffering damage for hundreds of millions to billions of cycles-thereby giving significant longevity to the in vivo fluid flow sensor device 200 for decades of use (e.g., 50 to 60 million heart beats a year). Moreover, the set of linkages of the linkage assembly 203 are able to adjust in all three planes (x-y, x-z, y-z) to allow for initial proper alignment of the ultrasound sensor assemblies 211, 212.

In some embodiments, for example, the in vivo fluid flow sensor device 200 can secure to the anatomic structure by a prong, screw, barb, suture, adhesive (e.g., bioinert glue), or network of grasping mechanisms (not shown) disposed via the frame or casing of the ultrasound sensor assembly 211 and the ultrasound sensor assembly 212. In some embodiments of the in vivo fluid flow sensor device 200 (not shown in FIG. 2A), the frame, casing or housing structure can include one or more openings on each linkage of the linkage assembly 203 that can allow for secondary attachment means of the device 200 to the target anatomic structure, such as an opening with an anchor site to apply a suture connecting the linkage of the linkage assembly 203 to the anatomic structure (e.g., wall of the atrium of the heart). In some embodiments, the linkage assembly 203 can include materials that provide sufficient flexibility and rigidity to allow the in vivo fluid flow sensor device 200 to deploy on the anatomic structure without such detrimental interference. For example, in some embodiments, the linkage assembly 203 can include nitinol, platinum, MP35N, or other material or combination thereof. In some embodiments, the linkage assembly 203 provides a closure to lock-on to the heart, e.g., disallowing further movement of the linkages.

FIG. 2B shows a diagram depicting another example embodiment of the in vivo fluid flow sensor device 100X of FIG. 1B, shown in FIG. 2B as in vivo fluid flow sensor device 200B. The in vivo fluid flow sensor device 200B includes a sensor unit comprising two or more sets of ultrasound sensor contingents 250 (shown in FIG. 2B as set 250A and 250B) in electrical communication with an electronics unit 220 via electrical interconnections 217 (e.g., coupled to or partially housed within sets of linkage assemblies 203A and 203B, respectively). Each set of the ultrasound sensor contingents 251 and 252 include the ultrasound sensor assembly 211 and the ultrasound sensor assembly 212 that comprise the plurality of acoustic transducer elements 213 and the plurality of acoustic transducer elements 214, respectively, such that multiple planes of acoustic signal propagation are measured at the region of interest of the anatomic structure (e.g., mitral valve of the heart). In this example, the electronics unit 220 is partially encased by two or more sets of spring connection apparatuses, shown by a spring connection apparatus 235B corresponding to the set of ultrasound sensor contingent 250B and by a spring connection apparatus 235A corresponding to the set of ultrasound sensor contingent 250A.

FIG. 2C shows a diagram depicting another example embodiment of the in vivo fluid flow sensor device 100X of FIG. 1B, shown in FIG. 2C as in vivo fluid flow sensor device 200C. The in vivo fluid flow sensor device 200C can be configured similar to the in vivo fluid flow sensor device 200 (shown previously in FIG. 2A), e.g., where the sensor unit comprises the ultrasound sensor assembly 211 and the ultrasound sensor assembly 212 in electrical communication with an electronics unit 220 via the electrical interconnections 217 (e.g., coupled to or partially housed within linkages of the linkage assembly 203). Yet, in FIG. 2C, the electronic unit 220 of the in vivo fluid flow sensor device 200C is configured to be encased by a flex connection apparatus 235C that includes a first link 236 that is able to pivotably move with respect to a second link 237, and/or vice versa, to provide adequate flexibility for the device 200C to be both stable in its placement about the anatomic structure while withstanding continuous movements (load cycling) of the anatomic structure to which its attached (e.g., heart beats) without suffering damage for hundreds of millions to billions of cycles. In some embodiments, the flex connection apparatus 235 includes a spring. In some embodiments, the first link 236 is able to move within a cavity of the second link 237, and/or vice versa.

Also, the flex connection apparatus 235C couples the sensor unit 220 to each linkage of linkage assembly 203, shown in FIG. 2C as linkages 203C. Yet, in FIG. 2C, the linkage assembly 203C include a linear arm portion proximal to the flex connection apparatus 235C and a curved arm portion distal from the flex connection apparatus and proximate to the ultrasound sensor assembly 211 and the ultrasound sensor assembly 212. In this manner, for example, the structure of the in vivo fluid flow sensor device 200C is capable of attaching to a curved geometry for an anatomic structure, such as the left or right atrium of the heart, and provide sufficient compression force for attachment with adequate flexion to mitigate load cycling. In some embodiments, for example, one or both of the linkages 203C can be used to attach (optional) additional power supply (e.g., one or more batteries), which can be in electrical connection with the electronic unit and ultrasound sensor assemblies 211 and/or 212.

Furthermore, the ultrasound sensor assembly 211 and the ultrasound sensor assembly 212 include a frame, casing or housing structure 219 to secure the acoustic transducer elements 213 and the acoustic transducer elements 214, respectively, to the linkages of the linkage assembly 203C and position them in a fixed location with respect to each other. While not shown in FIG. 2C, the frame, casing or housing structure 219 can include one or more openings to allow for secondary attachment means of the device 200 to the target anatomic structure, such as an opening with an anchor site to apply a suture and secure the to the ultrasound sensor assembly 211 and the ultrasound sensor assembly 212 at the ends of the arms of the linkage assembly 203C to the anatomic structure (e.g., wall of the atrium of the heart). In some embodiments, like the example shown in FIG. 2C, the distal ends of the arms or linkages of the linkage assembly 203C of the in vivo fluid flow sensor device 200C are angled or curved inward, e.g., which can assist in facilitating attachment and conformation to the anatomic structure upon placement as well as aid in delivery of the device 200C, e.g., via a catheter.

While not shown in FIG. 2C, the sensor unit of the in vivo fluid flow sensor device 200C may include the two or more sets of ultrasound sensor contingents 250 (such as the configuration of ultrasound sensor contingents 250A and 250B as shown in FIG. 2B) in electrical communication with an electronics unit 220C via electrical interconnections 217 (e.g., coupled to or partially housed within respective arms of the linkage assembly 203C, respectively).

FIG. 2D shows a diagram depicting another example embodiment of the in vivo fluid flow sensor device 100X of FIG. 1B, shown in FIG. 2D as in vivo fluid flow sensor device 200D. The in vivo fluid flow sensor device 200D can be configured similar to the in vivo fluid flow sensor device 200 (shown previously in FIG. 2A) and/or the in vivo fluid flow sensor device 200C (shown previously in FIG. 2C), e.g., where the sensor unit comprises example embodiments of an ultrasound sensor assembly (i.e., ultrasound sensor assembly 211D and ultrasound sensor assembly 212D) in electrical communication with the electronics unit 220 via the electrical interconnections (not shown in FIG. 2D). The electronics unit 220 is housed in connection apparatus 235. The acoustic transducer elements of the ultrasound sensor assembly 211D and 212D, respectively, are each coupled to the linkages of the linkage assembly 203D and position them in a fixed location with respect to each other. In some embodiments, for example, the connection apparatus 235D includes a hermetic package body which can carry and support a power supply, a data processing unit, and a wireless communication unit (e.g., with the antenna for transmission), e.g., enabling the power transfer to enact a charging system for a power management of functional performance. For example, mechanical force for closure by the linkages can be provided by a spring coil, deflection spring, or other method of compression to connect the ultrasound sensor assemblies 211D and 212D to their intended placement about the anatomic structure. Yet, in FIG. 2D, the linkage assembly 203D of the in vivo fluid flow sensor device 200D is configured include an anchor support 233D on each linkage of the linkage assembly 203D. The anchor supports 233D are articles that can be made from metallic or polymer material and be located on the linkage between the connection apparatus 235D and the distal portions of the linkages 239D that support the ultrasound sensor assemblies 211D and 212D. In example implementations of the device 200D, for example, the anchor supports 233D enable the linkages to be sutured or connected onto the anatomic structure (e.g., tissue or organ or vessel) by a material, e.g., such a suture silk, polymer thread, metallic suture needle, that can pass through openings 218 and bind the anchor supports 233D to the wall of the anatomic structure.

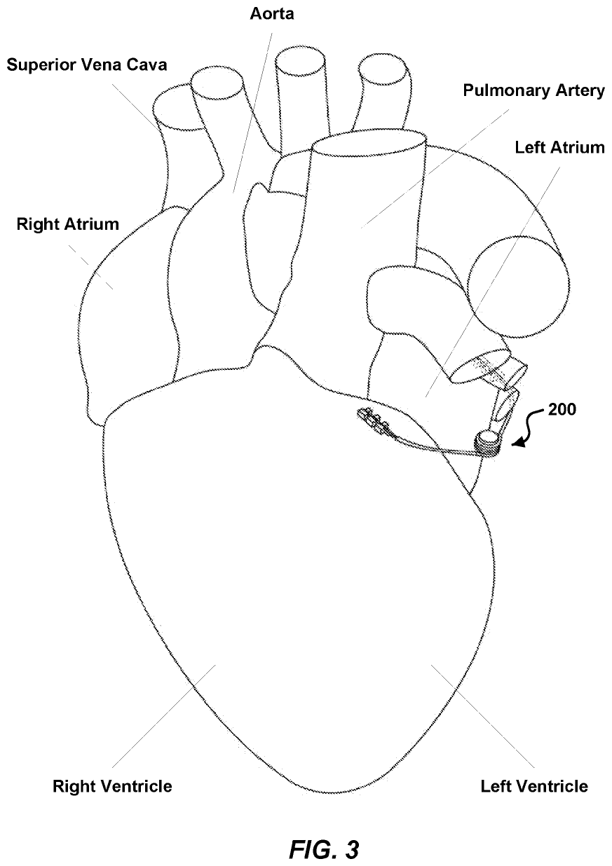

FIG. 3 shows a diagram illustrating an example embodiment of the in vivo fluid flow sensor device 200 attached to a heart of a patient user in an example implementation of the device 200. While FIG. 3 illustrates the example in vivo fluid flow sensor device 200, it is understood other embodiments of the in vivo fluid flow sensor device 100X can be attached to the heart of the patient user to assess blood flow at the target region of the heart. In this example, the in vivo fluid flow sensor device 200 is configured to assess blood flow across the mitral valve, e.g., for assessing mitral regurgitation (MR), by placing the first ultrasound sensor assembly 211 on a proximal side of the external surface of the left atrium (LA) of the heart and the second ultrasound sensor assembly 212 on a distal side of the external surface of the LA. For example, the in vivo fluid flow sensor device 200 can be secured to the LA by clamping the first and second ultrasound sensor assemblies 211, 212 onto the LA that creates a plane normal to the mitral valve for propagating the acoustic signals. As illustrated in FIG. 3, the arms of the in vivo fluid flow sensor device 200 (e.g., linkages of the linkage assembly 203 of device 200) are capable of extending about an organ, such as the LA of the heart, with a particular spring constant that can support various orientations for optimizing functional alignment with the target anatomic structure. Further, the optimal functional alignment can be preserved by the spring constant or degree of flex of the connection apparatus, which can position and secure the device to the target anatomic structure individually or in compilation with (optional) one or more secondary attachment mechanisms, e.g., such as sutures (secured at suture ports), adhesives, or anchor structures (secured at anchoring ports), which can optimize the placement against the intended area of the organ/tissue for consistent measurements (e.g., initial monitoring to establish calibration, then comparison by constant monitoring and comparison from the baseline). Also, in some implementations, for example, multiple in vivo fluid flow sensor devices 200 can be deployed at different locations of the same anatomic structure, e.g., such as the LA of the heart (as illustrated in FIG. 3) and a major blood vessel of the heart such as the vena cava or pulmonary artery or vein (not shown in FIG. 3).

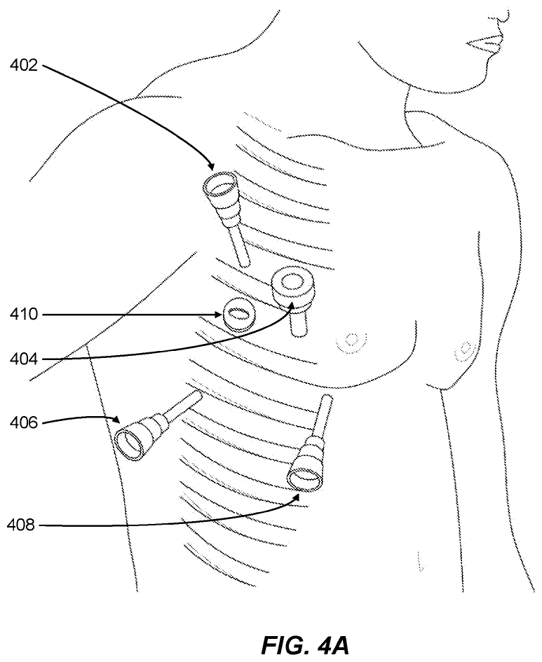

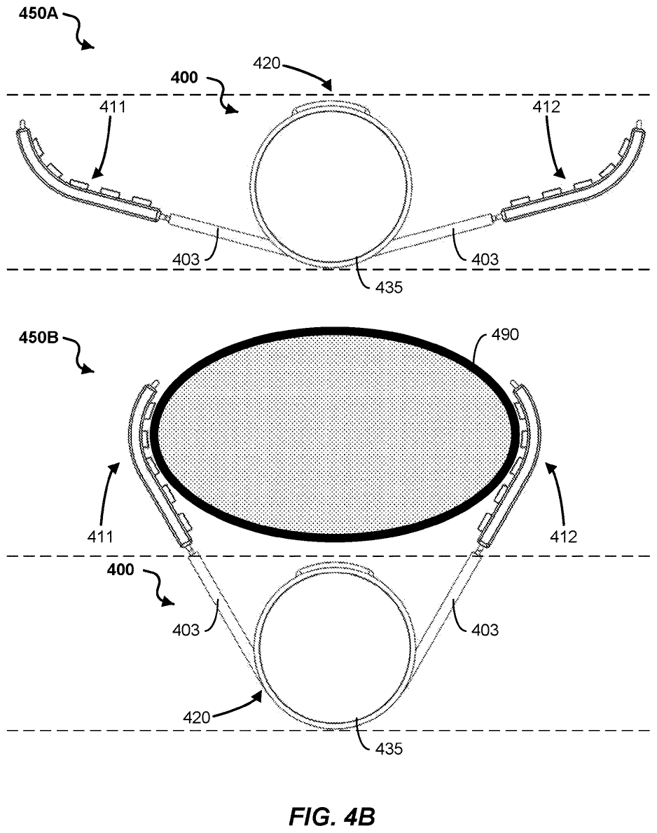

FIG. 4A shows a diagram depicting insertion sites for implanting various embodiments of the in vivo fluid flow sensor device 100X, such as the in vivo fluid flow sensor device 200 shown in FIG. 3. In some implementations, for example, the in vivo fluid flow sensor device 200 (or other embodiments of the in vivo fluid flow sensor device 100X) can be implanted and positioned onto the heart, as shown in FIG. 3, via an apical pericardial puncture and delivery of the device in the pericardial sac. The example in vivo fluid flow sensor device 200 can be attached to a mechanism for loading, carrying, and delivering into a thoracic cavity or placing on a surface of the heart (e.g., across the left atrium or right atrium) in the pericardium for monitoring fluid flow rate (e.g., forward and regurgitant flow) and other heart functions. For example, the apical pericardial implantation procedure can include using attaching the example in vivo fluid flow sensor device 200 (or other embodiments of the in vivo fluid flow sensor device 100X) in a first shape conformation to a catheter that is inserted into the patient via one of the insertion sites 402, 404, 406, 408, or 410, or other insertion site not shown in FIG. 4A, which subsequently during the implantation procedure the example in vivo fluid flow sensor device 100X can change to a second shape confirmation to secure the opposing ultrasound sensor assemblies across the target region of the heart, e.g., across the left atrium in a normal plane of the mitral valve. In some implementations, for example, the catheter can be used to communicate with the example in vivo fluid flow sensor device 100X or to recharge the power supply (e.g., power supply 129) in embodiments of the device 100X, if needed. In some implementations, embodiments of the in vivo fluid flow sensor device 100X can be implanted and positioned onto the heart outside of the pericardial sac.

In some implementations, for example, the in vivo fluid flow sensor device 100X can be inserted in a first phase (first conformation), e.g., by a physician user such as a thoracic surgeon, into a patient user of the device through the patient user's chest cavity by a main sternotomy or thoracotomy procedure. After insertion, the in vivo fluid flow sensor device 100X can undergo a second phase (second conformation) for placement about the heart, e.g., at the transition region between the atrium to ventricle on the left side, or at the right side in same position. The in vivo fluid flow sensor device 100X may, on its own, secure to the site of the anatomic structure placed by the surgeon, e.g., based on compression and flexion properties of the connection apparatus and/or arms (of the linkage assembly 103B), and/or may be anchored in place with suture or metal anchor, e.g., performed by the physician user.

FIG. 4B shows diagrams illustrating example shape conformations of example embodiments of the in vivo fluid flow sensor device, in accordance with the present technology, for an implantation process near a target anatomic structure and a deployment process to secure to the target anatomic structure. Diagram 400A shows another example embodiment of the in vivo fluid flow sensor device 100X of FIG. 1B, shown in FIG. 4B as in vivo fluid flow sensor device 400, having arms of linkage assembly 403 spread outward from connection apparatus 435 to be in a first shape conformation, e.g., for an insertion or implantation process, such as implemented by a catheter. For example, in some implementations, the in vivo fluid flow sensor device 400 can be inserted into the patient's body (e.g., through a port through the ribs like the insertion site(s) shown in FIG. 4A) to approach the region where the target anatomic structure is located, e.g., such as the heart. Prior to the insertion process, the patient can be prepared by undergoing an imaging session (e.g., CT scan of the target area), which can be used in assisting the insertion.

Diagram 400B shows the in vivo fluid flow sensor device 400 in a second shape conformation, having arms of linkage assembly 403 spanning inward toward a centerline through the connection apparatus 435, e.g., for a deployment process to attach to anatomic structure 490. The in vivo fluid flow sensor device 400 can be configured similar to the in vivo fluid flow sensor device 200 (shown previously in FIG. 2A), e.g., where the sensor unit comprises the ultrasound sensor assembly 411 and the ultrasound sensor assembly 412 in electrical communication with an electronics unit 420 via the electrical interconnections (not shown). For example, during or after attachment of the in vivo fluid flow sensor device 400 to the anatomic structure 490, the ultrasound sensor assemblies 411 and 412 on the distal portions of the linkages 403 are able to be oriented about the intended measurement site (e.g., by the insertion instrument prior to its withdrawal), such that a line of site of the acoustic transducers of the ultrasound sensor assemblies 411 and 412 can be established (e.g., via transmission and reception of acoustic signals) for contact placement and/or calibration measurements. Also, for example, prior to withdrawal of the insertion instrument, the in vivo fluid flow sensor device 400 can be tested for wireless data communication with a remote device (e.g., remote computing device 130A and/or remote base station 130b). In some implementations, for example, sutures or other securement techniques (e.g., adhesive or other) can be used to further secure the in vivo fluid flow sensor device 400.

In some example embodiments of the ultrasound sensor assembly or assemblies for various embodiments of the in vivo fluid flow sensor device 100X of FIG. 1B, the transducer arrays are designed to spatially configure a single transmitter with at least two receivers interlaying and exchanging the receivers and transmitter, such that a singular- or multi-field of ultrasonic energy is displaced about the heart chamber (e.g., atrium or ventricle), e.g., for detecting Q-wave flow pattern(s) or disruption(s) in the flow pattern(s). By detecting the Q-wave flow pattern(s) or disruption(s) in the flow pattern(s), this data that can be interrogated and mapped to regurgitation of the blood flowing through and backflowing back through the chamber valve, which indicates wall/chamber disfunction (e.g., and can be related to electro-signal disruption for heart pulsation, and/or coagulation of static blood). Also a mechanical analysis of the chamber diametrical change can be calibrated and detected for change in use by the exemplary ultrasonic sensor assemblies 511 and/or 112, and therefore a tool for predicting heart structural-changes, e.g., such as due to water absorption by heart cells that can lead to the saturation or super saturation indicating disfunction of cell analyte removal, such as water, electrolytes, and/or cellular waste, which can result to CHF in a patient.

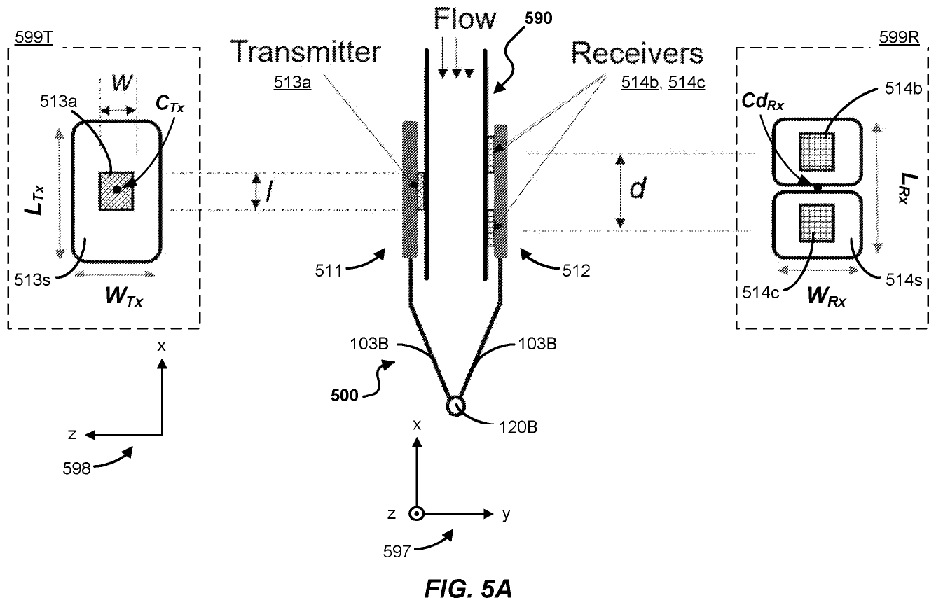

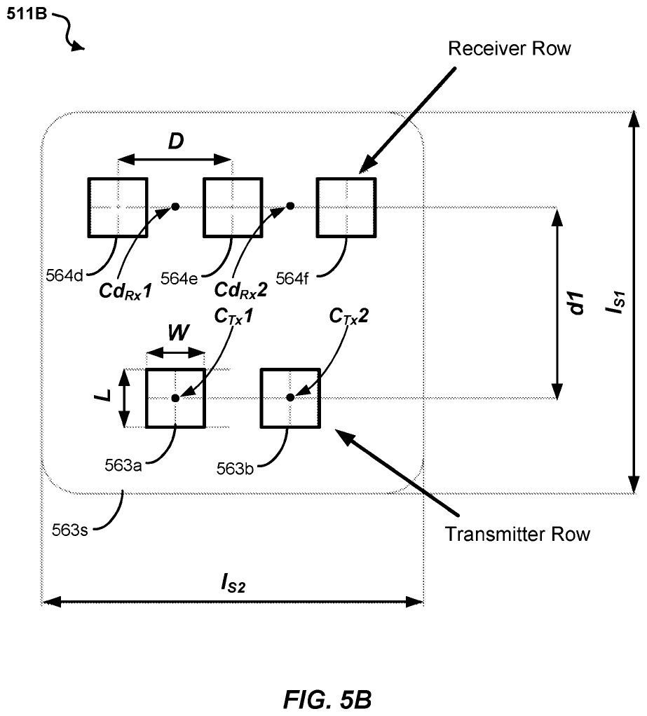

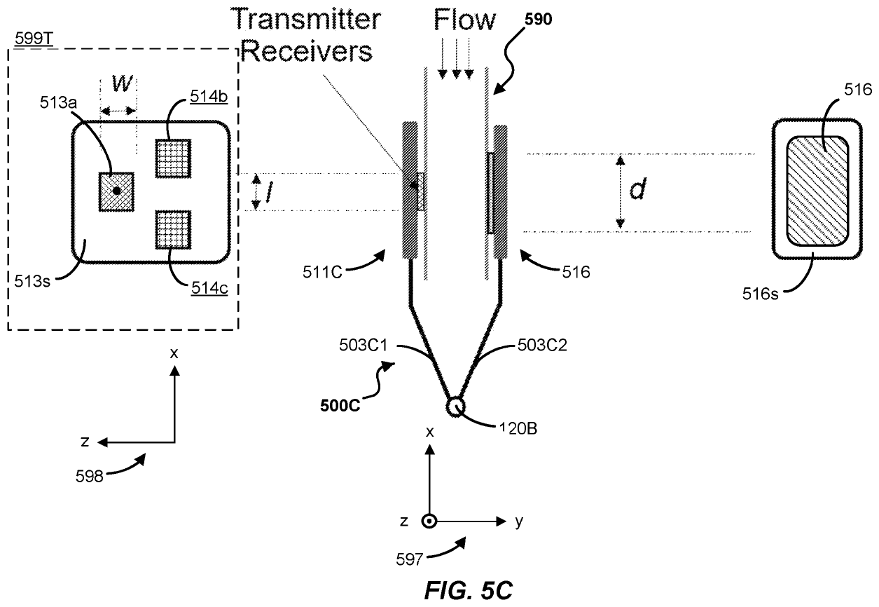

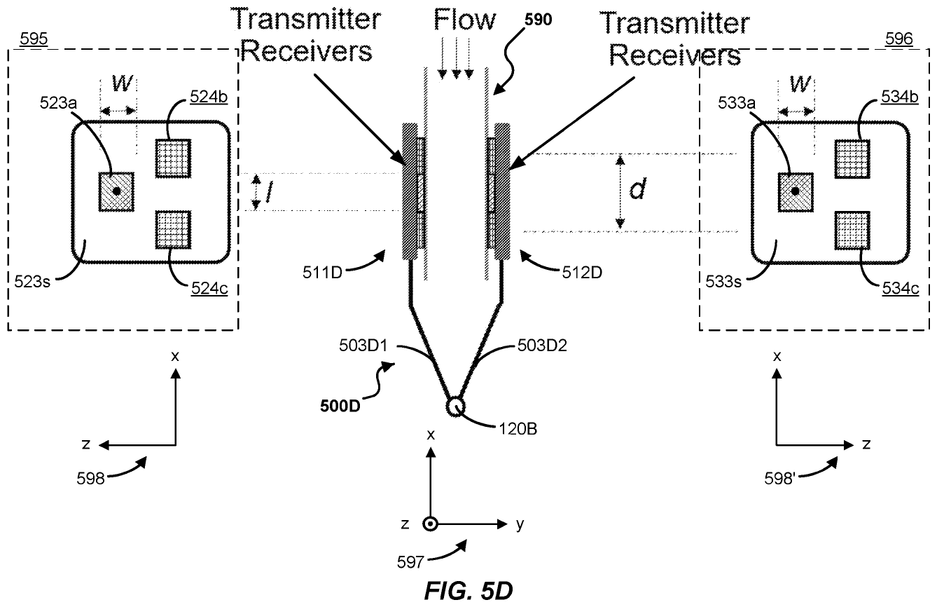

FIG. 5A shows a diagram illustrating an example implementation of the in vivo fluid flow sensor device 100X of FIG. 1B, shown in FIG. 5A as in vivo fluid flow sensor device 500 attached to an anatomic structure 590 (e.g., an atrium or ventricle of the heart or blood vessel flowing blood into or out of the heart), depicting an example embodiment of an acoustic transducer configuration in accordance with the present technology. The example in vivo fluid flow sensor device 500 includes an embodiment of the electronics unit 120X coupled to an embodiment of the linkage assembly 103B, having two opposing arms, where at their distal ends is a first ultrasound sensor assembly 511 disposed on a first arm and a second ultrasound sensor assembly 512 disposed on a second arm. The first ultrasound sensor assembly 511 includes at least one ultrasound transmitter (Tx) transducer 513a, and the second ultrasound sensor assembly 512 includes at least two ultrasound receiver (Rx) transducers 514b and 514c. While the drawing in FIG. 5A graphically depicts a single Tx transducer 513a on one arm of the example linkage assembly 103B and two Rx transducers 514b and 514c on the opposing arm of the example linkage assembly 103B, in some example embodiments, an additional Tx transducer element or additional Tx transducer elements can be included on the first ultrasound sensor assembly 511; and, in some example embodiments, an additional Rx transducer element or additional Rx transducer elements can be included on the second ultrasound sensor assembly 512. Also, for example, in some embodiments, the first ultrasound sensor assembly 511 can include two or more Rx transducer elements (e.g., at least the two Rx transducers 514b and 514c); and, for example, in some embodiments, the second ultrasound sensor assembly 512 can include one or more Tx transducer elements (e.g., at least the one Tx transducer 513a).

In the example shown in FIG. 5A, the in vivo fluid flow sensor device 500 provides a single-side acoustic detection system, where one or more Tx transducer(s) 513a are configured on one side (i.e., arm of the linkage assembly) of the device 500 to transmit acoustic signals (e.g., pulses, waveforms, etc.), and where at least two Rx transducers 514b, 514c are configured on either (i) the opposing side (i.e., other arm of the linkage assembly as the Tx transducer(s) 513a) of the device 500 to receive acoustic signals that correspond to the transmitted acoustic signals after propagating through the fluid in the anatomic structure 590, subject to reflections, refractions, or other propagation changes due to the fluid flow, or (ii) the same side (i.e., the same arm of the linkage assembly as the Tx transducer(s) 513a) of the device 500 to receive acoustic signals that correspond to the transmitted acoustic signals after propagating through the fluid in the anatomic structure 590, subject to reflections, refractions, or other propagation changes due to the fluid flow, and reflected back by a reflector structure (not shown in FIG. 5A; shown later) on the opposing side.

In some example embodiments, as shown in FIG. 5A, the acoustic transducers on the first and second ultrasound sensor assemblies 511 and 512 are configured such that the Tx transducer 513a is positioned on/about another portion (e.g., opposing portion) of the anatomic structure 590 from the Rx transducers 514b, 514c, where the center (C_{Tx}) of the Tx transducer 513a is aligned with the center between the distance (d) separating the centers of the Rx transducers 514b, 514c, i.e., (Cd_{Rx}). The spatial alignment of C_{Tx} of the Tx transducer 513a and the Cd_{Rx} between the Rx transducers 514b, 514c is configured to minimize interference of the acoustic energy during operation of the ultrasound sensor assembly or assemblies. Inset box 599T shows that the center of the Tx transducer 513a, C_{Tx}, is the midpoint of the length (I), which defines the length of a side of the Tx transducer 513a in that dimension, e.g., along the x-axis as shown by example coordinate map 598 in FIG. 5A. In a perpendicular dimension, e.g., along the z-axis, the center of the Tx transducer 513a, C_{Tx}, is also the midpoint of the width (w). Inset box 599R shows that the center between the distance (d) separating the centers of the Rx transducers 514b, 514c, i.e., (Cd_{Rx}), is based on the relative lengths of the sides of the Rx transducers 514b, 514c in that dimension, e.g., along the x-axis as shown by example coordinate map 598. Notably, the fluid flow direction is in the direction of the x-axis of the coordinate map 598 corresponding to the ultrasound sensor assemblies shown in insets 599T and 599R, as well as the coordinate map 597 corresponding to the diagram of the device 500 secured to the anatomic structure 590.

As illustrated in inset box 599T, in some example embodiments, the Tx transmitter 513a can be coupled to a backing or substrate 513s that can attach to the inner surface of the arm of the linkage assembly 103B. For example, the substrate 513s can provide a heat sync for management of thermal generation by the acoustic transducer(s). Similarly, inset box 599R illustrates an example embodiment of the Rx transmitters 514b, 514c coupled to a backing or substrate 514s, which can attach to an inner surface of the arm of the linkage assembly 103B. For example, in some embodiments, one or some of the Rx transmitters 514b, 514c can be disposed on a single substrate 514s or individual substrates 514s, in various combinations. Similarly, for example, the substrate 514s can provide a heat sync for management of thermal generation by the acoustic transducer(s).

In some embodiments, for example, the Tx transducer 513a is configured to have a 4 mm² transducer area (e.g., 2 mm×2 mm) or have a 41 mm² transducer area (e.g., 2 mm diameter) for transmission of the acoustic signals. It is understood that the width, length, or diameter (or any size dimension) of the Tx transducer 513a may be greater or less than 2 mm; for example, the size dimension (e.g., width, length, diameter, etc.) of the Tx transducer 513a can be between 1 mm to 4 mm. In some embodiments, for example, each of the Rx transducers 514b, 514c is configured to each have a 4 mm² transducer area (e.g., 2 mm×2 mm) or have a 4π mm² transducer area (e.g., 2 mm diameter) for reception of the acoustic signals. It is understood that the width, length, or diameter (or any size dimension) of the Rx transducers 514b, 514c may be greater or less than 2 mm; for example, the size dimension (e.g., width, length, diameter, etc.) of the Rx transducer 514b, 514c can be between 1 mm to 4 mm.

In some embodiments, for example, the Tx transducer 513a and the Rx transducers 514b, 514c are arranged to be positioned in plane (0 degrees) for line of site and wave detection, such that the Rx transducers 514b, 514c is detecting and responding to the acoustic signal from the Tx transducer 513a that have propagated across the anatomic structure 590 and are effected by fluid flow (e.g., blood flow in the heart or blood vessel leading to or from the heart). Yet, in some embodiments, for example, the Tx transducer 513a and the Rx transducers 514b, 514c are arranged to be positioned (e.g., rotated) up to 90 degrees, with respect to each other, out of plane for line of site and wave detection. Similarly, in some embodiments, for example, the Tx transducer 513a and/or the Rx transducers 514b, 514c can be tilted up to 20 degrees, with respect to each other, out of plane for line of site and wave detection.

In some implementations, for example, the substrate 513s for the Tx transducer 513a can be configured to have width (W_{Tx}) of 5 mm and a length (L_{Tx}) of 10 mm. Also, for example, in some implementations, the substrate 514s for the Rx transducers 514b, 514c can be configured to have width (W_{Rx}) of 5 mm and a length (L_{Rx}) of 10 mm.

FIG. 5B shows a diagram depicting an example arrangement of acoustic transducers on an ultrasound sensor assembly 511B for an example embodiment of the in vivo fluid flow sensor device 100X, such as for one or both of the first ultrasound sensor assembly 511 and the second ultrasound sensor assembly 512 of the in vivo fluid flow sensor device 500 shown in FIG. 5A. The ultrasound sensor assembly 511B includes a plurality of acoustic transducer elements 563, i.e., two acoustic transducer transmitter elements 563a and 563b in this example, and a plurality of acoustic transducer elements 564, i.e., three acoustic transducer receiver elements 564d, 564e, and 564f in this example. It is understood that this embodiment is not limited to two acoustic transducer transmitter elements and three acoustic transducer receiver elements for the ultrasound sensor assembly 511B, which can include less or more than the configuration shown in the diagram of FIG. 5B. In this example, the placement of the transmitter elements 563a, 563b are in a row along a single direction, and the placement of the receiver elements 564d, 564e, 564f are along a different row above or below the transmitter row, which is positioned at a distance (d1) with respect to the center of the transmit elements and receiver elements. The configuration of the transmitter elements 563a, 563b and the receiver elements 564d, 564e, 564f is based on the line-of-site transmission and reception (LOSTR) for the operation of the acoustic transducers in the array. In the example embodiment shown in FIG. 5B, for a single transmitter element corresponding to at least two receiver transducers (e.g., transmitter element 563a corresponding to receiver elements 564d, 564e), the center (C_{Tx1}) of the Tx transducer 563a is aligned with the center between the distance (d) separating the centers of the Rx transducers 564d, 564e, i.e., (Cd_{Rx1}), such that the spatial alignment of C_{Tx1} of the Tx transducer 563a and the Cd_{Rx1} between the Rx transducers 564d, 564e is configured to minimize interference of the acoustic energy during operation of that transmitter-receivers group of the ultrasound sensor assembly 511B. Similarly, for example, the center (C_{Tx2}) of the Tx transducer 563b is aligned with the center between the distance (d) separating the centers of the Rx transducers 564e, 564f, i.e., (Cd_{Rx2}), such that the spatial alignment of C_{Tx2} of the Tx transducer 563b and the Cd_{Rx2} between the Rx transducers 564e, 564f is configured to minimize interference of the acoustic energy during operation of that transmitter-receivers group of the ultrasound sensor assembly 511B.

Notably, for example, some example embodiments of the ultrasound sensor assembly 511B can configure the transmitter row and the receiver row to be changed with a combination of transmitter elements and receiver elements integrated together in the same row. For example, in some implementations, the Tx transducer 563a can be configured to transmit one or more acoustic signals to which acoustic signals propagated through the anatomic structure 590 are receivable by the Rx transducers 564d and 564e, and concurrently or subsequently the transducer 564e can be configured to transmit one or more acoustic signals to which acoustic signals propagated through the anatomic structure 590 are receivable by the transducers 563a and 563b.

In some embodiments, for example, the transmitter elements 563a, 563b can be configured to have a 2.25 mm² transducer area (e.g., 1.5 mm×1.5 mm) or have a 2.25π mm² transducer area (e.g., 1.5 mm diameter) for transmission of the acoustic signals; and the receiver elements 564d, 564e, 564f can be configured to have a 2.25 mm² transducer area (e.g., 1.5 mm×1.5 mm) or have a 2.25π mm² transducer area (e.g., 1.5 mm diameter) for reception of the acoustic signals (e.g., transmitted from an opposing side array on the opposing arm of the linkage assembly of the device 500). It is understood that the width, length, or diameter (or any size dimension) of the transmitter and/or receiver transducer elements 563a, 563b and/or 564d, 564e, 564f may be greater or less than 1.5 mm. In some embodiments of the ultrasound sensor assembly 511B, the distance (d1) between the transmitter row and the receiver row, i.e., the centerline-to-centerline distance, can be substantially 5 mm, e.g., to achieve an optimal LOSTR (e.g., as a minimal distance for minimization of interference). For this example configuration of the ultrasound sensor assembly 511B, the distance separation (d1) has been optimized to ensure no cross reflection, which would be similar as a ghost signal or a signal disturbance that can create a false reading. The carrier for the transmitter and receiver can act as a coupler to the electronic communication to the processer and the power source. In some embodiments, for example, the one or more of the transmitter elements 563a and 563b can be rotated up to 90-degrees out of line-of-site in the same plane with respect to the corresponding at least two of the receiver elements 564d, 564e, and 564f, and vice versa.

The ultrasound sensor assembly 511B optionally includes a backing or substrate 563s, which can be used to attach the ultrasound sensor assembly 511B to an arm of a linkage assembly of the device 500. For example, the substrate 563s can provide a heat sync for management of thermal generation by the acoustic transducer(s) (e.g., acoustic transducers 563a, 563b, 564d, 564e, 564f). In some embodiments, for example, the substrate 563s can be configured to have a first side length (l_{S1}) of 10 mm (perpendicular to the transmitter and receiver rows) and a second side length (I_{S2}) of 10 mm (parallel to the transmitter and receiver rows).

In some implementations, for example, the ultrasound sensor assembly 511B can be employed in an example single-side acoustic detection system for an in vivo fluid flow sensor device 100X, where one or more Tx transducer(s) are configured on one side (i.e., arm of the linkage assembly) to transmit acoustic signals (e.g., pulses, waveforms, etc.) across the anatomic structure such that at least two Rx transducers corresponding to Tx transducer are configured on the opposing side (i.e., other arm of the linkage assembly) to receive acoustic signals that correspond to the transmitted acoustic signals after propagating through the fluid in the anatomic structure, subject to reflections, refractions, or other propagation changes due to the fluid flow. Yet, some implementations, for example, the ultrasound sensor assembly 511B can be employed in an example double-side acoustic detection system for an in vivo fluid flow sensor device 100X, where a first set of one or more Tx transducer(s) are configured on one side (i.e., arm of the linkage assembly) across the anatomic structure from a first set of the at least two corresponding receiver elements that operate (e.g., transmit and receive) concurrently with a second set of one or more Tx transducer(s) on an opposing arm across the anatomic structure from a second set of the at least two corresponding receiver elements. An example of a double-side acoustic detection system is illustrated later in FIG. 5D.

FIG. 5C shows a diagram illustrating an example implementation of the in vivo fluid flow sensor device 100X of FIG. 1B, shown in FIG. 5C as in vivo fluid flow sensor device 500C attached to the anatomic structure 590 (e.g., an atrium or ventricle of the heart or blood vessel flowing blood into or out of the heart), depicting an example embodiment of an acoustic transducer configuration in accordance with the present technology. The example in vivo fluid flow sensor device 500C includes an embodiment of the electronics unit 120X coupled to an embodiment of the linkage assembly 103B, having two opposing arms, where at a distal end of one of the arms 503C1 is an ultrasound sensor assembly 511C, and at a distal end of the opposing arm 503C2 is a reflector 516. In some embodiments, for example, the reflector 516 can be configured as a specular reflector, having a substantively flat surface, a substantively smooth surface, and a boundary/area sized larger than the acoustic signal(s) wavelength(s). Whereas, in some embodiments, for example, the reflector 516 can be configured as a non-specular reflector, having a non-flat surface, and/or a rough or irregular surface texture, and/or a boundary/area sized smaller than the acoustic signal(s) wavelength(s). And, in some embodiments, the reflector 516 can be configured to have a specular reflector portion and a non-specular reflector portion. In various embodiments, for example, the reflector 516 can be configured in shape and size to the contour of the anatomic site (e.g., organ or vessel), in compilation with the focal length reflection for optimal energy response. Example materials of the reflector 516 can include, but is not limited to, mylar (e.g., on a substrate); a precious-metal such as silver, platinum, gold, palladium (e.g., vacuum-deposited precious metal); or a standard metal such as silver, titanium, nitinol or a stainless steel (e.g., polished for the wavelength), or a combination thereof.

The ultrasound sensor assembly 511C includes at least one ultrasound transmitter (Tx) transducer 513a, and at least two ultrasound receiver (Rx) transducers 514b and 514c. In this configuration, the in vivo fluid flow sensor device 500 provides a single-side acoustic detection system, where one or more Tx transducer(s) 513a are configured in an array with at least two Rx transducers 514b, 514c on one side (i.e., arm of the linkage assembly) of the device 500C to transmit acoustic signals (e.g., pulses, waveforms, etc.) that propagate across the anatomic structure 590, are affected by fluid flow through the anatomic structure 590, reflect from the reflector 516 configured on the opposing side (i.e., other arm of the linkage assembly) of the device 500C, and are received as acoustic signals that correspond to the transmitted acoustic signals.

In some example embodiments, these acoustic transducers on the ultrasound sensor assembly 511C can be configured with exemplary sizes, spacings, materials, and structures as described for the example acoustic transducers for the first and second ultrasound sensor assemblies 511 and 512, respectively, in connection with FIGS. 5A and 5B.

Example implementations of the in vivo fluid flow sensor device 500 were performed in an experimental benchtop apparatus designed to simulate blood flow through a chamber or tube akin to the anatomic structure 590, which tested properties of the received acoustic signals. Table 1 describes the function of frequency and sensitivity of an example single-side acoustic detection systems, i.e., at least one transmitter configured on an opposing arm of the linkage assembly to at least two receivers, e.g., exemplified by the example embodiments of the in vivo fluid flow sensor device 500 incorporating the ultrasound sensor assemblies 511, 512 shown in FIG. 5A, 511B shown in FIG. 5B, and/or 511C shown in FIG. 5C.

TABLE 1

| X | 3 MHz | | 6 MHz | | 9 MHz | |
|---|---|---|---|---|---|---|
| (mm) | ΔΦ | Δt | ΔΦ | Δt | Δ Φ | Δt |
| 1 | 0.18 | 164.1 | 0.35 | 164.1 | 0.53 | 164.1 |
| 2 | 0.35 | 328.1 | 0.71 | 328.1 | 1.06 | 328.1 |
| 3 | 0.53 | 492.2 | 1.06 | 492.2 | 1.59 | 492.2 |
| 4 | 0.71 | 656.3 | 1.42 | 656.3 | 2.13 | 656.2 |
| 6 | 1.06 | 984.4 | 2.13 | 984.4 | 3.19 | 984.2 |
| 8 | 1.42 | 1312.5 | 2.84 | 1312.5 | 4.25 | 1312.5 |

Table 1 depicts the relationship between Flow Sensitivity (Δφ/Δt) with the example frequency (e.g., 3 MHz, 6 MHz, and 9 MHz) and the distance (mm) between the transmit and receive acoustic transducers for single-side transducer configuration. The units of sensitivity, Δφ, are (deg/[l/min]); and the units of duration or period of function, Δt, are (ps/[l/min]).

FIG. 5D shows a diagram illustrating an example implementation of the in vivo fluid flow sensor device 100X of FIG. 1B, shown in FIG. 5D as in vivo fluid flow sensor device 500D attached to the anatomic structure 590 (e.g., an atrium or ventricle of the heart or blood vessel flowing blood into or out of the heart), depicting an example embodiment of an acoustic transducer configuration in accordance with the present technology. The example in vivo fluid flow sensor device 500D includes an embodiment of the electronics unit 120X coupled to an embodiment of the linkage assembly 103B, having two opposing arms, where at their distal ends is a first ultrasound sensor assembly 511D disposed on a first arm and a second ultrasound sensor assembly 512D disposed on a second arm. The first ultrasound sensor assembly 511D, shown in inset 595, includes at least one ultrasound transmitter (Tx) transducer 523a; and the second ultrasound sensor assembly 512D, shown in inset 596, includes at least two ultrasound receiver (Rx) transducers 534b and 534c that correspond to the Tx transducer 523a. In the example embodiment shown in FIG. 5D, the second ultrasound sensor assembly 512D also includes at least one ultrasound transmitter (Tx) transducer 533a, and the first ultrasound sensor assembly 511D also includes at least two ultrasound receiver (Rx) transducers 524b and 524c that correspond to the Tx transducer 533a.

In the example shown in FIG. 5D, the in vivo fluid flow sensor device 500D provides a double-side acoustic detection system for an in vivo fluid flow sensor device 100X, where a first set of one or more Tx transducer(s) (e.g., Tx transducer 523a) are configured on one side (i.e., arm of the linkage assembly) across the anatomic structure 590 from a first set of the at least two corresponding receiver elements (e.g., Rx transducers 534b, 534c) that operate (e.g., transmit and receive) concurrently with a second set of one or more Tx transducer(s) (e.g., Tx transducer 533a) on an opposing arm across the anatomic structure 590 from a second set of the at least two corresponding receiver elements (e.g., Rx transducers 524b, 524c).

In some example embodiments, these acoustic transducers on the first ultrasound sensor assembly 511D and/or the second ultrasound sensor assembly 512D can be configured with exemplary sizes, spacings, materials, and structures as described for the example acoustic transducers for the first and second ultrasound sensor assemblies 511 and 512, respectively, in connection with FIGS. 5A and 5B.

Example implementations of the in vivo fluid flow sensor device 500D were performed in an experimental benchtop apparatus designed to simulate blood flow through a chamber or tube akin to the anatomic structure 590, which tested properties of the received acoustic signals. Table 2 describes the function of frequency and sensitivity of an example double-side acoustic detection systems, e.g., exemplified by the example embodiment of the in vivo fluid flow sensor device 500D incorporating the ultrasound sensor assemblies 511D, 512D shown in FIG. 5D.

TABLE 2

| | | | |
|---|---|---|---|
| X | 3 MHz | 6 MHz | 9 MHz |

| (mm) | ΔΦ | Δt | ΔΦ | Δt | Δ Φ | Δt |
|---|---|---|---|---|---|---|
| 1 | 0.09 | 82.0 | 0.18 | 82.0 | 027 | 82.0 |
| 2 | 0.18 | 164.1 | 0.35 | 164.1 | 0.53 | 164.1 |
| 3 | 0.27 | 246.1 | 0.53 | 246.1 | 0.08 | 264.1 |
| 4 | 0.35 | 328.1 | 0.71 | 328.1 | 1.06 | 328.1 |
| 6 | 0.53 | 492.2 | 1.06 | 492.2 | 1.59 | 492.2 |
| 8 | 0.71 | 656.3 | 1.42 | 656.3 | 2.13 | 656.3 |

Table 2 depicts the relationship between Flow Sensitivity (Δφ/Δt) with the example frequency (e.g., 3 MHz, 6 MHz, and 9 MHz) and the distance (mm) between the transmit and receive acoustic transducers for double-side transducer configuration, i.e., a first set of at least one transducer element on an opposing arm across the anatomic structure from a first set of the at least two corresponding receiver elements that operate (e.g., transmit and receive) concurrently with a second set of at least one transducer element on an opposing arm across the anatomic structure from a second set of the at least two corresponding receiver elements. The units of sensitivity, Δφ, are (deg/[l/min]); and the units of duration or period of function, At, are (ps/[l/min]).

The example data of Table 2 demonstrates a dampening effect using two opposing transmitters (e.g., Tx transducer 523a of first ultrasound sensor assembly 511D and Tx transducer 533a of second ultrasound sensor assembly 512D) configured to concurrently transmit acoustic signals in directions opposing each other, which is approximately half of the flow sensitivity as compared to the example data of Table 1, which is based on a ultrasound sensor assembly single-sided transmitter transducer arrangement that transmit toward a reflector with the at least two receiver transducers on the same side as the transmitter transducer, or with the at least two receiver transducers on the opposite side of the anatomic structure as the transmitter transducer.

In some example implementations of the various embodiments of the in vivo fluid flow sensor device 100X, the device can be operated to transmit and receive acoustic signals (e.g., ultrasound signals) in Doppler mode. In contrast with typical ultrasound techniques to interrogate a stationary object, Doppler ultrasound can be used to characterize fluid flow properties. For example, when a target of interest (such as blood cells in blood flow through an anatomic structure) is moving with respect to an ultrasound signal, it encounters more oscillations per unit time than a stationary equivalent, such that the frequency of the reflected wave is increased; and when the target of interest is moving away from the ultrasound signal, the frequency of the reflected signal is reduced. The Doppler effect can be used to measure the velocity of blood flow through the anatomic structure.

The example embodiments of the in vivo fluid flow sensor device 100X can be positioned at an angle (θ) with respect to the blood flow direction within the anatomic structure 590, such that the frequency shift to be measured by the Doppler mode is (f_{R}-f_{T})=2 v f_{T} cos (θ)/c, where f_{T} is the frequency of the transmitted acoustic signal, f_{R} is the frequency of the acoustic signal received, and c is the velocity of the acoustic signal of a given wavelength transmitted in the medium, and v is the velocity of the acoustic signal moving toward the acoustic transducer receiving the signal.

For example, the in vivo fluid flow sensor device 100X is able to use the Doppler technique in conjunction with mass flow calculations of the blood through the anatomic structure such that the body mass flow in a confidence interval is equal to the density (known blood) and the diametric change (used to calculate area), which enables the determination of velocity. And, from velocity and diameter, the device 100X is able to determine Q of fluid flow. Furthermore, the in vivo fluid flow sensor device 100X is able to measure planar dimensions of the target anatomic structure based on the determinable changes in fluid flow with respect to a baseline measurement, e.g., since increased fluid amount (volume) typically causes the walls of the anatomic structure (e.g., major blood vessel) to expand, i.e., an increase in the diameter of the walls.

In some example implementations of the various embodiments of the in vivo fluid flow sensor device 100X, the device can be operated to transmit and receive acoustic signals (e.g., ultrasound signals) to create ultrasound images of the target anatomic structure, e.g., using A-mode, B-mode, and/or C-mode ultrasound data acquisition techniques. In some examples, embodiments of the in vivo fluid flow sensor device 100X may be used in A-Mode ultrasound acquisition to obtain a one-dimensional presentation from the transmitted and received acoustic signals waves in the direction that the ultrasound transducer assembly is oriented, e.g., where one axis represents a depth and a perpendicular axis represents an amplitude. In some examples, embodiments of the in vivo fluid flow sensor device 100X may be used in B-Mode, also referred to as 2D mode, to obtain a two-dimensional presentation from the transmitted and received acoustic signals waves, e.g., based on the angle that the ultrasound transducer assembly is positioned with respect to the anatomic structure. In some examples, embodiments of the in vivo fluid flow sensor device 100X may be used in C-Mode to obtain both a range of depth from A-Mode and 2D information from B-Mode.

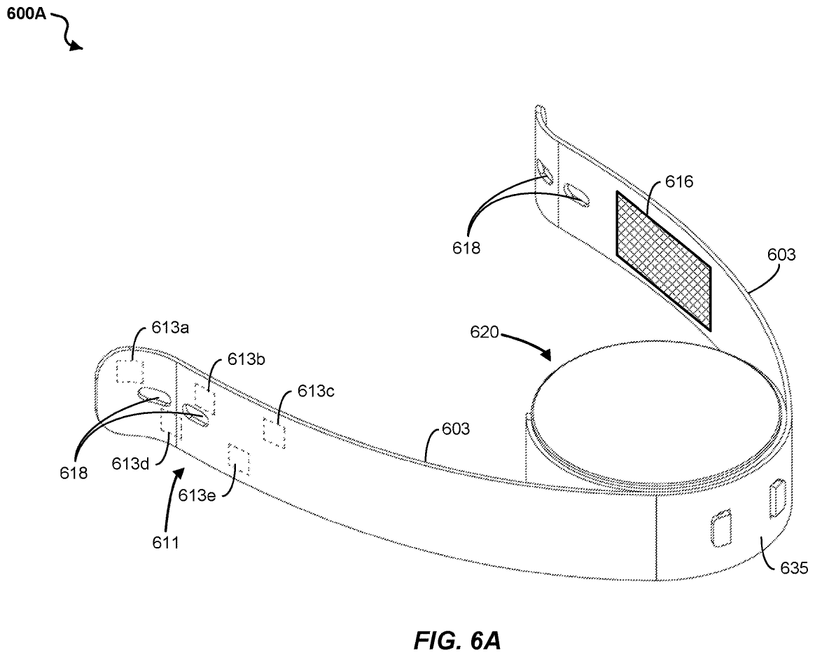

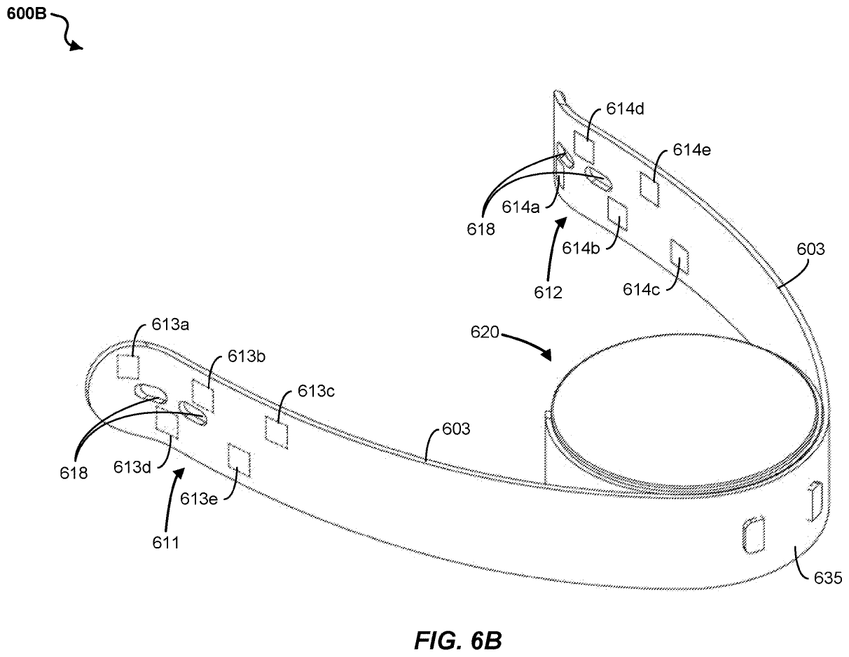

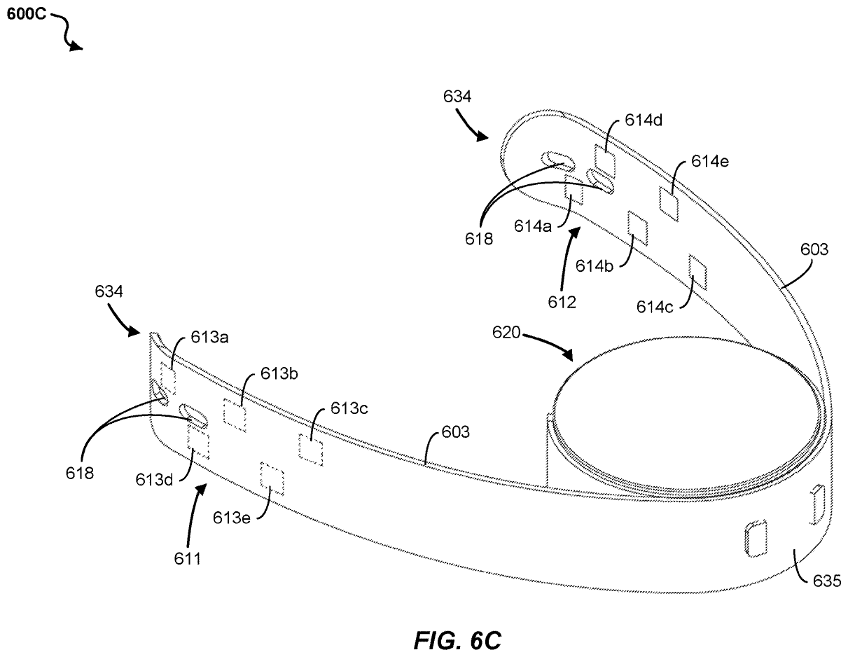

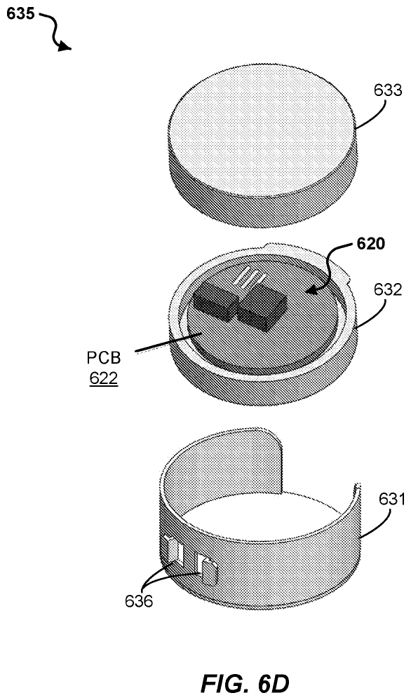

FIG. 6A shows a diagram depicting an example embodiment of the in vivo fluid flow sensor device 100X of FIG. 1B, shown in FIG. 6A as in vivo fluid flow sensor device 600A. The in vivo fluid flow sensor device 600A includes a sensor unit comprising at least one ultrasound sensor assembly 611 in electrical communication with an electronics unit 620 via electrical interconnections (not shown), in which the electrical interconnections are configured on a surface or within a material body of a clip band 603. The electronics unit 620 is housed in a connection apparatus 635 that connects the arms of the clip band 603. The ultrasound sensor assembly 611 includes a plurality of acoustic transducer elements 613, e.g., five acoustic transducer elements 613a, 613b, 613c, 613d, 613e in this example, positioned on the inner-facing side of the arm of the clip band 603. It is understood that this embodiment is not limited to five acoustic transducer elements for each ultrasound sensor assembly and can include less or more than the configuration shown in the diagram of FIG. 6A, e.g., including but not limited to one or more acoustic transducer element for the ultrasound sensor assembly 611. The other arm of the clip band 603 may include an embodiment of the reflector 516 (shown in FIG. 6A as reflector 616) configured to reflect acoustic signals transmitted by the ultrasound sensor assembly 611 that propagate across the anatomic structure, are affected by fluid flow through the anatomic structure, reflect from the reflector 616, and are received as returned acoustic signals by the ultrasound sensor assembly 611 (that correspond to the transmitted acoustic signals). In some embodiments, the reflector 616 may be attached to a backing or substrate 616s, which may attach to the inner surface of the arm of the clip band 603.

The clip band 603 is configured to secure the acoustic transducer elements 613 and the reflector 616 to position them in a fixed location with respect to each other. In some embodiments, for example the clip band 603 can be composed of a composite material having a durometer parameter of the polymer component (e.g., 20A to 95A), and stiffener material (e.g., made of a shape-set material, such Nitinol), which allows the clip band 603 to be pre-shaped and/or pre-trained (allowing the clip band 603 to be set within a first shape/configuration for delivery/implantation, after which it can take a second shape/configuration for deployment/operation). Some non-limiting examples of the composite material for the clip band 603 can include a Nitinol, gold, platinum, platinum/iridium, etc. encased in a polymer, such as silicone, polyethylene, polyimide, polyamide, or blended polyimide-polyamide, or other polymer material.

In some embodiments of the in vivo fluid flow sensor device 600A, the clip band 603 can include one or more openings 618 on at least one or both of the arms of the clip band 603 that allows for secondary attachment means of the device 600A to the target anatomic structure. For example, the openings 618 shown in the example of FIG. 6A include a portion of the clip band 603 between the two voids that are the openings 618, which provides an anchor site to apply a suture (e.g., stitch thread, wire, etc.) that affixes the arm of the clip band 603 to the anatomic structure (e.g., wall of the atrium of the heart).

FIG. 6B shows a diagram depicting an example embodiment of the in vivo fluid flow sensor device 600A of FIG. 6A, shown in FIG. 6B as in vivo fluid flow sensor device 600B. The in vivo fluid flow sensor device 600B includes a sensor unit comprising the ultrasound sensor assembly 611 and a second ultrasound sensor assembly 612, both in electrical communication with the electronics unit 620 via electrical interconnections (not shown), in which the electrical interconnections are configured on a surface or within a material body of the clip band 603. The electronics unit 620 is housed in a connection apparatus 635 that connects the arms of the clip band 603. The ultrasound sensor assembly 612 includes a plurality of acoustic transducer elements 614 positioned on the arm of the clip band 603 opposite to the arm on which the ultrasound sensor assembly 611 is disposed. In the example shown in FIG. 6B, the plurality of acoustic transducer elements includes five acoustic transducer elements 614a, 614b, 614c, 614d, 614e, which are positioned on the inner-facing side of the arm of the clip band 603 in a particular configuration with respect to each other and with respect to the acoustic transducer elements 613 of the ultrasound sensor assembly 611 on the opposing arm of the clip band 603. It is understood that this embodiment is not limited to five acoustic transducer elements for each ultrasound sensor assembly and can include less or more than the configuration shown in the diagram of FIG. 6B, e.g., including but not limited to one or more acoustic transducer element for the ultrasound sensor assembly 611. The clip band 603 is configured to secure the acoustic transducer elements 613 and the acoustic transducer elements 614 and position them in a fixed location with respect to each other. In some embodiments, the clip band 603 of the in vivo fluid flow sensor device 600B can include one or more openings 618 on at least one or both of the arms of the clip band 603 that allows for secondary attachment means of the device 600B to the target anatomic structure. For example, the openings 618 shown in the example of FIG. 6B include a portion of the clip band 603 between the two voids that are the openings 618, which provides an anchor site to apply a suture (e.g., stitch thread, wire, etc.) that affixes the arm of the clip band 603 to the anatomic structure (e.g., wall of the atrium of the heart).

FIG. 6C shows a diagram depicting an example embodiment of the in vivo fluid flow sensor device 600A of FIG. 6A and/or in vivo fluid flow sensor device 600B of FIG. 6B, shown in FIG. 6C as in vivo fluid flow sensor device 600C. The in vivo fluid flow sensor device 600C includes a sensor unit comprising the ultrasound sensor assembly 611 and/or the second ultrasound sensor assembly 612, both in electrical communication with the electronics unit 620 via electrical interconnections (not shown), in which the electrical interconnections are configured on a surface or within a material body of the clip band 603. The electronics unit 620 is housed in a connection apparatus 635 that connects the arms of the clip band 603. The in vivo fluid flow sensor device 600C includes an inner curved region 634 of the distal ends of each arm of the clip band 603, e.g., which can assist in facilitating attachment and conformation to the anatomic structure upon placement as well as aid in delivery of the device 600C, e.g., via a catheter The one or more openings 618 can be disposed on the inner curved region 634 and/or on the arm proximate the inner curved region 634 on at least one or both of the arms of the clip band 603, e.g., allowing for secondary attachment means of the device 600B to the target anatomic structure. In some embodiments, the connection apparatus 635 may be attached to the clip band 603 on the outside of the arms of the clip band 603 (not shown), which may be preferred for in vivo applications where the device 600C is deployed on a blood vessel (e.g., such as the vena cava or pulmonary artery or vein).

The example embodiments of FIGS. 6A-6C of the in vivo fluid flow sensor device 600A, 600B, and 600C, respectively, include a soft arm configuration of the clip band 603 for pliability and conformability to the intended site of placement. The arms of the clip band 603 can facilitate and maintain electronic transmissions through the interconnections (not shown) to the electronics unit 620 housed in the connection apparatus 635. The clip band 603 is able to enhance the contact of the device 600A, 600B, 600C with the anatomic structure for ultrasonic measurements to be detected. The arms of the clip band 603 can include a flexible, non-conductive, bioinert substrate material, upon or embedded within which is a flexible circuit and carrier for the ultrasound sensor assembly 611 and/or the second ultrasound sensor assembly 612. In this manner, the clip band 603 can undergo and withstand mechanical force during placement in an action to connect to the device to the intended position and during operation for long period of time (e.g., hundreds of millions of cycles, such as heart beats). In some embodiments, the arms of the linkage assembly 603 can use of a braid metallic or polymer sheets, which are subsequently interlaid with trace-lines for electrical connection.

FIG. 6D shows an exploded diagram depicting an example embodiment of the electronics unit 620 that is housed in the connection apparatus 635 of the in vivo fluid flow sensor device 600A, 600B, and 600C of FIGS. 6A-6C. In some embodiments, for example, the connection apparatus 635 includes a base housing 631 and a removably attachable cap 633 that can be secured to (e.g., hermetically seal) the base housing 631 to protect the interior components (e.g., the electronics unit 620) from the exterior environment (e.g., biofluids and/or bioconstituents). In some (optional) embodiments, for example, the connection apparatus includes an intermediate housing 632 to contain the electronics unit 620; for example, the intermediate housing 632 can be manufactured separately and in a multitude of physical conformations (e.g., shapes and sizes) to allow modularity with various embodiments of the connection apparatus 635 in any embodiment of the in vivo fluid flow sensor device 100X.

In some embodiments, the base housing 631, intermediate housing 632, and/or the cap 633 can be connected via a hermetic seal material (e.g., a non-permeable material, such as a metallic or metallic film (thin-film) and/or a woven cloth or polymer, such as parylene, a urethane, or a Teflon material) and coated to ensure a non-permeable interface between any of the base housing 631, intermediate housing 632, and/or the cap 633 to prevent fluids from breaching the connection apparatus 635 into the electronics unit 620, such as water or blood.

The electronics unit 620 includes a printed circuit board (PCB) 622 that facilitates a data processing unit, a wireless communications unit, and a power unit. For example, in some embodiments, the PCB 622 includes an embodiment of the data processing unit 121 to at least partially process the conditioned electrical signals to (i) produce data, e.g., in an analog or a digital form, and/or (ii) control functionality of the electronics unit 620 and/or the ultrasound sensor assembly 611 and/or 612. For example, the data processing unit 121 can be configured to manage data acquisition on data channels associated with the one or more acoustic transducers of the ultrasound sensor assembly 611 and/or 612. Also, for example, in some embodiments, the PCB 622 includes a power cell, e.g., which can be a super cap or small hermitic battery that is able to be recharged by inductance and/or function individually or in conjunction as a hybrid super capacitor battery, such that recharge and run times are balanced to be efficient for the required monitoring power and communication drain.

In some embodiments, the wireless communications unit of the electronics unit 620 includes an antenna, which can comprise Platinum, or 90/10 Platinum Iridium, Gold, Platinum pure wire, e.g., in a cross-sectional size from 0.000001 in² to 0.008 in², configured in a wire or tubular configuration. As an example, a shaped-set Nitinol wire with gold plating or a co-extruded gold external layer can be used. The exemplary wire(s) can be single or combined in a winded or braided configuration for optimal density to receive and transmit singles support the transfer of information in a single or multi directional pattern. In some embodiments, the antenna can be configured in any of the aforementioned patterns on a flat, flexible circuit geometry, such as a PCBA (printed circuit board assembly). In some embodiments, for example, the antenna can be configured as a snake antenna or a coil antenna fit into the electronic package; and/or in some embodiments, for example, the antenna can be configured as a power transfer antenna reference operable for battery charging.

In some embodiments, the wireless communications unit of the electronics unit 620 includes an integrated Bluetooth, galvanic or radio in an antenna communication system. In some implementations, the electronic signals transduced by the ultrasound sensor assembly 611 and/or 612 can be transmitted to a remote device outside of the patient's body as a communication data package, e.g., transmitted through the chest cavity to a receiving scanner, receiver transmitter, a receiving card (e.g., similar to an EKG port contact) or a base station equivalent.

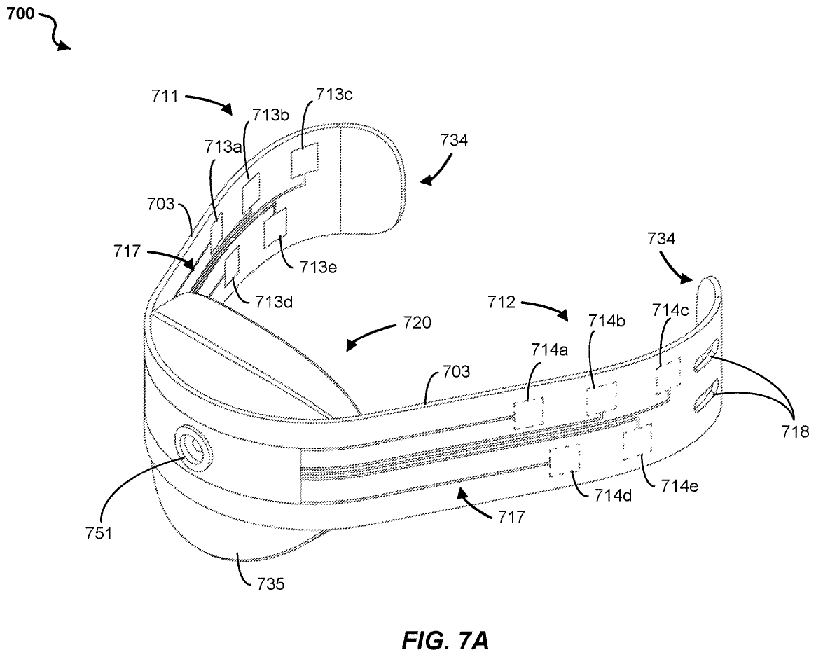

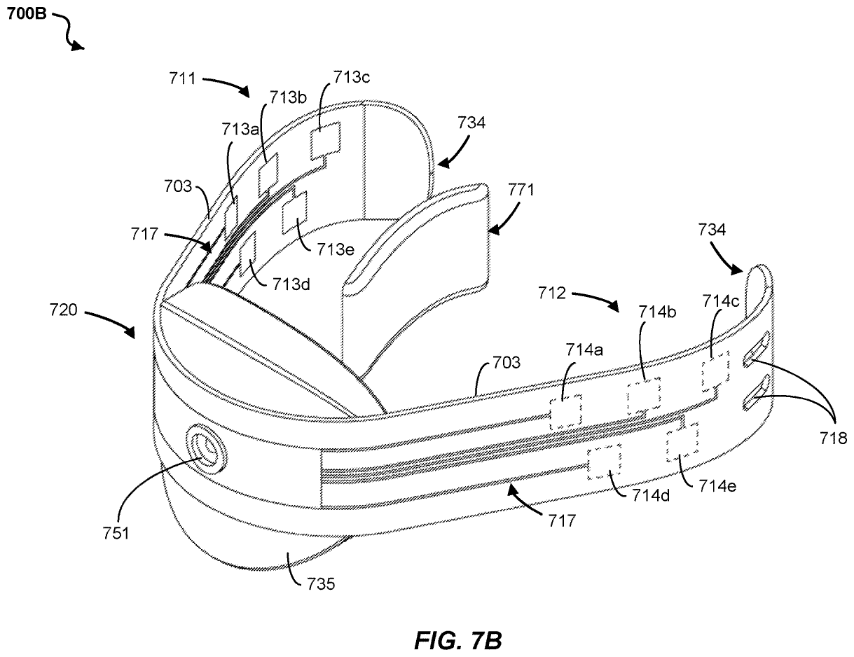

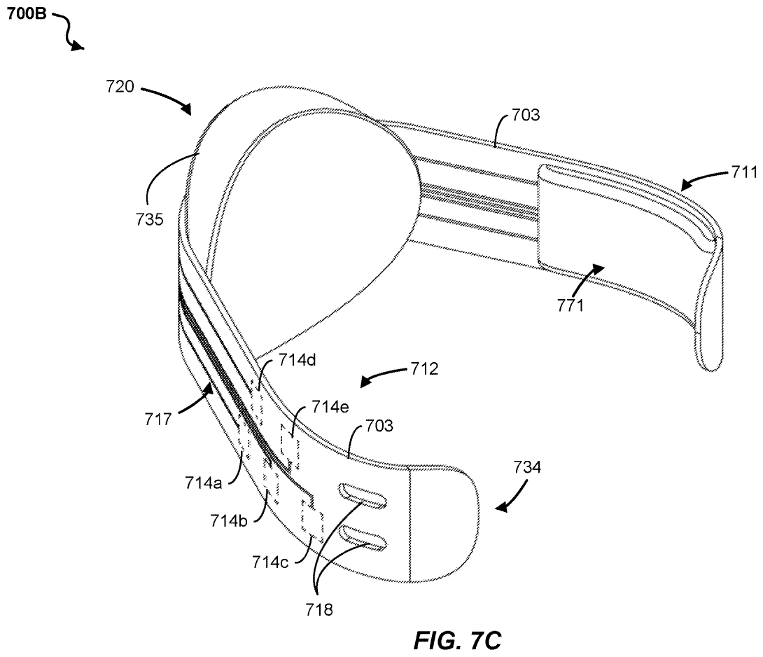

FIG. 7A shows a diagram depicting an example embodiment of the in vivo fluid flow sensor device 100X of FIG. 1B, shown in FIG. 7A as in vivo fluid flow sensor device 700. The in vivo fluid flow sensor device 700 includes a sensor unit comprising a first ultrasound sensor assembly 711 and a second ultrasound sensor assembly 612, both in electrical communication with the electronics unit 720 via electrical interconnections 717, in which the electrical interconnections 717 are configured on a surface or within a material body of an example embodiment of the clip band 603 (shown in FIG. 7A as clip band 703). The electronics unit 720 is housed in a connection apparatus 735 that connects the arms of the clip band 703.

The connection apparatus 735 can include features of the example embodiments of the connection apparatus 235, 235C, and/or 635, such as a spring, interconnection links, housing structures (e.g., base, cap, etc.), and other features described in relation to other connection apparatus embodiments disclosed herein. Referring to FIG. 7A, the connection apparatus 735 includes a power and/or data port 751 operable to interface (e.g., be in electrical communication and/or data communication) with the electronics unit 720 housed in the connection apparatus 735. For example, the electronics unit 720 of the in vivo fluid flow sensor device 700 can include a rechargeable power supply or in intermediary power supply that can be tethered to a separate power supply and/or data processing unit that is remote to the device 700, via the power and/or data port 751. In some example embodiments, the remote power supply and/or data processing unit may be an in vivo device located within the patient's body, but in a region that is easier to access to change a battery or wirelessly communicate with. For example, the remote in vivo device may be in wired communication and supply electrical power to the electronics unit 720 of the in vivo fluid flow sensor device 700 via a cable, wire, cord, etc. (e.g., that is coated by an insulative, bio-inert material) through the power and/or data port 751. As an illustrative example, the in vivo fluid flow sensor device 700 may be deployed in a confined space, such as inside or about the pericardium positioned at an atrium or ventricle, or positioned about the superior or inferior vena cava or a pulmonary artery, which is tethered to a larger in vivo power source device that is located in a cavity far away from the device 700 deployment site, e.g., such as one or a few or tens of centimeters away, such as a pleural cavity or cavity in the abdominal space, where there is more flexibility, size, and/or positioning to facilitate larger power supply and/or electronic components, and to allow for replacement of such components. In some examples, the remote power source may be located out of the body, with the power and/or data cable passing from power and/or data port 751 of the device 700 to the remote power source outside of the patient's body.

In some example implementations, an in vivo fluid flow sensor platform that includes the device 700 can include the optional one or more secondary sensor(s) 119 disposed within the remote in vivo device in wired communication with the sensor unit 720 via the cable, wire, cord, etc. that is connected through the power and/or data port 751. For instance, in some implementations, an example secondary sensor 119 can include an IMU to determine the patient user's motion (e.g., whether resting (e.g., laying down, sitting, etc.) or moving (e.g., walking, running, etc.). Furthermore, example secondary sensors(s) 119 as part of the in vivo fluid flow sensor platform can include, in some embodiments, an acoustic sensor to monitor breathing (respiratory parameters) by the patient user (e.g., respiratory rate of inhalation and exhalation, turbulent airflow, etc.) being monitored by the in vivo fluid flow sensor device 700. And yet further, example secondary sensors(s) 119 as part of the in vivo fluid flow sensor platform can include, in some embodiments, an EKG monitor (e.g., external wearable cardiac monitor or an in vivo insertable cardiac monitor) to measure electrocardiograms of the patient user being monitored by the in vivo fluid flow sensor device 700. In this manner, for example, the in vivo fluid flow sensor device 700 can measure blood flow through the target cardiac anatomy simultaneous with (temporally in sync with) sensing information of the patient user's motile state, breathing state, and/or heart rate.

In some example implementations, the remote in vivo device of the in vivo fluid flow sensor platform (that is in wired communication with the in vivo fluid flow sensor device 700) can include a secondary transmission device (e.g., wireless communication unit) for the sake of power control and optimal continuous data management, e.g., which can be communicated to the remote device 130 (e.g., base station 130B and/or mobile communication device 130A). For example, data transmissions by the example secondary transmission device (deployed remote from the device 700) can wirelessly transmit the data collected by the device 700 every minute, every second, etc. due to the reduced constraints and challenges on power consumption by the remote secondary transmission device with respect to the device 700. In some embodiments, for example, the secondary transmission device of the remote in vivo device can a transmitter, a receiver, and/or a transceiver with an antenna using a low power wireless communication protocol, e.g., such as Bluetooth Low Energy (BLE), Near Field Communication (NFC), low frequency radio frequency (RF) signal in a range of 3 kHz to 1.3 MHz, or other.

Referring to the example embodiment of the in vivo fluid flow sensor device 700 shown in FIG. 7A, the first ultrasound sensor assembly 711 includes a plurality of acoustic transducer elements 713 positioned on an arm of the clip band 703 that is opposite to the arm on which the second ultrasound sensor assembly 712 is disposed. In the example shown in FIG. 7A, the plurality of acoustic transducer elements 713 of the first ultrasound assembly 711 includes five acoustic transducer elements 713a, 713b, 713c, 713d, 713e, which are positioned on the inner-facing side of the arm of the clip band 703 in a particular configuration with respect to each other and with respect to the acoustic transducer elements 714 of the second ultrasound sensor assembly 712 on the opposing arm of the clip band 703. It is understood that this embodiment is not limited to five acoustic transducer elements for the first ultrasound sensor assembly 711 and can include less or more than the configuration shown in the diagram of FIG. 7A, e.g., including but not limited to one or more acoustic transducer element for the first ultrasound sensor assembly 711. The second ultrasound sensor assembly 712 includes a plurality of acoustic transducer elements 714 positioned on the arm of the clip band 703 opposite to the arm on which the first ultrasound sensor assembly 711 is disposed. In the example shown in FIG. 7A, the plurality of acoustic transducer elements includes five acoustic transducer elements 714a, 714b, 714c, 714d, 714e, which are positioned on the inner-facing side of the arm of the clip band 703 in a particular configuration with respect to each other and with respect to the acoustic transducer elements 713 of the first ultrasound sensor assembly 711 on the opposing arm of the clip band 703. It is understood that this embodiment is not limited to five acoustic transducer elements for each ultrasound sensor assembly and can include less or more than the configuration shown in the diagram of FIG. 7A, e.g., including but not limited to one or more acoustic transducer element for the second ultrasound sensor assembly 712.

The clip band 703 is configured to secure the acoustic transducer elements 713 and the acoustic transducer elements 714 and position them in a fixed location with respect to each other. In some embodiments, the clip band 703 of the in vivo fluid flow sensor device 700 can include one or more openings 718 on at least one or both of the arms of the clip band 703 that allows for secondary attachment means of the device 700B to the target anatomic structure. For example, the openings 718 shown in the example of FIG. 7A include a portion of the clip band 703 between the two voids that are the openings 718, which provides an anchor site to apply a suture (e.g., stitch thread, wire, etc.) that affixes the arm of the clip band 703 to the anatomic structure (e.g., wall of the atrium of the heart).

In some embodiments of the in vivo fluid flow sensor device 700, like the example shown in FIG. 7A, the device 700 includes an inner curved region 734 of the distal ends of each arm of the clip band 703, e.g., which can assist in facilitating attachment and conformation to the anatomic structure upon placement as well as aid in delivery of the device 700, e.g., via a catheter The one or more openings 718 can be disposed on the inner curved region 734 and/or on the arm proximate the inner curved region 734 on at least one or both of the arms of the clip band 703, e.g., allowing for secondary attachment means of the device 700 to the target anatomic structure.

FIGS. 7B and 7C show diagrams depicting an example embodiment of the in vivo fluid flow sensor device 100X of FIG. 1B, shown in FIG. 7B and in FIG. 7C as in vivo fluid flow sensor device 700B. The in vivo fluid flow sensor device 700B can be configured similar to the in vivo fluid flow sensor device 700 (shown previously in FIG. 7A), e.g., where the sensor unit comprises the ultrasound sensor assembly 711 and/or the ultrasound sensor assembly 712 in electrical communication with an electronics unit 720 via the electrical interconnections 717 (e.g., coupled to or partially housed within arms of the clip band 703), and where the connection apparatus 735 includes the power and/or data port 751 in electrical communication with the electronics unit 720 and a remote device. Yet, as shown in shown in FIG. 7B and in FIG. 7C, the in vivo fluid flow sensor device 700B includes an acoustic transducer pad 771 to protect the acoustic transducers 713 and/or 714 and to improve acoustic signal at transmission and reception (e.g., improve acoustic impedance matching) between the transducers and the biological tissue of the anatomic structure to which the device 700 is attached. FIG. 7B shows the in vivo fluid flow sensor device 700B in an exploded view where the acoustic transducer pad 771 is detached from the ultrasound sensor assembly 711, and FIG. 7C shows the in vivo fluid flow sensor device 700B in a rotated view (with respect to FIG. 7B) but where the acoustic transducer pad 771 is coupled to the arm of the clip band 703 and/or (at least one of) the plurality of acoustic transducer elements 713 of the ultrasound sensor assembly 711 positioned on the arm of the clip band 703. While not shown in FIGS. 7B and 7C, a second acoustic transducer pad 771 may be coupled to the opposing arm of the clip band 703 and/or (at least one of) the plurality of acoustic transducer elements 714 of the ultrasound sensor assembly 712.

In various embodiments, the acoustic transducer pad 771 can comprise hydrogel, which may be packaged in a gelatin form, or a fluid form, with a casing comprising a polymer material having a hardness of up to 40 A durometer (e.g., silicone, polyethylene, or other). In example implementations of the in vivo fluid flow sensor device 700 employing the acoustic transducer pad 771, the acoustic transducer pad 771 can further provide a contour and cushion against cardiac tissue, e.g., exterior tissue of the walls of the atrium or ventricle when the in vivo fluid flow sensor device 700 is deployed within the pericardium, as well as the epithelial tissue of the major blood vessel(s) such as the vena cava or pulmonary artery or vein. For example, the acoustic transducer pad 771 can account for contour variations with the anatomic structure the device 700 is directly interfaced with. Moreover, for example, the acoustic transducer pad 771 is operable to support signal isolation from noise and provide noise deflection for optimal sensing capability. The acoustic transducer pad 771 is able to reduce risk of damage from edge friability, e.g., by absorbing mechanical forces potentially incident upon the acoustic transducer elements during deployment and during operation (e.g., cycling motion of anatomic structure) that can lead to acute harm or long-term wear and tear, e.g., notably while not impacting the acoustic signal transfer between the anatomic structure and the acoustic transducer elements. Similarly, the acoustic transducer pad 771 is able to reduce the risk of tissue abrasion to the anatomic structure to preserve contact viability between the device and the biological tissue.

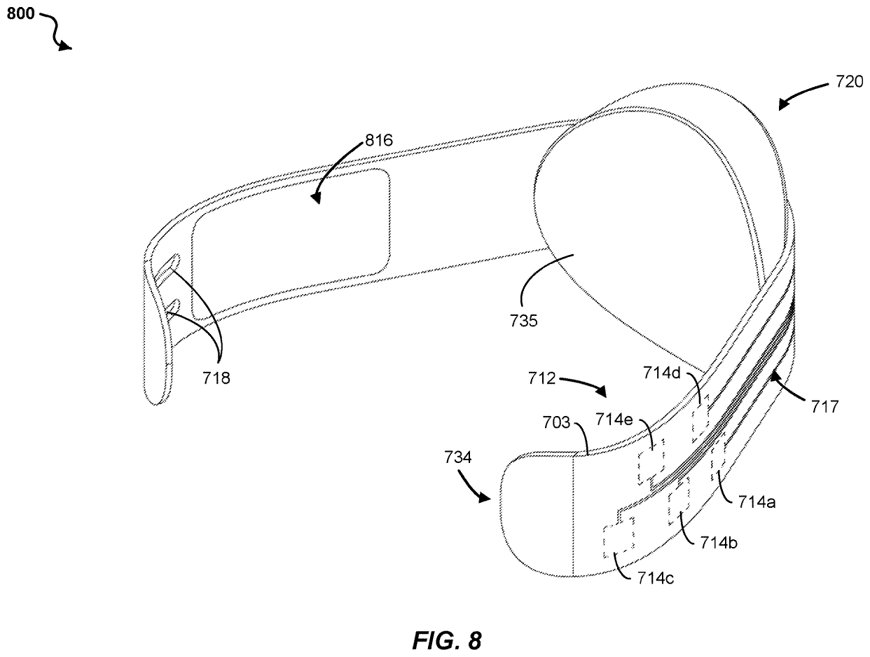

FIG. 8 shows a diagram depicting an example embodiment of the in vivo fluid flow sensor device 100X of FIG. 1B, shown in FIG. 8 as in vivo fluid flow sensor device 800. The in vivo fluid flow sensor device 800 can be configured similar to the in vivo fluid flow sensor device 700 (shown previously in FIG. 7A) and/or in vivo fluid flow sensor device 700B (shown previously in FIG. 7B and FIG. 7C, having at least one acoustic transducer pad 771), e.g., where the sensor unit comprises an ultrasound sensor assembly (shown in FIG. 8 as the ultrasound sensor assembly 712) in electrical communication with an electronics unit 720 via the electrical interconnections 717 (e.g., coupled to or partially housed within arms of the clip band 703), and where the connection apparatus 735 includes the power and/or data port 751 in electrical communication with the electronics unit 720 and a remote device. Yet, as shown in shown in FIG. 8, the in vivo fluid flow sensor device 800 includes an embodiment of the reflector 516 (shown in FIG. 8 as reflector 816) configured to reflect acoustic signals transmitted by the ultrasound sensor assembly 712 that propagate across the anatomic structure, are affected by fluid flow through the anatomic structure, reflect from the reflector 816, and are received as returned acoustic signals by the ultrasound sensor assembly 712 (that correspond to the transmitted acoustic signals). In some embodiments, the reflector 816 may be attached to a backing or substrate (not shown), which may attach to the inner surface of the opposing arm of the clip band 703 to the arm that facilitates the ultrasound sensor assembly 712.

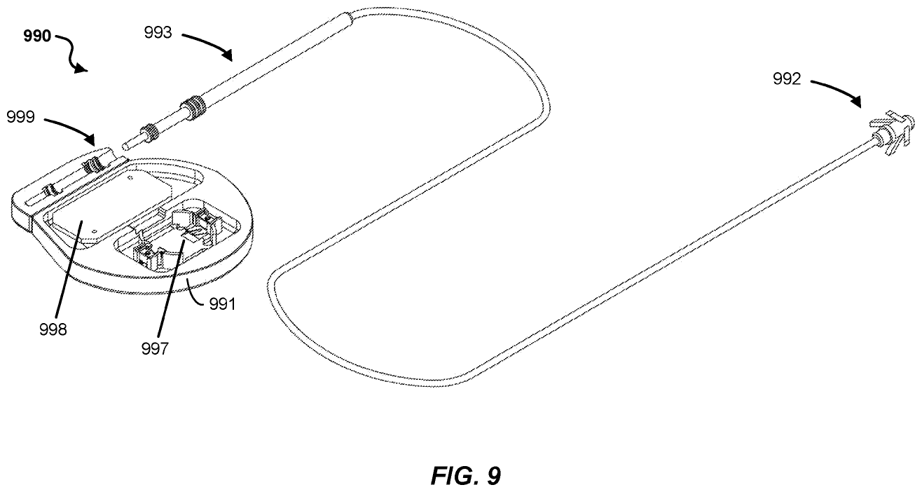

FIG. 9 shows a diagram of an example embodiment of a remote in vivo device 990 operable to be in wired communication with any of the example embodiments of the in vivo fluid flow sensor device 100X of FIG. 1B, such as the in vivo fluid flow sensor device 700, 700B, and/or 800 shown in FIG. 7A, FIGS. 7B-7C, and/or FIG. 8, respectively, or other embodiments disclosed herein. The remote in vivo device 990 includes a casing or housing 991, within which a power supply 998 and data processing and/or wireless communication unit 997 is housed.

Examples of the power supply 998 can include a battery, a fuel cell, or other electrical power source. In some embodiments, for example, the power supply 998 can be configured to be a replaceable battery and/or a rechargeable battery. For instance, in example implementations where the remote in vivo device 990 is located within the patient's body in an area or region that is relatively easy and safe for a clinician to access, e.g., such as the pleural cavity or a space in the abdominal area, the power supply 998 can be recharged. As such, the electrical energy stored in the power supply 998 can be used to supply the power supply in the electronics unit 120X of the example embodiment of the in vivo fluid flow sensor device 100X, which may have a relatively lower charge storage capacity than the power supply 998. The transfer of the electrical energy can be provided from the remote in vivo device 990 to the example in vivo fluid flow sensor device (e.g., in vivo fluid flow sensor device 700, 700B, 800 or other) via a cable, wire, or cord 993 that interfaces with the power and/or data port 751 of the example in vivo fluid flow sensor device 700, 700B, 800 via an interfacing terminus 992. In some embodiments of the remote in vivo device 990, for example, the cable, wire, or cord 993 can be detachable from the housing or casing 991 of the in vivo device 990 at a receiving port 999. In the example illustration of FIG. 9, the interfacing terminus 992 of the cable, wire, or cord 993 is shown to have at least one protruding structure, which can be used to create a locking system with the power and/or data port 751 to ensure securement (e.g., to avoid unwanted detachment of the cable, wire, or cord 993 in the patient's body from the example in vivo fluid flow sensor device 700, 700B, 800).

Examples of the data processing and/or wireless communication unit 997 can include a transmitter, a receiver, and/or a transceiver with an antenna using a low power wireless communication protocol, e.g., such as Bluetooth Low Energy (BLE), Near Field Communication (NFC), low frequency radio frequency (RF) signal, in data communication with one or more processors coupled to a memory. In this manner, the data processing unit of the electronics unit 720 may manage data processing functions using less on-board power and offload more processing-intensive data processing tasks to the one or more processors and memory of the data processing and/or wireless communication unit 997. Moreover, the data processing and/or wireless communication unit 997 may serve to at least partially control certain device functions of the example in vivo fluid flow sensor device 700, 700B, 800.

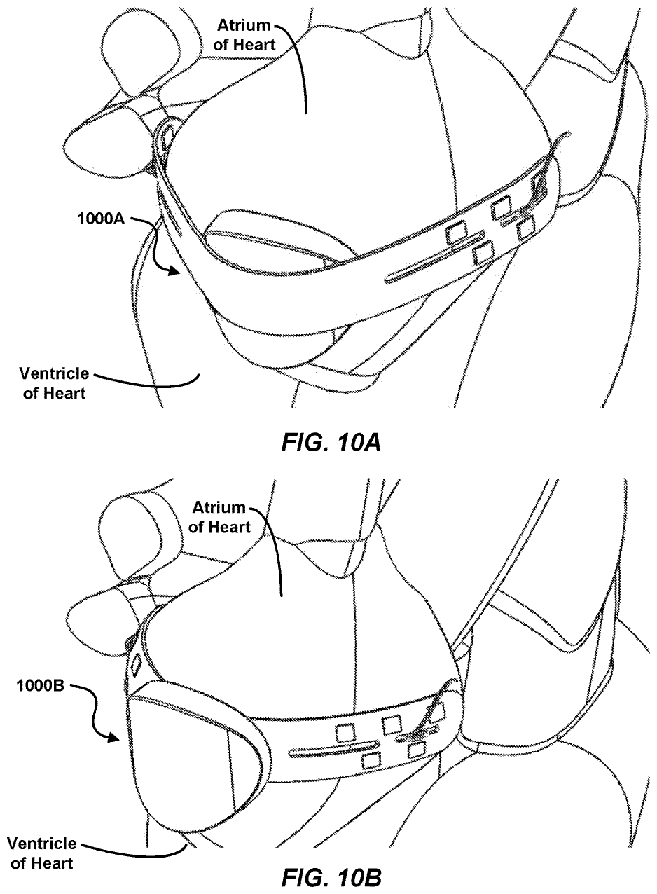

FIGS. 10A and 10B show diagrams illustrating an example embodiment of the in vivo fluid flow sensor device 100X of FIG. 1B, shown in FIGS. 10A and 10B as in vivo fluid flow sensor device 1000A and 1000B, respectively, where the in vivo fluid flow sensor device 1000A, 1000B is attached to a heart of a patient user in an example implementation of the device. In this example, the in vivo fluid flow sensor device 1000A, 1000B is configured to assess blood flow across the mitral valve, e.g., for assessing MR, by placing the ultrasound sensor assemblies on opposing sides of the external surface of the atrium of the heart about the mitral valve. As illustrated in FIGS. 10A and 10B, the arms of the in vivo fluid flow sensor device 1000A and 1000B (e.g., arms of an example embodiment of the linkage assembly 103 of the in vivo fluid flow sensor device 100X as the clip band 603 and/or 703) are capable of extending about an organ, such as the atrium of the heart, with sufficient pliability that can support various orientations for optimizing functional alignment with the target anatomic structure.

Further, the optimal functional alignment can be preserved by the arms of the linkage assembly (e.g., degree of flex), which can position and secure the in vivo fluid flow sensor device 1000A, 1000B to the target anatomic structure individually or in compilation with (optional) one or more secondary attachment mechanisms, e.g., such as sutures (secured at suture ports), adhesives, or anchor structures (secured at anchoring ports), which can optimize the placement against the intended area of the organ/tissue for consistent measurements (e.g., initial monitoring to establish calibration, then comparison by constant monitoring and comparison from the baseline). Also, in some implementations, for example, multiple in vivo fluid flow sensor devices 1000A, 1000B can be deployed at different locations of the same anatomic structure.

The example embodiment of the in vivo fluid flow sensor device 1000A includes the connection apparatus disposed on the inside of the clip band linkage assembly (e.g., proximate to the anatomic structure the device 1000A is attached). Yet, the example embodiment of the in vivo fluid flow sensor device 1000B includes the connection apparatus disposed on the outside of the clip band linkage assembly (e.g., distal from the anatomic structure the device 1000B is attached). It is understood that the configuration of the connection apparatus can be on the inside or the outside of the linkage assembly for various embodiments of the in vivo fluid flow sensor device 100X, including any embodiment of the linkage assembly 103.

### Displacement-Mediated In Vivo Acoustic Sensor Device

In some embodiments of the in vivo acoustic sensor device 100Y, for example, the acoustic sensor 111Y includes a displacement-mediated acoustic sensor, which can include an internal microphone-based acoustic sensor, referred to herein as a microphone acoustic sensor, or simply a microphone.

FIG. 11A shows a diagram depicting an exemplary acoustic sensor in a portion of an example embodiment of the implantable medical device 100 of the present technology, shown as IMD 1100A in FIG. 11A. As shown in FIG. 11A, the IMD 1100A includes a housing 1102 and a membrane 1104, and together the housing 1102 and membrane 1104 create a hermetic enclosure for an example embodiment of the in vivo acoustic sensor 100Y. In some embodiments, for example, the membrane 1104 is a deflective membrane, e.g., capable of elastic deformation to change conformations between a relaxed state and a flexed state (e.g., deflated and/or inflated). In such embodiments, the acoustic sensor can be configured to operate as a microphone, i.e., an instrument that converts sound (mechanical waves) into electrical signals, which can be signal processed (e.g., amplified). In some embodiments, for example, the membrane 1104 includes titanium (Ti) or other strong, impermeable, biocompatible and relatively inflexible metal, such as a surgical-grade stainless steel (e.g., MP35N) or cobalt chromium alloys or Nitinol, or a high-purity aluminum oxide that is strong, impermeable, biocompatible and flexible to transduce pressure differentials. In some embodiments, the housing 1102 and the membrane 1104 are formed from the same material, however the membrane 1104 has properties, such as thickness or elasticity, that allows it to achieve a desired response to pressure variation while maintaining hermeticity. In some embodiments, the housing 1102 and the membrane 1104 are made from different materials but are securely joined together so as to create a hermetic enclosure. In some embodiments, for example, the membrane 1104 can be configured in a circular shape, such that acoustic signals (e.g., mechanical waves) emanated from an in vivo anatomic structure, such as a portion of the heart or the lungs, that are received at the membrane are detectable by the relationship between the pressure differential (q) and the flexural rigidity (D) of the membrane 1104, in accordance with Equation (1):

$${∇}^{2} {∇}^{2} w = - \frac{q}{D} \qquad ( 1 )$$

where ω=ω(r) for exemplary embodiments where the membrane is configured as a circular plate (i.e., a cylinder with coordinates: radius r, angle θ, and height z), such that:

$${∇}^{2} w ≡ \frac{1}{r} \frac{\partial }{\partial r} ( r \frac{\partial }{\partial r} ) + \frac{1}{{r}^{2}} \frac{{\partial }^{2} w}{\partial {θ}^{2}} + \frac{{\partial }^{2} w}{\partial {𝓏}^{2}} \qquad ( 2 )$$

and where D is a function of the total thickness (H), i.e., H=2 h, the elastic modulus (E), and Poisson's ratio (v) of the material of the membrane.

$$D := \frac{2 {h}^{3} E}{3 ( 1 - {v}^{2} )} = \frac{{H}^{3} E}{12 ( 1 - {v}^{2} )} \qquad ( 3 )$$

Also, as shown in the diagram of FIG. 11A, 1106 indicates an external medium outside of the housing 1102, such as a gas, liquid, or solid, including tissue within a host, that is capable of transmitting variations in pressure. The external medium 1106 is in direct contact with an outside surface of the hermetic enclosure, and in particular is in contact with the outside surface of the membrane 1104. Also, as shown in FIG. 11A, 1108 indicates an internal medium of the housing 1102, such as a gas, liquid, gel or other medium inside the hermetic enclosure of the example embodiment of the in vivo acoustic sensor 100Y for the example IMD 1100A, in which the internal medium 1108 is capable of transmitting variations in pressure such as an acoustic signal. The internal medium 1108 is in direct contact with an inside surface of the hermetic enclosure from housing 1102 and membrane 1104, and in particular is in contact with the inside surface of the membrane 1104.

FIG. 11B shows a cross-sectional view showing an enlarged view of a portion 1107 of an example embodiment of the in vivo acoustic sensor 100Y for the example IMD 1100A, now identified as portion 1107B in FIG. 11B, with the membrane of the acoustic sensor in a relaxed state. In FIG. 11B, as shown in cross-section, the housing 1102 is present and shown on each end of the membrane 1104. As in FIG. 11B, the external medium 1106 is present outside the housing 1102 and in contact with the exterior surface of the membrane 1104, and a separate internal medium 1108 is present inside the housing 1102 and in contact with the interior surface of the membrane 1104. In FIG. 1B, the membrane 1104 is shown to travel in a substantially straight line between the portions of the housing 1102 to either side of the membrane 1104, which indicates that the membrane 1104 is in a relaxed state due to equilibrium or balance of the pressures in media 1106 and 1108.

FIG. 11C shows a cross-sectional view showing an enlarged portion of the image of FIG. 11A, in analogy to FIG. 11B, now identified as portion 1107E in FIG. 11C, but with the membrane 1104 of the acoustic sensor in a deflected state. FIG. 11C shows the effect of a pressure differential between the inner surface of the hermetic enclosure and the outer surface of the hermetic enclosure. In particular, the outside pressure (Po) in the external medium 1106 acting on the IMD is greater than the inside pressure (Pi) in the internal medium 108 of the IMD. Accordingly, the membrane 1104 is deflected into the hermetic enclosure occupied by internal medium 1108, by an amount that can be defined by the distance between point 1112a, where the membrane 1104 is in a relaxed state, and point 1112b, where the membrane 1104 is in a deflected state. For example, a pressure differential across a plate, e.g., such as the membrane 1104, acts as uniform load and will deform the plate, resulting in stress distribution(s) proportional to the pressure differential.

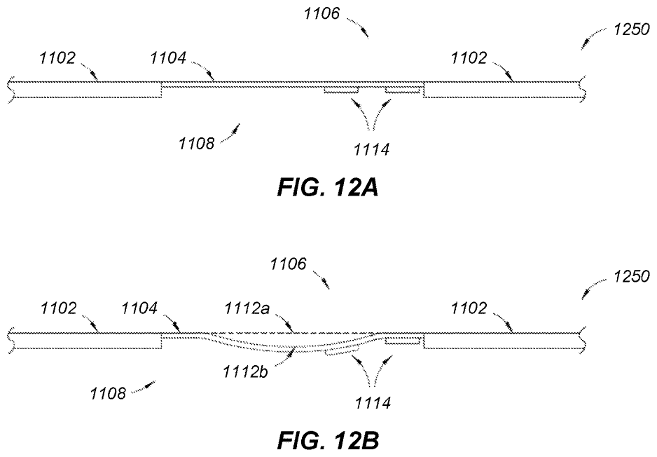

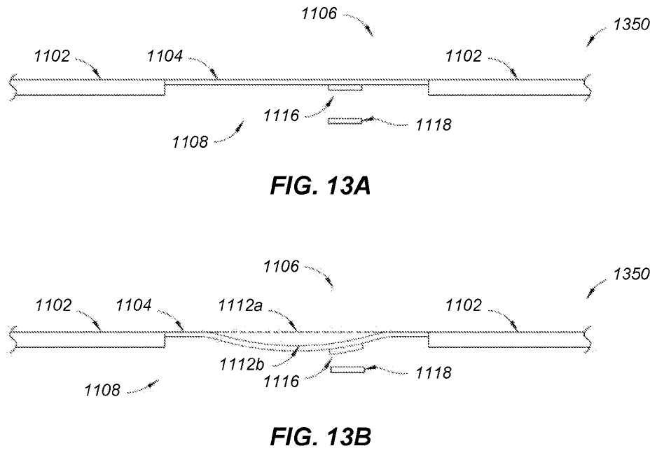

Once a deflectable membrane of the acoustic sensor (e.g., a microphone) is utilized for converting a pressure differential into deflection, as shown in FIG. 11C, there are multiple ways to convert that deflection into an electrical signal that can be measured in terms of presence of deflection and/or extent of deflection, e.g., optionally as a function of time. Exemplary ways to convert that deflection into an electrical signal are shown by example embodiments of the displacement-mediated in vivo acoustic sensor in FIGS. 12A, 12B, 13A and 13B.

FIGS. 12A and 12B show a diagram depicting an exemplary displacement-mediated acoustic sensor configured as an in vivo microphone for some example embodiments of the in vivo acoustic sensor 100Y for the IMD 100, in accordance with the present technology, in which the exemplary acoustic sensor includes a strain gauge and/or a piezoelectric element. FIG. 12A shows a cross-sectional view showing an exemplary acoustic sensor (e.g., microphone) in a portion of an exemplary IMD 1250 of the present technology, including a sensor 1114, which can include one or both of a strain gauge and a piezoelectric element, that is configured with the membrane 1104 of the exemplary acoustic sensor (e.g., microphone) in a relaxed state. FIG. 12B shows a cross-sectional view diagram showing the exemplary acoustic sensor (e.g., microphone) in the portion of the exemplary IMD 1250, including the sensor 1114 (that includes one or both of a strain gauge and a piezoelectric element), in analogy with FIG. 12A, but with the membrane 1104 of the exemplary acoustic sensor (e.g., microphone) in a deflected state.

The diagrams of FIGS. 12A and 12B illustrate how the example embodiments of the sensor 1114, i.e., one or more strain gauges and/or one or more piezoelectric elements, may be mounted on the membrane 1104 such that deflection of the membrane 1104 imparts strain on the strain gauge(s) 1114 and/or piezoelectric element(s) 1114. A pressure differential between inner (‘Pi’) and outer surfaces (‘Po’) of the hermetic enclosure of IMD 1250 wherein the membrane 1104 is deflected by an amount identified as the distance between points 1112a and 1112b in response to a difference in pressure between inner and outer surfaces of the hermetic enclosure, resulting in bending moment (strain) in the example strain gauge(s) 1114 and/or piezoelectric element(s) 1114. Thus, the sensor 114 may be used to detect and measure the extent of deflection, and accordingly the difference in pressure between the inside and outside of the membrane 1104.

In example implementations of the sensor 1114 including a strain gauge for the exemplary IMD 1250, for example, the resistance of the strain gauge 1114 changes in response to the strain imparted by deflecting membrane 1104. This resistance may be converted proportionally to electrical signals using well known techniques, e.g., a Wheatstone bridge. Alternatively, detection of static pressure changes (e.g., atmospheric pressure, blood pressure, etc.) may also be accomplished by making use of strain gauges and known technology. In some embodiments, the present technology makes use of a strain gauge as a component of a microphone to form an acoustic sensor, and in particular an acoustic sensor as a component of an IMD.

In example implementations of the sensor 1114 including a piezoelectric element for the exemplary IMD 1250, for example, the piezoelectric element(s) 114 are mounted on the internal surface of the membrane 1104, such that the piezoelectric element(s) generates an electrical charge in response to the strain imparted by deflecting membrane 1104. This electrical charge may be converted proportionally to electrical signals using well known techniques, e.g., a charge amplifier. In some embodiments, the present technology makes use of a piezoelectric element as a component of a microphone to form an acoustic sensor, and in particular an acoustic sensor as a component of an IMD.

FIGS. 13A and 13B show a diagram depicting an exemplary acoustic sensor configured as an in vivo microphone for some example embodiments of the in vivo acoustic sensor 100Y for the IMD 100, in accordance with the present technology, including a capacitive electrode sensor or a sensor with electret condenser elements. FIG. 13A shows a cross-sectional view showing an exemplary acoustic sensor (e.g., membrane 1104) in a portion of an exemplary IMD 1350 of the present technology, including a sensor 1116, which can include one or both of a capacitive electrode sensor and/or a sensor with electret condenser elements, with the membrane 1104 of the microphone in a relaxed state. FIG. 13B shows a cross-sectional view showing the exemplary acoustic sensor (e.g., microphone 1104) in the portion of the exemplary IMD 1350, including the sensor 116 (that includes one or both of the capacitive electrode sensor and/or the sensor with electret condenser elements), in analogy with FIG. 13A, but with the membrane 1104 of the microphone in an extended or deflected state.

In some example embodiments of the exemplary acoustic sensor (e.g., microphone) of FIG. 13A or 13B, either capacitive electrode 1116 or electret condenser elements 1116 are mounted on the membrane 1104 such that deflection of the membrane 1104 changes the separation between sensor 1116 (e.g., either a capacitive electrode or electret condenser elements) and fixed electrode 1118. A pressure differential between inner (‘Pi’) and outer surfaces (‘Po’) of the hermetic enclosure wherein the membrane 1104 is deflected by an amount described by the distance of points 1112a and 1112b in response to a difference in pressure between inner and outer surfaces of the hermetic enclosure. The deflection of membrane 1104 also changes the spacing between the sensor 1116 (either the capacitive electrode or electret condenser elements) and fixed electrode 1118, causing a change of the electrical charge on the fixed electrode 1118. The resulting change in charge on fixed electrode 118 is detected and amplified by either a charge or voltage amplifier.

In example implementations of the electrode 1116 including the electret condenser element for the exemplary IMD 1350, for example, an electret condenser element (at 1116) is mounted on the internal surface of the membrane 1104. An electret condenser element 116 on a deflected surface (such as the interior surface of the membrane 1104) is capable of generating a charge signal on a nearby electrode 1118. This charge signal may be converted proportionally to electrical signals using well known techniques, e.g., using charge or voltage amplifiers. In some embodiments, the present technology makes use of an electret condenser element as a component of a microphone to form an acoustic sensor, and in particular an acoustic sensor as a component of an IMD.

In example implementations of the electrode 1116 including the capacitive element for the exemplary IMD 1350, for example a simple capacitive electrode (at 1116) is mounted on the internal surface of the membrane 1104. Application of a constant voltage bias on a conductive moving surface (such as the interior surface of the membrane 1104) is capable of generating a charge signal on a nearby electrode 1118 that can be extracted as an electrical signal. This charge signal may be converted proportionally to electrical signals using well known techniques, e.g., using charge or voltage amplifiers. In some embodiments, the present technology makes use of a capacitive electrode as a component of a microphone to form an acoustic sensor, and in particular an acoustic sensor as a component of an IMD.

For example, an exemplary IMD with a microphone-type displacement-mediated acoustic sensor can be suitable for use for at least a relatively short period of time (e.g., several months up to one year). Over time, biofilm may deposit on the diaphragm and thereby change the responsiveness of the diaphragm to sound waves over time. However, in some embodiments of the IMD with a microphone as the displacement-mediated acoustic sensor, the IMD can include preventative or mitigating components against such biofouling including, but not limited to, protective and non-impeding membranes, coatings (e.g., chemical or biological) or elution mechanisms, such that the IMD is able to manage an environment about its location with minimal to no biological reaction to enable both short and long periods of functional time, and where such environments can include intestinal fluid, muscle regrowth, scar tissue, or capillary intramuscular growth (e.g., by including a microporous membrane with the IMD, a microporous membrane with a elution coating for anticoagulation for capillary in-growth, and/or a non-porous monomer in-itself or carrying a drug elution compound). Yet, for some embodiments of the IMD for long term acoustic detection and measurement, a displacement-mediated acoustic sensor that does not have a sensing element that directly contacts tissue in the host may be utilized in an IMD of the present technology. One such acoustic sensor is an accelerometer, for example, which is discussed next, which can be encased in a housing and thus protected from coming into direct contact with the host tissue and host fluids with reduced sensitivity to biofilm deposits.

In some embodiments of a displacement-mediated acoustic sensor for the in vivo acoustic sensor 100Y, for example, the acoustic sensor 111Y includes an accelerometer, which may be referred to herein as an acoustic accelerometer. In some embodiments, the acoustic accelerometer is capable of high fidelity (HF) acceleration sensing. For example, when performing high fidelity acceleration sensing, the acoustic accelerometer can be configured to measure mechanical waves within the body and resolve accelerations as small as 100 micro-g, or 10 micro-g, or 1 micro-g. In some embodiments, the acoustic accelerometer senses acceleration frequencies up to at least 5000 Hz, or up to at least 4000 Hz, or up to at least 3000 Hz, or up to at least 2000 Hz, or up to at least 1000 Hz, or up to about 900 Hz, or up to about 800 Hz, or up to about 700 Hz, or up to about 600 Hz, or up to about 500 Hz.

The acoustic accelerometer may sense the acceleration frequencies of interest because the accelerometer may be designed to be limited to the frequencies of interest, i.e., it is only capable of sensing in the frequency range of interest for an acoustic accelerometer. Alternatively, or additionally, the accelerometer is capable of sensing at the frequencies of interest for an acoustic accelerometer as well as frequencies that are not of interest for an acoustic accelerometer, however in this case the IMD may contain firmware that limits the accelerometer to sensing at the frequencies of interest and not at the frequencies that are not of interest for performance as an acoustic accelerometer. In this way, for example, the accelerometer does not consume power and other IMD resources due to sensing at frequencies that are not relevant to monitoring acoustic signals.

In some embodiments, the acoustic accelerometer operates at a high frequency sampled data rate. When operating at a high frequency sampled data rate, the accelerometer samples acceleration data up to 20,000 samples per second, or up to 18,000 samples per second, or up to 16,000 samples per second, or up to 14,000 samples per second, or up to 12,000 samples per second, or up to 10,000 samples per second, or up to 8,000 samples per second, or up to 6,000 samples per second, or up to 4,000 samples per second, or up to 2,000 samples per second, or up to 1,800 samples per second, or up to 1,6000 samples per second, or up to 1,400 samples per second, or up to 1,200 samples per second, or up to 1,000 samples per second, or up to 800 samples per second. In some embodiments, the accelerometer operates at a data sampling rate of between 1,400 to 14,000 samples per second. In some embodiments, the displacement-mediated acoustic sensor is configured such that the accelerometer operates at a high frequency sampled data rate.

In some embodiments, the acoustic accelerometer of the displacement-mediated acoustic sensor operates at a low noise. When operating at a low noise level, the accelerometer input-referred noise level, referred to as spectral noise density, of less than 500 micro-g/rtHz, or less than 400 micro-g/rtHz, or less than 300 micro-g/rtHz, or less than 200 micro-g/rtHz, or less than 100 micro-g/rtHz, or less than 75 micro-g/rtHz, or less than 50 micro-g/rtHz, or less than 25 micro-g/rtHz, or less than 10 micro-g/rtHz, or less than 5 micro-g/rtHz, or less than 1 micro-g/rtHz, or less than 0.8 micro-g/rtHz, or less than 0.5 micro-g/rtHz, or less than 0.1 micro-g/rtHz. In some embodiments, the accelerometer operates at an input-referred noise level of between 200 micro-g/rtHz and 0.5 micro-g/rtHz.

In some embodiments, the acoustic accelerometer of the displacement-mediated acoustic sensor operates at a high precision. When operating at a high precision, the accelerometer has a precision of less than 50 micro-g, or less than 40 micro-g, or less than 30 micro-g, or less than 20 micro-g, or less than 10 micro-g, or less than 5 micro-g, or less than 1 micro-g, or less than 0.5 micro-g, or less than 0.1 micro-g, or less than 0.05 micro-g, or less than 0.01 micro-g. In some embodiments, the accelerometer operates at a precision of between 10 micro-g and 1 micro-g.

In some embodiments, the acoustic accelerometer is a single axis accelerometer. In some embodiments, the acoustic accelerometer is a multi-axis accelerometer. In some embodiments, the multi axis accelerometer is a dual axis accelerometer. In some embodiments, the multi-axis accelerometer is a tri-axis accelerometer. A multi-axis (dual axis or triaxial) is preferred for multidirectional acoustic detection making sensing efficacy less dependent on placement within the host's anatomy or at least adaptive to variations in anatomic placement.

In some embodiments, the acoustic accelerometer operates at 16 to 24 bits. In some embodiments, the acoustic accelerometer operates at 0.01 to 0.1 milli-g resolution.

In some embodiments, the acoustic accelerometer is rigidly mounted to the housing 101Y of the displacement-mediated acoustic sensor. In some embodiments, the housing is rigid, e.g., it does not compress when contacted by acoustic signals. Rather than compressing, the housing moves within the host in response to contacting the acoustic signals, and because the acoustic accelerometer is rigidly mounted, either directly or indirectly to the inside of the housing, the acoustic accelerometer undergoes the same movement as does the housing upon the housing being impacted by acoustic signals.

As mentioned elsewhere herein, acoustic waves (or sound) are pressure waves that propagate through various materials including gas, fluid, or solids. As a result of the pressure waves, sound in air produces a vibratory motion of the air molecules such that the velocity of the air molecules is proportional to the pressure wave amplitude. If the molecule velocity is ‘v’, then v=p/Z where ‘p’ is the sound pressure, and ‘Z’ is the acoustic impedance for the material the sound is propagating through. The acoustic impedance ‘Z’ is different for different materials. This relationship between acoustic pressure waves and particle/molecule velocity is the basis for sensing acoustic pressure as particle acceleration. For pressure waves at a frequency ‘f’ the molecules are vibrating at this frequency with velocity v=p/Z as noted above, and the molecules' acceleration ‘a’ is a=2*pi*f*v=2*pi*f*p/Z. Thus, the molecule acceleration is proportional to the sound intensity or pressure wave amplitude (p) and the frequency (f).

When an example IMD having a suitable accelerometer according to the present technology is placed in the material through which acoustic pressure waves are propagating, then under certain conditions the accelerometer will see the same acceleration that the surrounding tissue molecules see or experience. In some embodiments, the conditions for the accelerometer to “move with” the tissue in response to propagating acoustic pressure waves include: (i) the IMD mass density is similar to or lighter than the surrounding material, and (ii) the physical dimension (size) of the accelerometer in the direction of acoustic pressure wave propagation is similar to or smaller than the wavelength of the pressure waves. Thus, in addition to consideration of accelerometer performance requirements as described above (i.e., frequency range sensed, sampled date rate, noise, precision) the physical behavior of acoustic waves propagating in a medium may also impose dimensional and mass density limitations on an example displacement-mediated acoustic sensor for an IMD containing an acoustical accelerometer.

### Example Auxiliary Sensors for In Vivo Acoustic Sensor Device

In addition to the one or more acoustic sensors, the IMD 100 may optionally include one or more auxiliary sensors. An auxiliary sensor can detect and optionally measure a non-acoustic signal or feature of the host. In some embodiments, the auxiliary sensor provides data that is complementary to the data obtained by the acoustic sensor. In other words, the data obtained from the auxiliary sensor is evaluated in combination with an evaluation of the data obtained from the acoustic sensor, to provide deeper insight into the physical state of the host and the conditions under which acoustic data is collected. In some embodiments, the acoustic sensor and the auxiliary sensor operate simultaneously, so that data is obtained by the acoustic sensor at the same time, i.e., during an overlapping time period, that data is obtained by the auxiliary sensor. For example, during a single 10 second period, both the acoustic sensor and the auxiliary sensor are obtaining data. In another example, the data sensed by the auxiliary sensor prompts the IMD to activate the acoustic sensor, whereupon the acoustic sensor obtains measurements, optionally while the auxiliary sensor continues to obtain data.

In some embodiments, for example, the in vivo acoustic sensor device 100Y optionally includes a sensor to measure an electrocardiogram (ECG or EKG) of the host, for example, to obtain data that can be evaluated to provide a galvanic-based EKG of the host. The exemplary ECG sensor to allow the creation of an EKG may look at the conductivity of the heart. For example, the in vivo acoustic sensor device 100Y may include two electrodes on either end of the in vivo acoustic sensor device 100Y, where the ends of those two electrodes are separated by a space that creates an electrical potential, which creates spikes as the heart is beating, where those spikes give rise to the EKG.

Considering the heart performance of the host, that heart has a heart rate, the rate at which the heart beats, which is periodic. These periods can be tracked to the points at which the heart valves are opening and shutting. If an HCP is interested in understanding the valvular health of a host, looking at heart function in association with an EKG can be very useful. The EKG can be used to identify a threshold event, upon which the acoustic sensor begins acoustic monitoring, i.e., collecting data. The EKG can, for example, identify when a mitral valve is functioning, which may be a threshold event. The EKG also identifies the periodicity of the heartbeat so that the IMD can estimate with high accuracy the periodicity of mitral valve function. With this information, the in vivo acoustic sensor device 100Y may respond by collecting data only during the time of mitral valve opening and shutting, which may be on the order of 100 to 200 milliseconds. If the heart of the host is beating at, e.g., 60 beats per minute, the IMD may be configured to activate the acoustic sensor only during the period of a heartbeat when the mitral valve is opening and closing, and not collect data during other times which may not be of primary interest. In this way, IMD power is conserved and the data of primary interest (reflecting mitral valve function, e.g., mitral valve prolapse) is collected exclusively. Alternatively, the in vivo acoustic sensor device 100Y may collect data continuously for a longer period of time, e.g., 10 seconds, at a sample rate of 5,000 Hz, and from this large amount of data the acoustic signal from the functioning of the mitral valve may be extracted. This later approach utilizes more power and requires that more information be stored in the memory.

In some embodiments, the EKG of the in vivo acoustic sensor device 100Y is used to identify threshold events, which triggers the collection of acoustic data by the in vivo acoustic sensor device 100Y. By combining the EKG information with the acoustic information, it is possible to obtain even higher fidelity data. The present technology allows for correlating an acoustic signature at a point in time during an EKG, in other words, evaluating heart performance by both EKG and acoustic signature for a particular point, or a family of particular points, in time. In some embodiments, the acoustic data is synched with EKG data, when the in vivo acoustic sensor device 100Y contains both an acoustic sensor and an EKG monitor.

In some embodiments, for example, the in vivo acoustic sensor device 100Y optionally includes a motion sensor. As used herein, a motion sensor is able to detect and/or measure the movement and orientation of the host relative to the earth gravitational force. As used herein, a motion sensor is able to detect and/or measure the position and/or movement of the host vis-à-vis the earth. For example, the motion sensor can detect whether a host is standing or laying down, walking or jogging or running, climbing stairs, swimming, or riding a bicycle, etc., and optionally take measurements that describe the motion, e.g., how fast is the host running, or is the host going upstairs or downstairs? An accelerometer such as a triaxial accelerometer, an inclinometer, and an inertial measurement unit (IMU), are each an exemplary motion sensor for this purpose.

The motion sensor may be used to confirm or ensure the acoustic data is collected during certain activities or activity levels. For example, the motion sensor may obtain measurements consistent with the host taking a walk, whereupon the acoustic sensor of the IMD is activated in order to obtain acoustic data. For example, when the motion sensor detects that the host is walking or jogging or running, such as occurs during a stress test when the host moves on a treadmill, the acoustic sensor may begin taking measurements. For example, the acoustic sensor may listen for sounds coming from the beating heart, which will provide information about how the heart is functioning during walking or jogging or running. In this way, acoustic data may be correlated with host position or host movement data. In some embodiments, the acoustic data is synched with position and/or movement data, when the in vivo acoustic sensor device 100Y contains both an acoustic sensor and a motion sensor.

Thus, the motion sensor may be used to confirm or ensure the acoustic data is collected during certain activities or activity levels. An accelerometer, inclinometer, inertial measurement unit (IMU), or preferably a triaxial accelerometer are all embodiments of a motion sensor for this purpose. To evaluate position and/or movement of the patient, in general, the accelerometer for the IMU can be configured as a low fidelity accelerometer, as opposed to a high-fidelity accelerometer which may be used for detecting and measuring internal (in vivo) acoustic signals, e.g., such as an acoustic accelerometer as described herein.

Other examples of auxiliary sensors include fluid pressure sensors, fluid volume sensors, pulse pressure sensors, blood volume sensors, blood flow sensors, chemistry sensors (e.g., for blood and/or other fluids), metabolic sensors (e.g., for blood and/or other fluids), pH sensors, analyte sensor.

### Electronics Unit 120Y

Example embodiments of the in vivo acoustic sensor device 100Y may include electronic components in addition to the acoustic sensor, e.g., the acoustic accelerometer, and/or the electronic components associated with a membrane that forms a microphone, and the one or more auxiliary sensor(s). The following are some exemplary electronic components of the electronics unit 120Y, in which the exemplary electronic components may be optional for some embodiments of the in vivo acoustic sensor device 100Y.

In some embodiments, the electronics unit 120Y may have a power supply configured to, e.g., generate a regulated supply signal in an approximate range of 1-24 Volts (V), to power the power-consuming components of the IMD. The power supply may include one or more of a battery, a primary battery or primary cell (a galvanic cell) that is designed to be used once and discarded without being recharged with electricity, a rechargeable power device (e.g., a rechargeable battery or a super capacitor) which may be recharged, e.g., inductively, or an energy harvester.

The power supply may be any suitable battery, such as a Lithium Carbon Monofluoride (LiCFx) battery, or other storage cell configured to store energy for powering components of the electronics assembly for an expected lifetime (e.g., 2 to 25+ years) of the IMD. Optionally, the power supply may be a rechargeable power device, such as a lithium-ion battery or a supercapacitor. In this case, the power supply includes additional components for charging the power source by an external recharge unit. These additional components may include a power coil configured to generate a voltage and current in response to a near magnetic field generated by an external recharge unit.

As another option, the power supply may include an energy harvester. The energy harvester is configured to convert an environmental stimulus into an energy for charging a rechargeable power device. For example, the harvester may convert, into a battery-charging electrical current or voltage or a supercapacitor-charging, one or more of body heat from the subject in which the IMD is implanted, kinetic energy generated by the subject's movement, changes in pressure (e.g., barometric pressure or pressure within the subject, such as the subject's blood pressure), energy generated by an electrochemical reaction within the subject's body, energy generated by radio-frequency (RF) fields, and light.

In some embodiments, the electronics unit 120Y includes a processor, such as a microcontroller. An exemplary microcontroller can be any suitable microcontroller or microprocessor and is configured to control the configuration and operation of one or more of the other electronic components. For example, the microcontroller may be configured to control the one or more sensors of the implanted IMD to sense relevant measurement data, and to store the measurement data generated by the one or more sensors in the memory. The microcontroller may also be configured to generate a message for communication over one or more types of communication interfaces. For example, in the case of RF telemetry communication, the microcontroller generates messages that include the stored data as a payload, packetizes the messages, and provides the message packets to an RF transceiver for transmission to the base station. The microcontroller may also be configured to execute commands received from a base station via a communication interface, e.g., an antenna, filter and RF transceiver. For example, the microcontroller can be configured to receive configuration data from the base station, and to provide the configuration data to a component of the electronics components to which the base station directed the configuration data. If the base station directed the configuration data to the microcontroller, then the microcontroller is configured to configure itself in response to the configuration data.

In some embodiments, the electronics unit 120Y includes a memory. The exemplary memory may include volatile memory and non-volatile memory. For example, the volatile memory may be configured to store the operating system and one or more applications executed by the microcontroller. The non-volatile memory may be configured to store configuration information for the IMD and to store data written by the microcontroller, and to provide data in response to a read command from the microcontroller.

In some embodiments, the electronics unit 120Y may include an antenna with an associated telemetry circuit, which may also be referred to as a communication interface. These components are configured to enable the implanted IMD to transmit and receive wireless data between the implanted device and an external device. The antenna and telemetry circuit may operate using a medical implant communication service (e.g., 400-405 MHz) or using Bluetooth (e.g., 2400-2500 MHz). In some embodiments, the antenna is a contained antenna, in other words, an antenna that is wholly contained within the housing 101Y of the in vivo acoustic sensor device 100Y.

In some embodiments, the electronics unit 120Y may include a communication interface which facilitates communication between the in vivo acoustic sensor device 100Y and another device or the in vivo fluid flow sensor device 100X of the IMD 100. For example, the other device may be, for example, an external device, e.g., a base station, that is located outside of or away from the patient who has received the IMD, or it may be an internal device that is located in the patient who has received the IMD. Example modes of intra-body communication include: (i) RF telemetry communication, (ii) tissue conductive communication, e.g., galvanic coupling communication, and/or (iii) data-over-sound communication, e.g., ultrasound or acoustic communication.

The communication interface includes communication circuitry that is generally, but not necessarily, associated with the electronics unit 120Y of the in vivo acoustic sensor device 100Y. The communication circuitry may include any hardware, firmware, software or any combination thereof suitable for enabling one or more modes of intra-body communication. To this end, the communication circuitry may include, for example, voltage regulators, current generators, oscillators, or circuitry for generating a signal, resistors, capacitors, inductors, and other filtering circuitry for processing received signals, as well as circuitry for modulating and/or demodulating a signal according to a communication protocol.

Depending on the mode of communication, the communication circuitry may also include transistors or other switching circuitry for selectively coupling transmitted signals to or receiving signals from a desired transceiver, such as an antenna (which may be used for electromagnetic communication, e.g., RF telemetry communication) or electrodes (which may be used for tissue conductive communication) or an acoustic transducer (which may be used for data-over-sound communication). Under the control of the microcontroller, communication circuitry may receive downlink communication signals from, as well as send uplink communication signals to, an external device or another implanted device. In addition, communication circuitry may communicate with a networked computing device via an external device and a computer network, such as the CareLink® Network. Additional details on each of the RF telemetry communication, tissue conductive communication, and data-over-sound communication modes of intra-body communication follow.

An RF telemetry mode of intra-body communication is enabled by an RF communication interface that includes an antenna and RF telemetry circuitry, e.g., an RF transceiver and a filter. The RF transceiver can be a conventional transceiver that is configured to allow the microcontroller (and optionally fuse) to communicate with another implanted medical device, or with a base station configured for use with the implanted IMD. For example, the RF transceiver can be any suitable type of transceiver (e.g., Bluetooth, Bluetooth Low Energy, and Wi-Fi®), can be configured for operation according to any suitable protocol (e.g., MICS, ISM, Bluetooth, Bluetooth Low Energy, and Wi-Fi®), and can be configured for operation in a frequency band that is within a range of 1 MHz-5.4 GHZ, or that is within any other suitable range. The filter can be any suitable bandpass filter, such as a surface acoustic wave (SAW) filter or a bulk acoustic wave (BAW) filter. The antenna can be any antenna suitable for the frequency band in which the RF transceiver generates signals for transmission by the antenna, and for the frequency band in which a base station generates signals for reception by the antenna.

A tissue conductive communication (TCC) mode of intra-body communication can include a TCC interface that includes TCC circuitry and a pair of electrodes. The TCC interface allows the microcontroller to communicate with another device having a same TCC interface as the implanted IMD. The other device may be an implanted medical device, or a base station configured for use with the implanted IMD. TCC can be configured to rely on the ion content of body tissue of a patient within which the IMD has been implanted and is thus frequently referred to as galvanic communication. The ion content of the body tissue provides an electrical communication medium over which to send and receive information to and from the implanted IMD. To communicate in a transmit mode, the TCC circuitry applies a voltage across the electrodes to cause current to flow between the electrodes and a corresponding electrical signal to propagate through the body tissue of the host. The propagating current may be detected by a receiving device by measuring the voltage generated between two electrodes. To communicate in a receive mode, the TCC circuitry measures voltage across the electrodes.

A data-over-sound mode of intra-body communication can include a data-over-sound communication interface that includes data-over-sound circuitry and at least one acoustic transducer. The data-over-sound communication interface allows an exemplary processor (e.g., microcontroller) to communicate with another device having a same data-over-sound communication interface as the implanted IMD. The other device may be another implanted medical device, or a base station configured for use with an example embodiment of the IMD 100.

In some embodiments, for example, the electronic components of the electronics unit 120Y may include a fuse. The fuse may be any suitable fuse (e.g., permanent) or circuit breaker (e.g., resettable) configured to prevent the power supply or a current flowing from the power supply, from injuring the patient and/or damaging one or more electronic components of the in vivo acoustic sensor device 100Y. For example, the fuse can be configured to prevent the power supply from generating enough heat to burn the patient, to damage the electronic components or to damage structural components of the in vivo acoustic sensor device 100Y.

In some embodiments, for example, the electronic components of the electronics unit 120Y may include a clock in association with a power management unit. The clock and power management unit can be configured to generate a clock signal for one or more of the other components of the electronic components and can be configured to generate periodic commands or other signals (e.g., interrupt requests) in response to which an exemplary processor (e.g., microcontroller) causes one or more components of the electronic components to enter or to exit a sleep, or other low-power, mode. The clock and power management unit also can be configured to regulate the voltage from the power supply, and to provide a regulate power-supply voltage to some or all of the other electronic components of the in vivo acoustic sensor device 100Y. For example, in some embodiments, the electronic components of the electronics unit 120Y may include a real time clock (RTC).

In some embodiments, for example, the in vivo fluid flow device 100X and/or the in vivo acoustic sensor device 100Y of the IMD 100 comprises a hermetic seal. The hermetic seal may provide a barrier between one or more of the electronic components of the IMD, e.g., one or more sensors or memory, and the tissue sounding the implanted IMD. With the hermetic seal, the internal electronics are safe from degradation due to bodily fluids, and the host is safe from any harmful effects that might result if the electronics of the IMD were to contact the tissue or fluids of the host.

The housing 101Y that houses the in vivo acoustic sensor device 100Y has a mass density. In some embodiments, the mass density of the in vivo acoustic sensor device 100Y is comparable to the mass density of the tissue in which the in vivo acoustic sensor device 100Y has been implanted. In embodiments, the mass density of the in vivo acoustic sensor device 100Y is within 1%, or within 2%, or within 3%, or within 4%, or within 5%, or within 6%, or within 7%, or within 8%, or within 9%, or within 10% of the mass density of the tissue into which the in vivo acoustic sensor device 100Y has been implanted. In some embodiments, the mass density of the in vivo acoustic sensor device 100Y is less than the mass density of the surrounding tissue, i.e., the tissue into which the in vivo acoustic sensor device 100Y has been implanted. In embodiments, the mass density of the in vivo acoustic sensor device 100Y is 99% to <100% of the mass density of the surrounding tissue, or 98% to <100% of the mass density of the surrounding tissue, or 97% to <100% of the mass density of the surrounding tissue, or is 96% to <100% of the mass density of the surrounding tissue, or is 95% to <100% of the mass density of the surrounding tissue, or is 94% to <100% of the mass density of the surrounding tissue, or is 93% to <100% of the mass density of the surrounding tissue, or is 92% to <100% of the mass density of the surrounding tissue, or is 91% to <100% of the mass density of the surrounding tissue, or is 90% to <100% of the mass density of the surrounding tissue.

Much living tissue has mass density similar to water at 1 g/cc or 1000 kg/m³. In embodiments, the housing 101Y that houses the in vivo acoustic sensor device 100Y according to the present technology has a mass density of less than 1000 kg/m³, or less than 950 kg/m³, or less than 900 kg/m³, or less than 850 kg/m³, or less than 800 kg/m³, or less than 750 kg/m³, or has a mass density between any of these listed values, e.g., between 1000 kg/m³ and 950 kg/m³.

In some embodiments, the acoustic sensor 111Y of the in vivo acoustic sensor device 100Y is located within the housing 101Y that provides and maintains separation between the acoustic sensor 111Y and the host tissue in which the in vivo acoustic sensor device 100Y is implanted. The housing 101Y may be formed from a biocompatible material, which term includes one or more biocompatible materials. The biocompatible material will not cause harm to a host that comes into physical contact with the material or any degradation product of the material that forms when the material is contacted with the host in vivo. For example, neither the material itself nor its in vivo biodegradation products (if any) demonstrate harmful cytotoxicity, genotoxicity, mutagenicity, carcinogenicity or immunogenicity to the host. An exemplary biocompatible material is a biocompatible metal and metal alloys, e.g., titanium. An exemplary biocompatible material is a biocompatible polymer, e.g., a plastic such as polyether ether ketone (PEEK). The housing may be formed from a mixture of biocompatible materials, for example, both metal and plastic may be used to form the housing 101Y.

In some embodiments, the in vivo acoustic sensor device 100Y includes a first housing structure that contains a sensor, e.g., the acoustic sensor 111Y, and little or nothing else, which may be referred to herein as a tethered component, and a second housing structure that contains other components of the in vivo acoustic sensor device 100Y. Because the housing of the tethered component encloses very few components, e.g., only one or more sensors such as the acoustic sensor 111Y, the tethered component may be prepared to have a particularly small mass density. As mentioned elsewhere herein, it may be advantageous for an acoustic sensor in particular to be contained within a housing that has a lower mass density that the surrounding tissue. By attaching only one or more sensors to a housing, the resulting tethered component may be fabricated to have a particularly low mass density. In some embodiments, the tethered component is physically connected to a base component of the in vivo acoustic sensor device 100Y, e.g., the second housing structure. In such embodiments, for example, the base component of the in vivo acoustic sensor device 100Y contains the majority of the electronic components of the electronics unit 120Y, e.g., one or more of an antenna, a telemetry circuit, a memory configured to store sensor data, a power source, auxiliary sensor(s), etc. may be located within a housing of the base component of the in vivo acoustic sensor device 100Y. In some embodiments, the mass density of the base component is greater than the mass density of the tethered component.

In some embodiments, sensor(s) which have a performance that is not impacted by the mass density of the component in which they are located, may be located in the base component of the in vivo acoustic sensor device 100Y. For example, the performance of a motion sensor is not impacted by the mass density of the component to which the motion sensor is physically affixed. Accordingly, the motion sensor may be placed in the base component of the in vivo acoustic sensor device 100Y.

The base component may be physically connected to the tethered component by a lead that runs between the base component and the tethered component. The tethered component may be said to be tethered to the base component via the lead. The lead may allow sensor data obtained by the sensor(s) located in the tethered component to be transferred to the base component of the in vivo acoustic sensor device 100Y, e.g., to a memory located in the base component. In addition, the lead may allow power held in the base component to be transferred to the tethered component and to be available to activate and/or power the sensor(s) of the tethered component. In some embodiments, the lead is flexible. In some embodiments, the lead is biocompatible. As an analogy, the tethered component secured to a flexible lead is analogous to a fly-fishing rod, where the base component is analogous to the rod and reel to which the fly (analogous to the tethered component) is secured by way of the fishing line (analogous to the lead).

In some embodiments, the in vivo acoustic sensor device 100Y comprises one base component and one tethered component with a lead running therebetween. In some embodiments, the in vivo acoustic sensor device 100Y includes one base component and two tethered components. In some embodiments, the in vivo acoustic sensor device 100Y includes one base component, two tethered components, and two leads, where a lead runs between the base component and each of the two tethered components. In some embodiments, the in vivo acoustic sensor device 100Y includes a single base component and a plurality of tethered components. In some embodiments, the in vivo acoustic sensor device 100Y includes a single base component, a plurality of tethered components, and one or more leads that allow data from the tethered components to be transferred to the base component. In some embodiments, the in vivo acoustic sensor device 100Y includes a single base component, a plurality of tethered components where at least one of the plurality is directly physically associated with an accelerometer that functions as an acoustic sensor, and one or more leads that allow data from the tethered components to be transferred to the base component. In effect, the tethered component(s) decouple the mass of the entire in vivo acoustic sensor device 100Y, so that, e.g., an acoustic sensor of the in vivo acoustic sensor device 100Y may be physically associated with a relatively small mass (present as a tethered component).

In some embodiments, the in vivo acoustic sensor device 100Y includes a tethered component comprising an accelerometer configured to function as an acoustic sensor, where the accelerometer is completely enclosed by the housing. In addition, the in vivo acoustic sensor device 100Y includes a base component and a lead that runs between the base component and the tethered component. For example, the base component can include a housing that encloses one or more electrical components selected from a power source, an antenna, a telemetry circuit, and a memory to store sensor data. Optionally, the base component may also include an auxiliary sensor as disclosed herein, for example, one auxiliary sensor, or two auxiliary sensors, or three or more auxiliary sensor. For example, in some embodiments, the base component can include a motion sensor. As another example, in some embodiments, the base component can include an ECG sensor. As yet another example, in some embodiments, the base component can include an ultrasonic sensor assembly comprising one or more ultrasonic transducer elements. In yet another example, in some embodiments, the base component can include one or more of an auxiliary sensor including one or more of a temperature sensor, an analyte sensor, or a pressure sensor.

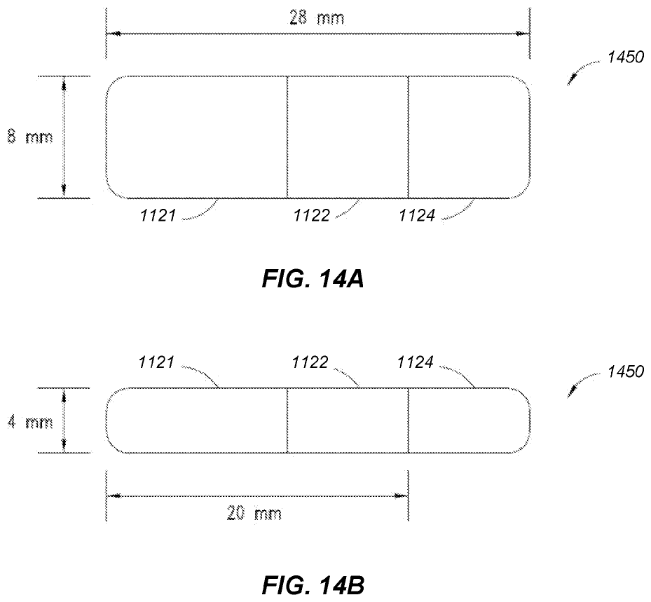

FIG. 14A shows a top view diagram showing an implantable structure of an example embodiment of the in vivo acoustic sensor device 100Y, labeled as IMD 1450, and indicating the location therein of internal components, specifically a battery 1121, an electronics package 1122 and an antenna 1124. In the IMD 1450, there is no tethered component. Each of the elements of the IMD 1450 is contained within a single housing, which in the diagram of FIG. 14A, is indicated to have a size of 28 mm (length) by 8 mm (width), where these are exemplary sizes only. In some embodiments, for example, the length of the IMD 1450 may be from about 25 mm to about 35 mm, e.g., about 25 mm, or about 26 mm, or about 27 mm, or about 28 mm, or about 29 mm, or about 30 mm. In some embodiments, the width of the IMD 1450 may be from about 6 mm to about 10 mm, e.g., about 6 mm, or about 7 mm, or about 8 mm, or about 9 mm, or about 10 mm. Notably, the housing may be made from more than one material. For example, the housing enclosing the antenna 1124 may be formed of radio-transmissive material, such as PEEK, so as to form a radome for the antenna, while the housing surrounding the battery 1121 and the electronics package 1122 may be formed from metal or plastic including PEEK.

FIG. 14B shows a side view diagram of the exemplary IMD 1450 of FIG. 14A. The side view diagram of the IMD 1450 indicates an exemplary size of 20 mm for the combined length of the battery 1121 and electronic package 1122, and an exemplary height of 4 mm of the IMD 1450. In some embodiments, for example, the height of the IMD 1450 may be from about 3 mm to about 5 mm, for example, about 3 mm, or about 4 mm, or about 5 mm.

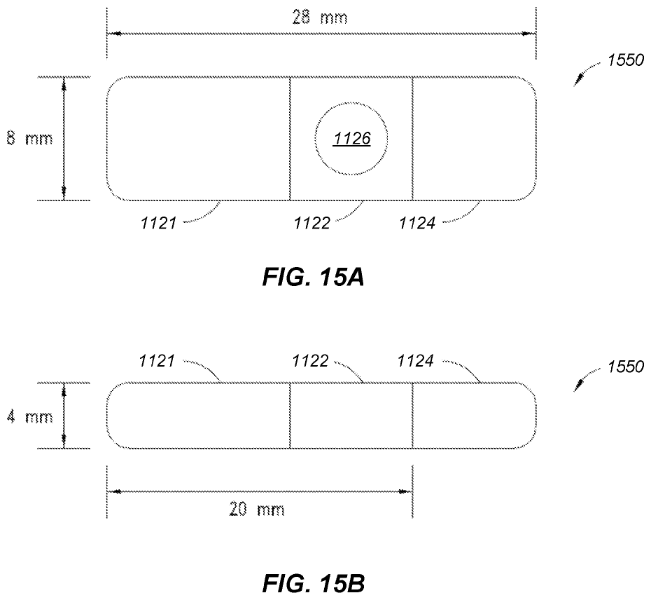

FIG. 15A shows a top view diagram showing an implantable structure of an example embodiment of the in vivo acoustic sensor device 100Y, labeled as IMD 1550, and indicating the location therein of internal components, specifically a battery 1121, an electronics package 1122 with a membrane 1126, and an antenna 1124. Features as discussed in FIG. 14A can apply to the IMD 1550 of FIG. 15A for various embodiments.

FIG. 15B shows a side view diagram of the exemplary IMD 1550 of FIG. 15A. Features as discussed in FIG. 14B can apply to the IMD 1550 of FIG. 15B for various embodiments.

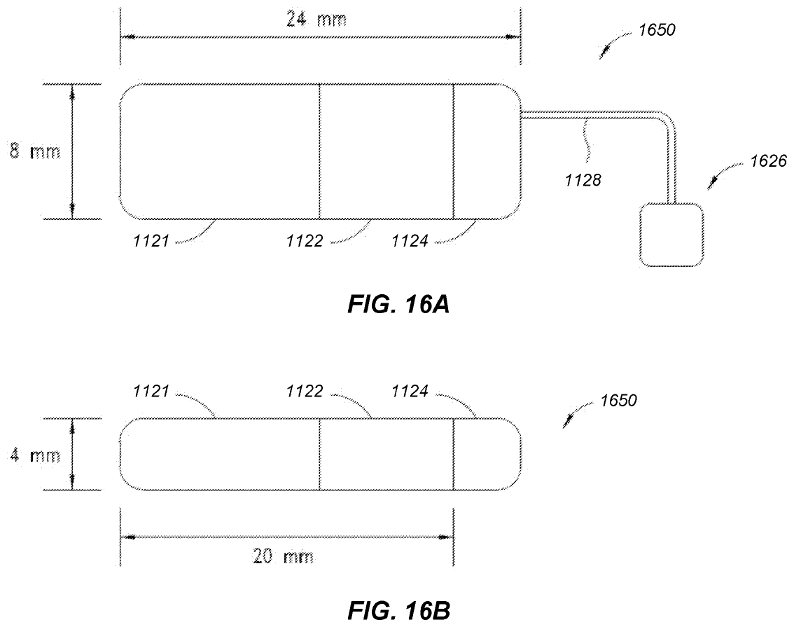

FIG. 16A shows a top view diagram showing an implantable structure of an example embodiment of the in vivo acoustic sensor device 100Y, labeled as IMD 1650, and indicating the location therein of internal components, specifically a battery 1121, an electronics package 1122 and an antenna 1124, where an accelerometer is contained within a tethered component 1626 connected to the main component by a lead 1128. Features as discussed in FIG. 14A can apply to the IMD 1650 of FIG. 16A for various embodiments.

FIG. 16B shows a side view of the exemplary IMD 1650 of FIG. 16A but omitting the tethered component 1626 and the associated lead 1128 in the drawing. Features as discussed in FIG. 14B can apply to the IMD 1650 of FIG. 16B for various embodiments.

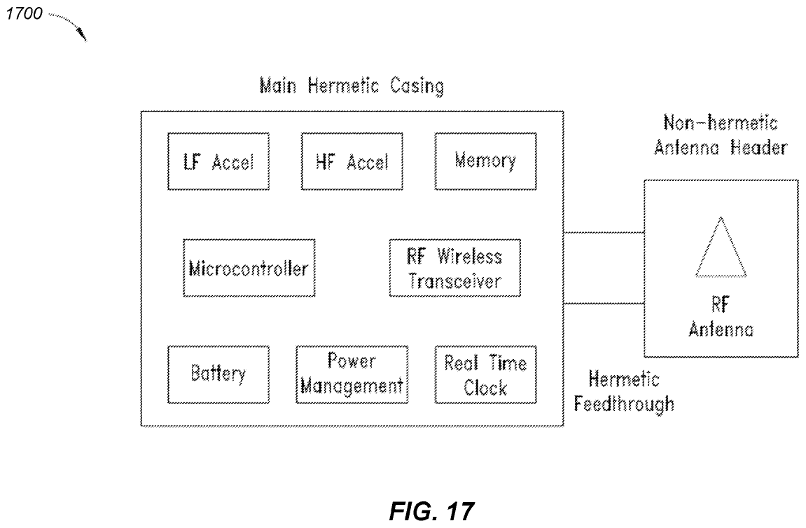

FIG. 17 shows a block diagram depicting an exemplary IMD including an example embodiment of the in vivo acoustic sensor device 100Y, labeled IMD 1700, including indications of optional internal components thereof. In the IMD 1700 of FIG. 17, the exemplary acoustic sensor is an accelerometer, denoted as the high fidelity (HF) Accel. Optional components of the IMD 1700 are also shown in FIG. 17, and may include a main hermetic casing, a low fidelity (LF) accelerometer which functions as an auxiliary sensor, and more specifically as a motion sensor. The optional components of the IMD 1700 as shown in FIG. 17 may include a memory to store sensor-derived data, a microcontroller, a RF wireless transceiver as a component of a telemetry assembly, a battery as an exemplary power supply, a power management circuit, and a real time clock. In some embodiments, for example, the IMD 1700 may include an RF antenna which is connected to the main hermetic casing by way of a hermetic feedthrough. The RF antenna may be enclosed within a hermetic casing, or it may be enclosed within a non-hermetic antenna header, as shown in FIG. 17. The exemplary IMD 1700 can include other components that have been discussed in connection with other embodiments of an IMD disclosed herein.

In some embodiments of the IMD 1700, for example, when all components of the IMD 1700 are contained within a single housing, which may be a composite housing (e.g., such as a metal which surrounds and protects the electronic components and a polymer which surrounds and protects the antenna), the IMD 1700 may be referred to as having a cartridge design.

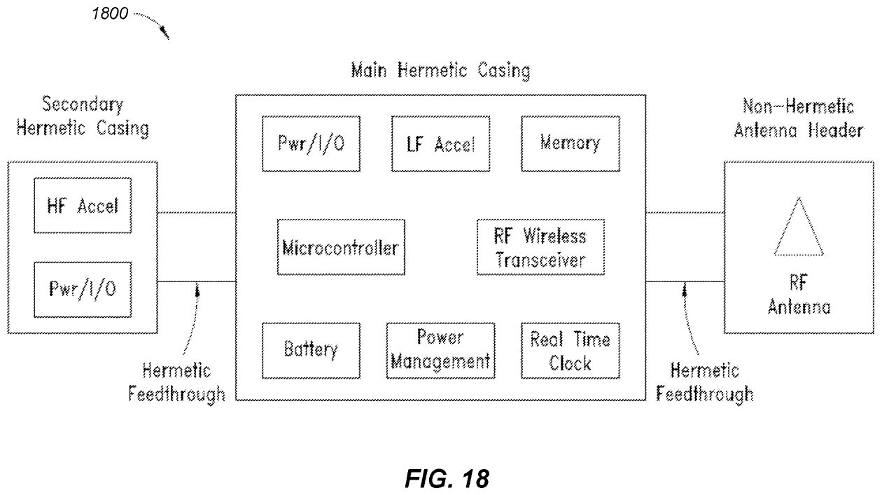

FIG. 18 shows a block diagram depicting an exemplary IMD including an example embodiment of the in vivo acoustic sensor device 100Y, labeled 1800, having a tethered component and including indications of internal components thereof. In the IMD 1800 of FIG. 18, the exemplary acoustic sensor is an accelerometer, denoted as the high fidelity (HF) accelerometer (also referred to as a vibration sensor, e.g., capable of measuring forces with ±1 g and/or ±10 g) capable of measuring and resolving accelerations as small as 100 micro-g, or 10 micro-g, or 1 micro-g, which is located in a tethered component of the IMD 1800 and contained within a secondary hermetic casing along with a power/I/O circuit. The tethered component is joined to the main component by way of a hermetic feed through, which is an exemplary lead, and which may be biocompatible. In the main component, optional components of the IMD 1800 are shown in FIG. 18, and may include a main hermetic casing, a Power/I/O, a low fidelity (LF) accelerometer which functions as an auxiliary sensor, and more specifically as a motion sensor. The optional components of the IMD 1800 as shown in FIG. 18 may include a memory to store sensor-derived data, a microcontroller, a RF wireless transceiver as a component of a telemetry assembly, a battery as an exemplary power supply, a power management circuit, and a real time clock. In some embodiments, for example the IMD 1800 may include an RF antenna which is connected to the main hermetic casing by way of a hermetic feedthrough. The RF antenna may be enclosed within a hermetic casing, or it may be enclosed within a non-hermetic antenna header as shown in FIG. 18. The exemplary IMD 1800 can include other components that have been discussed in connection with other embodiments of an IMD disclosed herein.

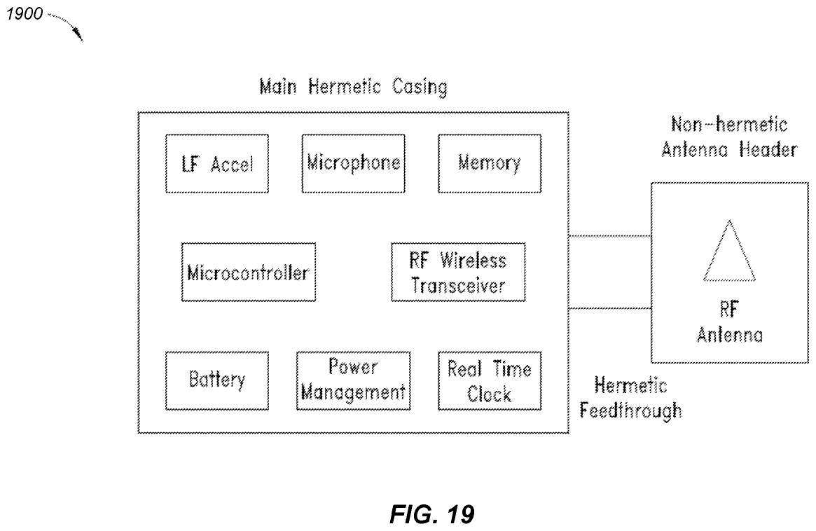

FIG. 19 shows a block diagram depicting an exemplary IMD including an example embodiment of the in vivo acoustic sensor device 100Y, labeled 1900, having a microphone as an acoustic sensor. Optional components of the IMD 1900 are also shown in FIG. 19, and may include a main hermetic casing, a low fidelity (LF) accelerometer which functions as an auxiliary sensor, and more specifically as a motion sensor. The optional components of the IMD 1900 as shown in FIG. 19 may include a memory to store sensor-derived data, a microcontroller to process data, a RF wireless transceiver as a component of a telemetry assembly, a battery as an exemplary power supply, a power management circuit, and a real time clock. In some embodiments, for example, the IMD 1900 may include an RF antenna which is connected to the main hermetic casing by way of a hermetic feedthrough. The RF antenna may be enclosed within a hermetic casing, or it may be enclosed within a non-hermetic antenna header, as shown in FIG. 19. The exemplary IMD 1900 can include other components that have been discussed in connection with other embodiments of an IMD disclosed herein.

In some embodiments, a housing of an IMD of the present technology includes securing aids that facilitate holding the IMD in a specific location in the host. For example, a housing of either or both of a main component and a tethered component may include rings that allow for suture to be used to secure the component to the tissue of a host. The ring is secured to, e.g., welded to, the outside of a housing, and a suture may be threaded through the ring and also through the tissue of a host in order to secure the housing, and accordingly the IMD, to the host at a selected location.

Some embodiments the present technology provides a method comprising one or more of: Manufacturing an IMD as described herein; Performing quality control on the IMD; Packaging the IMD for shipment or storage; Making the IMD available to a surgeon; Providing instructions for use of the IMD; Identifying a host having a medical condition in need of monitoring; Implanting the IMD into a host; Assisting a surgeon in implanting the IMD into a host; Operating robotics to assist a surgeon in implanting the IMD into a host; Transmitting a wireless signal to the implanted IMD and thereby activating the implanted IMD; Sensing acoustic signals generated by the host with the acoustic sensor of the implanted IMD, where optionally the acoustic signals are generated by one or more of the heart of the host, the blood flow of the host, the respiration of the host; Storing sensed acoustic signals or a derivative thereof in a memory located within the IMD to provide stored data, where optionally the stored data is stored in the memory for at least 1 hour, or at least 6 hours, or at least 12 hours, or at least 18 hours, or at least 24 hours, or at least 48 hours; Transmitting the stored data from the implanted IMD to a receiving device located outside of the host, to provide received data that is stored on a memory of the receiving device; Analyzing the received data to generate health information that is informative as to the physical condition of the host; Providing the health information to a health care provider (HCP); and Evaluating the health information as a component of developing a health care plan for the host.

In some embodiments of the in vivo acoustic sensor device 100Y, the present technology provides a method that includes measuring internal sounds with a medical implant and using those sounds to diagnose and/or detect and/or quantify the physical status of the host, e.g., the cardiac health and/or the respiratory health of the host. In some embodiments, the measurements are made in an acoustic spectrum range beginning at about 1 Hz, or 2 Hz, or 5 Hz, or 10 Hz, or 50 Hz, or 100 Hz, or 200 Hz, or 500 Hz. In some embodiments, the measurements are made in an acoustic spectrum range extending up to about 10,000 Hz, or up to about 8,000 Hz, or up to about 6,000 Hz, or up to about 4,000 Hz, or up to about 2,000 Hz, or up to about 1,000 Hz. In embodiments, the measurements are made in an acoustic spectrum range extending from 1 Hz to 2,000 Hz, or 10 Hz to 10,000 Hz, or extending from 50 Hz to 2000 Hz.

Example embodiments of the IMD 100 in accordance with the present technology may be placed (implanted) partially or entirely within the host. If only partially implanted within the host, then, in some embodiments, the IMD may include a tethered component that is entirely implanted within the host, where optionally the base component of the IMD may be located on the exterior of the host, or partially within and partially outside the host. In some embodiments, the IMD may include a base component and a tethered component, and the entire IMD is implanted within the host.

In some example embodiments of the IMD 100, the in vivo acoustic sensor device 100Y and/or the in vivo fluid flow sensor device 100X may be used to acoustically monitor and acoustically quantify medical conditions of a host. For example, the IMD 100 may monitor and quantify heart conditions such as heart disease, including valvular disease such as stenosis (narrowing), incontinence (leaking), and myxomatous, for any of the tricuspid, pulmonary, pulmonic, mitral or aortic valves. Myxomatous is particularly a problem for the mitral valve and is currently the most common form of valvular heart disease. The pathologic presentation of myxomatous mitral valve disease varies between valve thickness, degree of leaflet prolapse and the presence or absence of flail leaflets. Each of these different diseases for each of the heart valves, gives rise to a unique acoustic signature that may be detected with the IMD of the present technology. Other heart conditions which may be detected and evaluated acoustically with the IMD 100 include congestive heart failure, atrial fibrillation, coronary artery disease, and other detrimental heart-related medical conditions.

The IMD 100 may also, or alternatively, monitor and quantify respiratory conditions, such as pulmonary disease, chronic obstructive pulmonary disease (COPD, a condition involving constriction of the airways and difficulty or discomfort in breathing), emphysema, pulmonary embolism (PE), and asthma.

The IMD 100 is intended to be implanted in a host. For example, it may be implanted into a coelom of a host, into a ventral cavity of a host, into a dorsal cavity of a host, into a thoracic cavity of a host, into an upper ventral cavity of a host, into a lower ventral cavity of a host, into an upper dorsal cavity of a host, into a lower dorsal cavity of a host, into a pleural cavity of a host, into a pericardial cavity of a host, into an abdominopelvic cavity of a host, into an abdominal cavity of a host; into a pelvis of a host, into a spinal cavity of a host, or into a lower torso adipose tissue of a host. The IMD 100 may be implanted into and secured to the heart of a host, e.g., it may be secured across the septum of the heart between the two ventricles, or it may be in or secured to the left atrial appendage, i.e., inside the left atrium. Upon being implanted, it may operate to collect daily information reflecting the health status of the host. For instance, it may collect daily cardiovascular and/or respiratory acoustic signatures of the host when it is implanted in, e.g., the lower torso adipose tissue of a host.

The present technology also provides a method where an implanted IMD as described herein is removed from a host. The IMD may be removed for any of a variety of reasons, e.g., the host no longer needs the IMD to monitor a medical condition, or in order to make way for a different implanted device, or to provide a recharge for the battery, to name a few. The method comprises selecting a host with an implanted IMD as described herein, and performing surgery on the host to provide access to the implanted IMD, and then removing the implanted IMD from the host by way of the access created by the surgery.

In some embodiments, the in vivo acoustic sensor device 100Y includes both an acoustic sensor and a motion sensor, and the in vivo acoustic sensor device 100Y is configured so that acoustic information is obtained when the motion sensor detects a threshold event. For example, if the motion sensor detects that the host has started walking, where walking is a threshold event, the IMD is configured to activate the acoustic sensor to begin making measurements. In some embodiments, the in vivo acoustic sensor device 100Y waits to obtain acoustic data after the motion sensor detects threshold movement. In some embodiments, the acoustic data is obtained for 5 to 30 seconds. The threshold movement may be walking. The threshold movement may be a resting state. The in vivo acoustic sensor device 100Y thus operates to obtain acoustic data under controlled conditions, when the in vivo acoustic sensor device 100Y detects certain movement or non-movement of the host and then begins to collect acoustic data that has been influenced by that particular threshold activity performed by the host. For instance, when the host moves, this puts a stress on the host's body, and the cardiovascular system must respond by increasing output. Under these conditions, the sounds generated by the cardiovascular system may reflect health concerns that would otherwise not be apparent from acoustic signals obtained when the host is in resting state. This is the reason for a stress test, where a subject is asked to run on a treadmill while the HCP listens to the host's heart.

The information gathered from example embodiments of the IMD 100 implanted in a host may be used to inform clinical decision-making. For example, the information may characterize the effectiveness of ongoing treatment, possibly provoking the HCP to consider and implement alternative treatments. The information may characterize disease progression which upon consideration by a HCP, may provoke the HCP to alter, e.g., escalate, the treatment protocols. In addition to providing valuable information for the HCP, the information from the implanted IMD may reduce the need for emergency room visits by the host due to early detection of congestive heart failure and possible non-compliance of drug therapy. In effect, example embodiments of the IMD 100 affords an HCP with a “remote stethoscope” for daily remote monitoring of the medical condition of the patient, e.g., the health of the patient's cardiovascular system.

### Stress-Mediated In Vivo Acoustic Sensor Device

In some embodiments of the in vivo acoustic sensor device 100Y, for example, the acoustic sensor 111Y includes a stress-mediated acoustic sensor. An exemplary stress-mediated acoustic sensor in accordance with the present technology is configured to measure stress—not displacement—to provide in vivo sensing of acoustic energy (mechanical waves) that emanate from within the body, e.g., from anatomical structures of the cardiovascular system and/or pulmonary system, such as the heart and lungs. The disclosed stress-mediated acoustic sensor is operable to detect an in vivo acoustic signal by measuring applied mechanical force upon the electromechanical transducer component of the sensor to convert the received mechanical energy (acoustic signal) into electrical energy (electrical signal), which is addressable and processible to decipher the physiological phenomenon associated with the detected acoustic signal and to determine clinically relevant information about the patient's health and/or disease. In some embodiments of the stress-mediated acoustic sensor, the transducer component includes a piezoelectric material and electrically-conductive non-piezoelectric material(s) configured in a piezoelectric sensing unit.

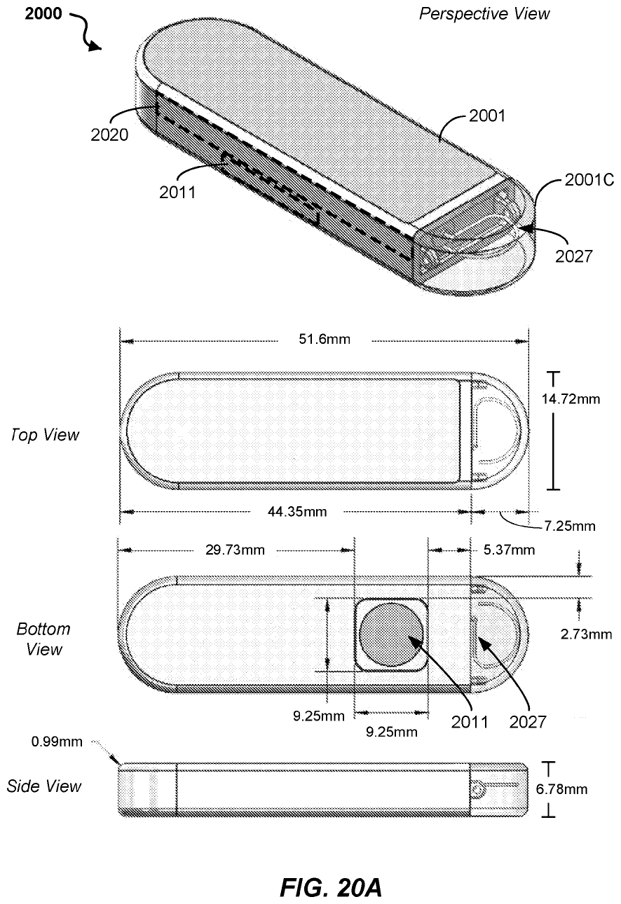

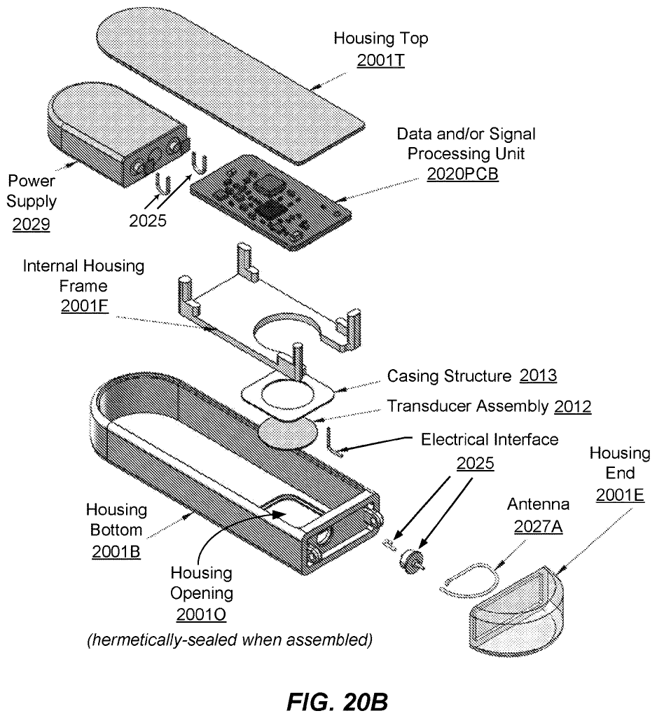

FIGS. 20A and 20B show diagrams depicting an exemplary IMD of the present technology, labeled 2000, having a stress-mediated acoustic sensor. FIG. 20A shows multiple views of the exterior of the IMD 2000 depicting an exemplary body structure; and FIG. 20B shows an exploded view of the IMD 2000 depicting an example configuration of the components of the IMD 2000.

FIG. 20A shows a perspective view illustrating an exemplary body of the IMD 2000, showing a housing 2001 that hermetically-seals within a sensor unit of the IMD 2000, which includes stress-mediated acoustic sensor 2011 (illustrated in broken lines), and an electronics unit 2020 (illustrated in broken lines) in communication with the stress-mediated acoustic sensor 2011. The perspective view diagram of FIG. 20A also show an example embodiment of a wireless communication unit 2027, e.g., embodied as a wire antenna, which is disposed in a distal chamber 2001C of the housing 2001, e.g., to facilitate wireless communication transmission(s) and/or reception(s) between the IMD 2000 and an external device (e.g., the external remote device 130).

Also shown in FIG. 20A are a top view, a bottom view, and a side view of the exemplary body of the IMD 2000, which illustrates one example of the shape, size and dimensions of an example embodiment of the housing 2001 of the IMD 2000. It is understood that the IMD 2000 can be configured to have a range of sizes and shapes to accommodate the stress-mediated acoustic sensor 2011 and electronics unit 2020 contained therein, which are beyond the particular sizes, dimensions, and shapes shown in FIG. 20A for the example IMD 2000. In the non-limiting example shown in FIG. 20A, the end-to-end length of the housing 2001 is configured to be 51.6 mm, the end-to-end width of the housing 2001 is configured to be 14.72 mm, and the end-to-end height of the housing 2001 is configured to be 6.78 mm. Other non-limiting example shapes for the IMD 2000 and for various portions of the housing 2001 are contemplated, including rectangular, square, triangular, elliptical, circular, cylindrical, conical, or others or combinations thereof or of others.

FIG. 20B shows an exploded view of an example embodiment of the IMD 2000, featuring components of the stress-mediated acoustic sensor 2011 and the electronics unit 2020 that are hermetically sealed within the housing 2001, which includes a first chamber enclosed by a housing top 2001T and a housing bottom 2001B and an adjacent second chamber (i.e., distal chamber 2001C) enclosed by housing end 2001E. In some embodiments of the IMD 2000, for example, the stress-mediated acoustic sensor 2011 includes transducer assembly 2012 that is coupled to a casing structure 2013, which is configured to secure the transducer assembly 2012. For example, in some embodiments of the stress-mediated acoustic sensor 2011, the transducer assembly 2012 includes a piezoelectric material and at least one electrically-conductive non-piezoelectric material coupled to the piezoelectric material to receive the transduced electrical signals generated upon applied stress to the stress-mediated acoustic sensor 2011 that is received by the piezoelectric material. In some embodiments, for example, the at least one electrically-conductive non-piezoelectric material is positioned on an interior-facing side of the piezoelectric material and coupled to electrical interface component(s) 2025. In some embodiments, for example, the electrical interface components can be configured in or on the casing structure 2013. In some embodiments, for example, the casing structure 2013 or a portion thereof can include at least a portion of the at least one electrically-conductive non-piezoelectric material to receive and transfer the generated electrical signals. In some embodiments, for example, the transducer assembly 2012 includes a non-piezoelectric material (not shown in FIG. 20B) that is positioned on an exterior-facing side of the piezoelectric material and to be disposed at the housing opening 20010 (i.e., opening through a side of housing bottom 2001B), which is hermetically-sealed when assembled, where the non-piezoelectric material has a Young's modulus close to (e.g., within 2×-3× of) or substantially matching (e.g., within 20% of) that of the piezoelectric material.

In some embodiments of the IMD 2000, for example, the electronics unit 2020 includes a data and/or signal processing unit 2020PCB, which can be embodied by any of the exemplary embodiments of the data processing unit 121Y and/or the optional signal conditioning unit 123Y, respectively, disclosed herein. In the example shown in FIG. 20B, the data and/or signal processing unit 2020PCB includes a processor coupled to a memory which receives digital signal data from a signal processing circuit, all mounted on a printed circuit board (PCB). In some example embodiments, for example, the signal processing circuit includes a differential amplifier and/or charge amplifier to amplify the electrical signals received from the stress-mediated acoustic sensor 2011 (e.g., from the at least one electrically-conductive non-piezoelectric material via the electrical interface component(s) 2025 that connect the stress-mediated acoustic sensor 2011 to the electronics unit 2020), and/or an analog-to-digital (A/D) converter to digitize the electrical signals. In some example embodiments, for example, the signal processing circuit includes filter circuit(s) to remove signal outside frequency range(s) of non-interest, which can include low-pass, bandpass, and/or high-pass filters, e.g., to improve signal-to-noise ratio of the detected acoustic signal of interest. The exemplary data and/or signal processing unit 2020PCB can be programmable for some embodiments. In some embodiments, for example, the data and/or signal processing unit 2020PCB can be secured and/or positioned within the first chamber of the housing 2001 by an (optional) internal housing frame 2001F, which may also be used to secure and/or position one or more components of the stress-mediated acoustic sensor 2011 for some example embodiments.

In some embodiments of the IMD 2000, for example, the electronics unit 2020 includes a power supply 2029 electrically connected, via components (e.g., wires or other connectors) of the electrical interface 2025, to the data and/or signal processing unit 2020PCB and other components of the electronic unit 2020 (or other unit of the IMD 2000, such as some embodiments of the stress-mediated acoustic sensor 2011, when electrical power is needed to be supplied to such units). The power supply 2029 can be embodied by any of the exemplary embodiments of the power supply 129 disclosed herein. For example, the power supply 2029 can include a battery (e.g., primary or rechargeable), fuel cell, or other power source to supply power to the components of the electronics unit 2020 (and, optionally, the stress-mediated acoustic sensor 2011). In some example embodiments, for example, the power supply 2029 can be an electrical receiving port to receive a wire that can supply the IMD 2000 from a remote power source, e.g., such an implantable (in vivo) power supply (e.g., a battery associated with one or more other implanted medical device(s)) and/or a wearable (in vitro) power supply (e.g., a battery worn by the user with the wire connecting the battery to another in vivo device implanted into the body of the patient).

In some embodiments of the IMD 2000, for example, the electronics unit 2020 includes a wireless communications unit 2027 electrically connected, via components (e.g., wires or other connectors) of the electrical interface 2025, to the data and/or signal processing unit 2020PCB and other components of the electronic unit 2020 (or other unit of the IMD 2000, such as some embodiments of the stress-mediated acoustic sensor 2011, e.g., for transmitting raw electrical signals transduced by the acoustic sensor). The wireless communication unit 2027 can include a wireless transmitter, receiver, and/or transceiver device, e.g., antenna 2027A as shown in FIG. 20B, which is capable of communicating with an external device to communicate raw, partially-processed, or fully-processed data from the data and/or signal processing unit 2020PCB. For example, the wireless communications unit 2027 can be configured to manage the communication protocol for transmission or reception via the antenna. The wireless communication unit 2027 can be embodied by any of the exemplary embodiments of the wireless communication unit 127Y disclosed herein. Examples of the antenna 2027A can include, but are not limited to, a whip antenna, a loop antenna, a chip antenna, a planar inverted F antenna (PIFA), a bipolar antenna, and/or a conformal antenna.

### Unimorph Piezoelectric Sensor

In some embodiments of the in vivo acoustic sensor 100Y, for example, the exemplary stress-mediated acoustic sensor includes a unimorph structure for sensing stress, comprising a piezoelectric material coupled to a biocompatible, electrically-conductive, non-piezoelectric material with a similar Young's modulus to the piezoelectric material, providing a piezoelectric sensing unit that generates a measurable electrical signal proportionate to an applied stress (force or moment) on the unimorph structure, and which is electrically addressable by a receiving circuit (e.g., for signal processing and/or data processing). Such a stress-mediated unimorph-structured piezoelectric acoustic sensor (also referred to herein as a “unimorph piezoelectric sensor”) is substantially less sensitive to any potential tissue overgrowth and biofouling due to (i) the stress sensing modality that does not require displacement of the transducing component and (ii) the material selection of the tissue-facing outer (non-piezoelectric) material component to minimize granulation tissue formation (e.g., from an immune response to implantation of the IMD). Furthermore, the unimorph piezoelectric sensor is capable of exhibiting substantially lower noise and substantially higher acoustic resolution for improved sensitivity to dynamic range, e.g., as compared to a displacement-mediated acoustic sensor, such as an electret microphone or condenser microphone. In some embodiments, for example, the stress-mediated acoustic sensor includes a charge amplifier circuit to condition the electrical signal generated by the piezoelectric material in various embodiments of the stress-mediated acoustic sensor, including but not limited to the unimorph piezoelectric sensor.

In some embodiments of the unimorph piezoelectric structure, the device structure includes an active piezoelectric material (e.g., piezoelectric film) capable of imparting or detecting a stress and a stable, non-piezoelectric material (e.g., metal substrate) disposed on a side of the active piezoelectric material; which, after a load is applied on the unimorph piezoelectric structure (e.g., on the metal substrate), it causes a bending moment (stress) that propagates to the active piezoelectric film, thereby generating an electrical signal corresponding to the applied stress that is detectable as a mechanical force sensor, i.e., capable to detect acoustic signals (mechanical waves). In some embodiments of the acoustic sensor 111Y, the piezoelectric material of the exemplary unimorph piezoelectric sensor can include, but is not limited to, PZT (e.g., PZT-5A, PZT-5H, or PZT-5K), PLZT, quartz, ZnO, AlN, ScAlN, BaTiO₃, PbTiO₃, KNbO₃, LiNbO₃, LiTaO₃, and/or Na₂WO₄. In some embodiments of the acoustic sensor 111Y, the non-piezoelectric material of the exemplary unimorph piezoelectric sensor can include, but is not limited to, titanium (Ti), a biocompatible stainless-steel, cobalt-chromium, nitinol, or high-purity ceramic (e.g., alumina Al₂O₃).

In example embodiments of the IMD 100, e.g., such as the IMD 2000 of FIGS. 20A and 20B, the unimorph piezoelectric sensor can be integrated in a biocompatible, hermetically-sealed housing structure for longevity and safety when implanted into a patient, where the non-piezoelectric component (e.g., Ti) is positioned on the device housing (or forms the device housing) and is exposed to the outer environment (tissue-facing); and where piezoelectric component (e.g., PZT material) is not exposed on the outer environment of the IMD 100, but is instead contained within the hermetically-sealed interior of the IMD. In such embodiments, for example, the piezoelectric material and non-piezoelectric material are selected and engineered such that the unimorph piezoelectric sensor is sufficiently sensitive to receive and transduce low amplitude acoustic signals of a large frequency range, i.e., having large dynamic range. For example, the unimorph piezoelectric sensor is capable of measuring and resolving sound levels as low as 20 dB SPL (e.g., barely above threshold of human hearing) and as high as 110 dB SPL (e.g., approaching human pain threshold).

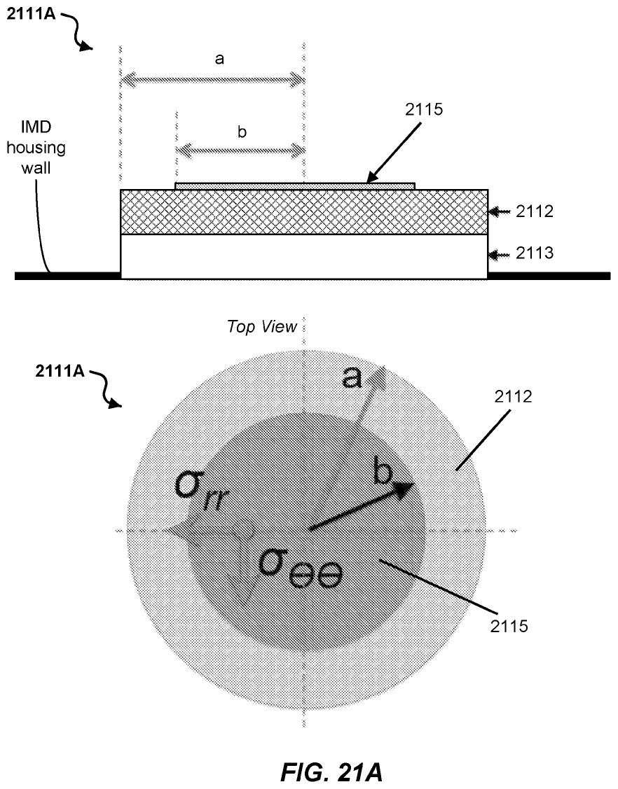

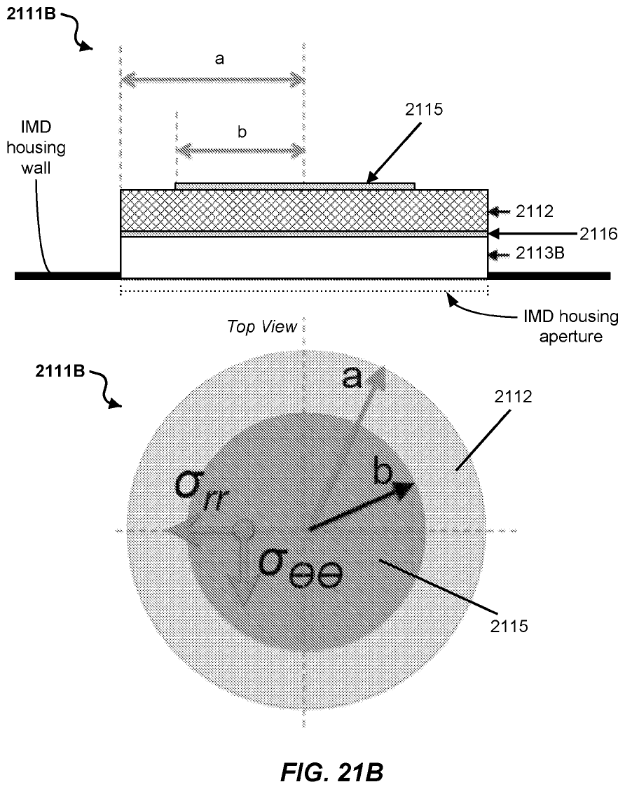

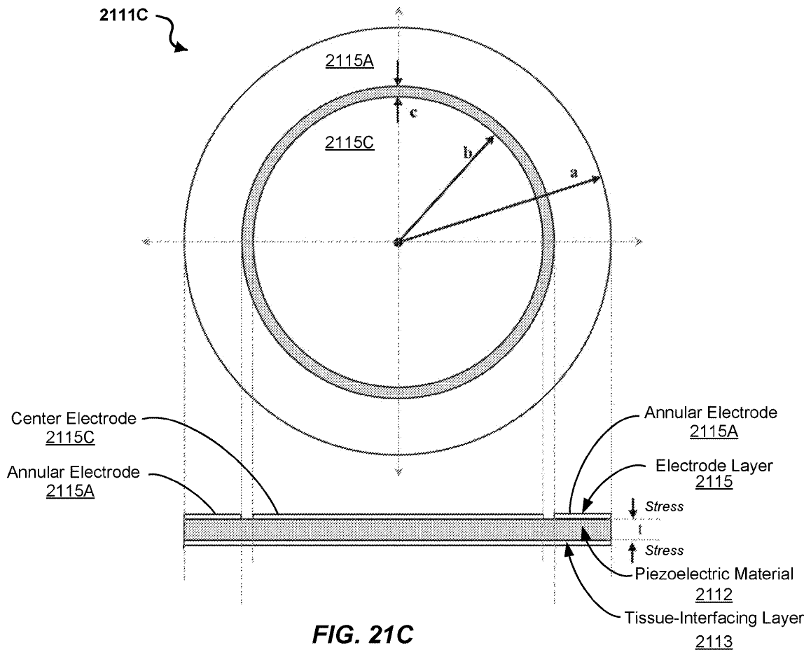

FIGS. 21A-21C show diagrams depicting example embodiments of a unimorph piezoelectric sensor device, labeled 2111A, 2111B and 2111C, respectively, in accordance with the present technology. FIG. 21A shows an example embodiment of the unimorph piezoelectric sensor device having at least one electrode electrically-interfaced with a piezoelectric transducer material coupled to a biocompatible, electrically-conductive, non-piezoelectric material; FIG. 21B shows an example embodiment of the unimorph piezoelectric sensor device having at least one electrode electrically-interfaced with a piezoelectric transducer material coupled to a biocompatible, non-electrically-conductive, non-piezoelectric material with an intermediary electrically-conductive material layer therebetween; and FIG. 21C shows an example embodiment of the unimorph piezoelectric sensor device having at least two electrodes electrically-interfaced with a piezoelectric transducer material.

As shown in FIG. 21A, the unimorph piezoelectric sensor device 2111A includes a piezoelectric material 2112 disposed between and coupled to a first layer 2113 and a second layer 2115. The first layer 2113 includes a biocompatible, electrically conductive, non-piezoelectric material and has a Young's modulus close within to (e.g., within 2×-3× of) or substantially the same as (e.g., within 20% of) the Young's modulus of the piezoelectric material 2112. In implementations of the unimorph piezoelectric sensor device 2111A, the first layer 2113 is configured to be a sensing layer that receives the mechanical wave emanated from within the patient's body, such that the applied force on the first layer 2113 is transferred through and into the piezoelectric material 2112, which acts as transduce the mechanical energy (stress) into electrical energy. The first layer 2113 is positioned in the unimorph piezoelectric sensor device 2111A to be at the aperture of the IMD, where the first layer 2113 is tightly coupled to the IMD housing wall in order to hermetically seal the other components of the unimorph piezoelectric sensor device 2111A within the enclosure. The second layer 2115 includes a non-piezoelectric, electrically conductive material. In implementations of the unimorph piezoelectric sensor device 2111A, the second layer 2115 provides at least one electrically-addressable electrode to receive the electrical signal generated from the piezoelectric material 2112; and the electrically conductive material of the first layer 2113 provides an electrically-addressable electrode for the piezoelectric sensing unit. Moreover, for some embodiments of the unimorph piezoelectric sensor device 2111A, because the material of the first layer 2113 is biocompatible, the housing of the IMD (e.g., housing wall(s)) can be configured as the first layer 2113, thereby allowing the piezoelectric material 2112 (and second layer 2115) to be manufactured on a region of the housing structure of the IMD.

In some embodiments of the unimorph piezoelectric sensor device 2111A, for example, the piezoelectric material includes PZT (e.g., PZT-5A, PZT-5H, or PZT-5K), the first layer 2113 includes titanium, and the second layer 2115 includes at least one of titanium, gold, platinum, silver, copper, nickel, aluminum, palladium, or other conductive material, including an alloy thereof. In such example embodiments where the first layer 2113 is electrically conductive, biocompatible, and mechanically matched (e.g., close Young's modulus) to the piezoelectric material 2112, such as Titanium is with PZT-5A, for example, the first layer 2113 can be coupled to the electronics unit 2020, as is the non-piezoelectric, electrically conductive material of the second layer 2115, so that both sides of the piezoelectric material 2112 can connect to an amplifier circuit to amplify the transduced electrical signal, i.e., the captured charge generated by the piezoelectric material 2112 in response to the applied stress. The amplifier circuit can be configured as a charge amplifier (or other type of amplifier) which conditions, processes, and passes the acoustic/stress signal for higher-order electronic functions by the IMD, e.g., such as data processing, data storage, wireless transmission, or other.

Yet, in some example embodiments of a unimorph piezoelectric sensor device of the disclosed technology, the first layer 2113 can be configured of a material that is biocompatible but not electrically conductive, where, in such embodiments, an intermediary electrically conductive layer is included for the unimorph piezoelectric sensor device, i.e., coupled to the piezoelectric material 2112 and the first layer 2113.

FIG. 21B shows an example embodiment of the unimorph piezoelectric sensor device 2111B that includes the piezoelectric material 2112 disposed between (i) a first layer 2113B comprising a biocompatible, non-electrically conductive, non-piezoelectric material and (ii) the electrically-conductive, non-piezoelectric second layer 2115, where the piezoelectric material 2112 is coupled to the second layer 2115 and an intermediary layer 2116 comprising an electrically conductive, non-piezoelectric material. Also shown in the example of FIG. 1B, the first layer 2113B is positioned in the unimorph piezoelectric sensor device 2111B to be at an aperture of the IMD housing, where the first layer 2113B is tightly coupled to the IMD housing wall in order to hermetically seal the other components of the unimorph piezoelectric sensor device 2111A within the enclosure. Like the first layer 2113 of FIG. 21A, the first layer 2113B of FIG. 21B includes a biocompatible, non-piezoelectric material having a Young's modulus close within to (e.g., within 2×-3× of) or substantially the same as (e.g., within 20% of) the Young's modulus of the piezoelectric material 2112. In implementations of the unimorph piezoelectric sensor device 2111B, the at least one electrode of the second layer 2115 and the electrically conductive material of the intermediary layer 2116 and electrically interfaced with the piezoelectric material 2112 and an amplifier circuit (e.g., of the electronics unit 2020) to receive the transduced electrical signal generated from the piezoelectric material 2112 for signal processing at the amplifier.

The unimorph piezoelectric sensor device 2111A and 2111B can be configured in a variety of shapes and geometries or the IMD within which it is employed. In the examples shown in FIGS. 21A and 21B, the unimorph piezoelectric sensor device 2111A and 2111B are configured with a cylindrical geometry and a circular-shaped sensor profile, as illustrated by the lower diagram showing a top side of the device 2111A and 2111B that is contained within the housing of the IMD, where the at least one electrically-addressable electrode 2115 is centrally positioned on the piezoelectric material 2112 (e.g., the electrode 2115 has a radius b, and the piezoelectric material 2112 has a radius a). The lower diagram of FIGS. 21A and 21B shows stress vectors Orr and gee, i.e., radial stress vector and/or tangential stress vectors, that radiate from center or tangentially, respectively. The stress vectors add linearly to produce a net polarization (charge) in the piezoelectric material 2112.

FIG. 21C shows an example embodiment of the unimorph piezoelectric sensor device 2111C that includes a circular/cylindrical shape/geometry having two electrode structures of the second layer 2115: center electrode 2115C and annular electrode 2115A that is positioned around and separated from the center electrode 2115C by a gap c. The gap c provides an electrical discontinuity between the two electrode structures 2115C and 2115A to create a voltage differential (potential) across the two electrodes, such that when a mechanical wave is incident (i.e., applied force or moment) on the first layer 2113 (i.e., tissue-interfacing layer 2113 in an IMD), the compressive stress caused by the applied force or moment propagates through the piezoelectric material 2112, which generates electric fields due to dipoles in the material structure causing different electrical potentials at the two electrode structures, resulting an addressable electrical signal across the center electrode 2115C and the annular electrode 2115A for the IMD.

The circular electrode, cylindrical shape of the example unimorph piezoelectric device 2111C shown in FIG. 21C possesses many advantages, including (but not limited to) the capability to provide an even distribution of stress in the materials of the unimorph piezoelectric device 2111C, so that there are no “hotspots” which can nucleate fracture/crack propagation; as well as reliability and safety for the device 2111, especially because the example unimorph piezoelectric device 2111C is part of a hermetic enclosure for an implantable device, where the circular/cylindrical configuration of the example unimorph piezoelectric device 2111C mitigates against potential degradation issues, such as corners that create stress risers and/or nucleation sites for cracks.

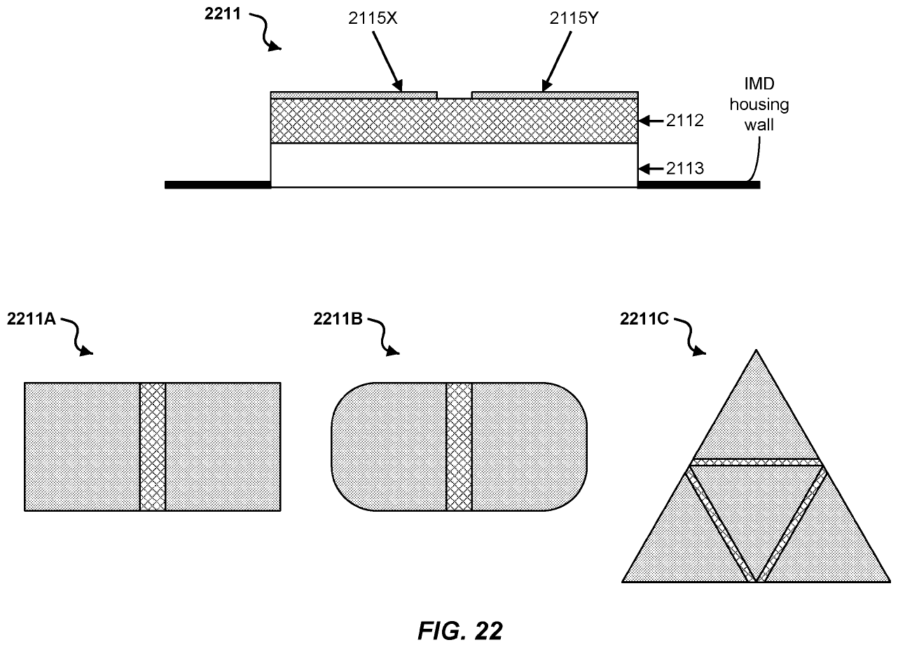

FIG. 22 shows diagrams depicting example geometries of example embodiments of the unimorph piezoelectric sensor device of FIG. 21A, labeled as 2211 in FIG. 22. The unimorph piezoelectric sensor device 2211 includes at least two electrode structures of the second layer 2115, shown in the top diagram of FIG. 22 as first electrode 2115X and second electrode 2115Y, which are separated from each other by a gap, and are coupled to the piezoelectric material 2112 that is coupled to the first layer 2113 (e.g., positioned in an IMD's housing to be exposed through an aperture). As depicted in lower diagrams 1211A, 1211B, and 1211C of FIG. 12, the at least two electrodes of the unimorph piezoelectric sensor device 1211 can be configured in a variety of geometries and configurations, including but not limited to rectangular, elliptical, and triangular, and can include three or more electrodes.

### Integrated/Interconnected In Vivo Fluid Flow Sensor and In Vivo Acoustic Sensor

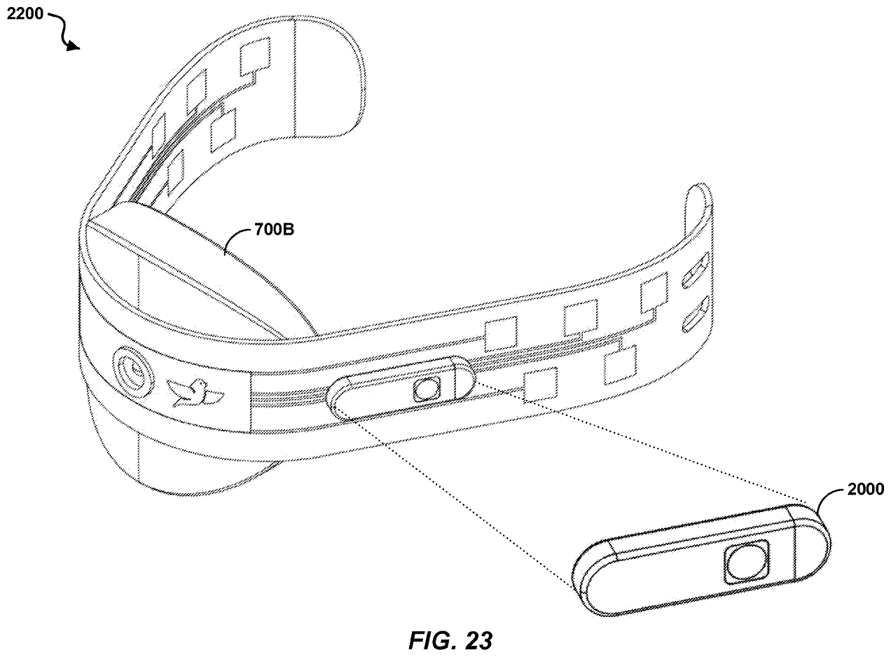

FIG. 23 shows a diagram illustrating an example embodiment of the implantable medical device 100, labeled as IMD 2300, depicting an integrated device comprising an example embodiment of the in vivo fluid flow sensor device 100X (in this exemplary embodiment, as the in vivo fluid flow sensor device 700B) and an example embodiment of the in vivo acoustic sensor device 100Y (i.e., in this exemplary embodiment, as the in vivo acoustic sensor device 2000). In this example, the in vivo acoustic sensor device 2000 is coupled to the clip band 703 of the in vivo fluid flow sensor device 700B, such that the electronics unit 2020 of the device 2000 is in electrical and/or data communication with the electronics unit 720 of the device 700B. For example, in some embodiments of the IMD 2300, the power supply of the IMD 2300 can be configured in only one of the electronics unit 2020 or electronics unit 720; or similarly, the data processing unit of the IMD 2300 may be configured in only one of the electronics unit 2020 or electronics unit 720; and/or other components of the electronics unit 2020 and the electronics unit 720 can be shared and/or consolidated. Also, for example, the in vivo acoustic sensor device 2000 is in data communication with the in vivo fluid flow sensor device 700B, allowing data communication between the electronics unit 2020 of the device 2000 and the electronics unit 720 of the device 700B, which can be used for synchronized data collection and processing protocols of the IMD 2300.

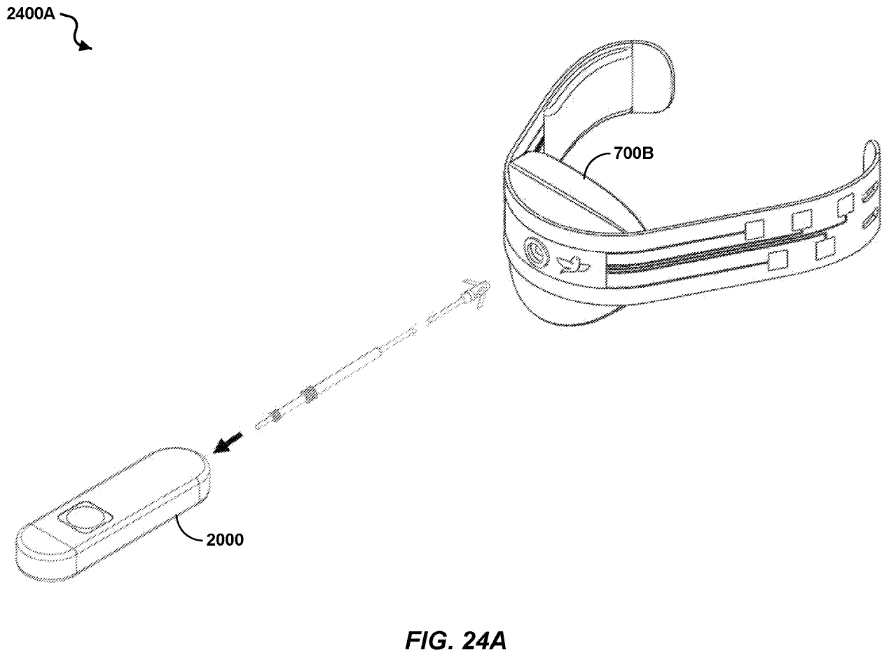

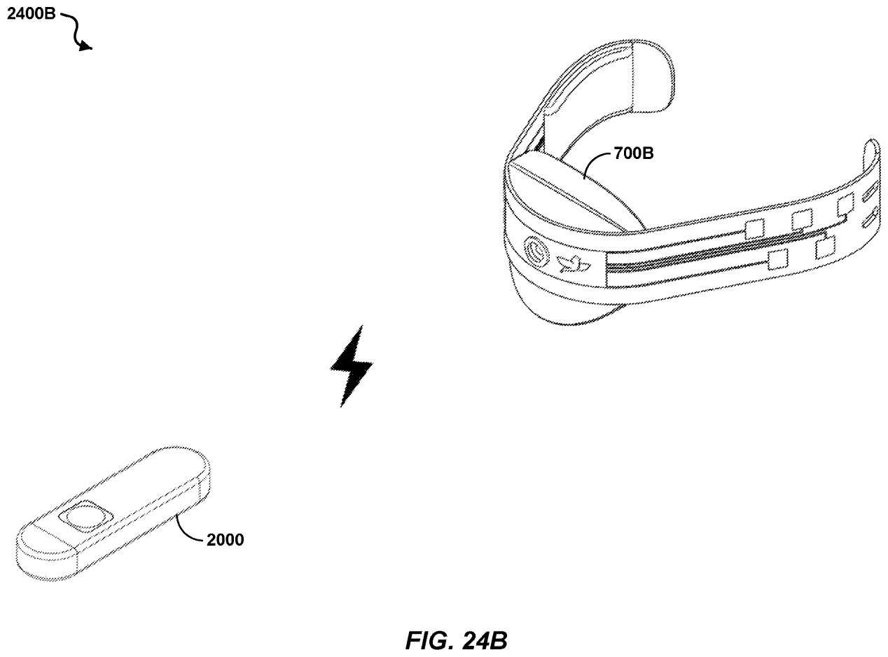

FIG. 24A shows a diagram illustrating an example embodiment of the implantable medical device 100, labeled as IMD 2400A, depicting an electrically and communicatively interconnected device comprising an example embodiment of the in vivo fluid flow sensor device 100X (in this exemplary embodiment, as the in vivo fluid flow sensor device 700B) and an example embodiment of the in vivo acoustic sensor device 100Y (i.e., in this exemplary embodiment, as the in vivo acoustic sensor device 2000). In this example, the in vivo acoustic sensor device 2000 is electrically and/or communicatively connected to the clip band 703 of the in vivo fluid flow sensor device 700B via a cable, wire, or cord (e.g., such as a cable, wire, or cord 993 of the remote in vivo device 990). In some embodiments of the IMD 2400A, for example, because the electronics unit 2020 of the device 2000 can be in electrical and/or data communication with the electronics unit 720 of the device 700B, various components of the respective electronics units can be shared and/or consolidated. In this example, the in vivo acoustic sensor device 2000 is in data communication (wired) with the in vivo fluid flow sensor device 700B, allowing data communication between the electronics unit 2020 of the device 2000 and the electronics unit 720 of the device 700B, which can be used for synchronized data collection and processing protocols of the IMD 2400A.

FIG. 24B shows a diagram illustrating an example embodiment of the implantable medical device 100, labeled as IMD 2400B, depicting a wirelessly communicative interconnected device comprising an example embodiment of the in vivo fluid flow sensor device 100X (in this exemplary embodiment, as the in vivo fluid flow sensor device 700B) and an example embodiment of the in vivo acoustic sensor device 100Y (i.e., in this exemplary embodiment, as the in vivo acoustic sensor device 2000). In this example, the in vivo acoustic sensor device 2000 is in wireless communication with the in vivo fluid flow sensor device 700B, allowing data communication between the electronics unit 2020 of the device 2000 and the electronics unit 720 of the device 700B, which can be used for synchronized data collection and processing protocols of the IMD 2400B.

### EXAMPLES

In some embodiments in accordance with the present technology (example A1), a sensor device for in vivo monitoring of fluid flow in an anatomic structure includes a first ultrasound assembly comprising a first set of one or more acoustic transducer elements; a second ultrasound assembly comprising a second set of one or more acoustic transducer elements; a first linkage coupled to the first ultrasound assembly; a second linkage coupled to the second ultrasound assembly; an electronics unit in electrical communication with the first ultrasound assembly and the second ultrasound assembly, the electronics unit configured to process the electrical signals associated with the returned acoustic signals as data and wirelessly transmit the data to an external processor; and a spring connection apparatus that couples to each of the first and second linkages and is operable to position the first ultrasound assembly at a first location on the anatomic structure and position the second ultrasound assembly at a second location on the anatomic structure to form a plane across the first and second locations of the anatomic structure to transmit and receive acoustic signals from the first set of one or more acoustic transducer elements and the second set of one or more acoustic transducer elements indicative of a fluid flow parameter of a biological fluid in the anatomic structure.
Example A2 includes the sensor device of any of examples A1-A4, wherein the anatomic structure is a heart.
Example A3 includes the sensor device of any of examples A1-A4, wherein the first location and the second location are positioned on a left atrium of the heart, and wherein the plane across which the acoustic signals are transmitted and received crosses at a mitral valve of the heart.
Example A4 includes the sensor device of any of examples A1-A3, wherein the first location and the second location are positioned on a right atrium of the heart, and wherein the plane across which the acoustic signals are transmitted and received crosses at a tricuspid valve of the heart.

In some embodiments in accordance with the present technology (example B1), a sensor device for in vivo monitoring of fluid flow in an anatomic structure includes a linkage assembly comprising a first arm configured to attach to a first portion of the anatomic structure and a second arm configured to attach to a second portion of the anatomic structure opposite to the first portion; a connection apparatus coupled to each of the first arm and the second arm; an ultrasound sensor assembly comprising a plurality of acoustic transducer elements coupled to the linkage assembly, the plurality of acoustic transducer elements including a first acoustic transducer element that is configured to transmit an acoustic signal to propagate through the anatomic structure and a second acoustic transducer element and a third acoustic transducer element that are configured to receive acoustic signals that have propagated through the anatomic structure and are indicative of a fluid flow parameter of a biological fluid in the anatomic structure; and an electronics unit housed in the connection apparatus and in electrical communication with the plurality of acoustic transducer elements of the ultrasound sensor assembly, the electronics unit configured to process electrical signals associated with the received acoustic signals as data and wirelessly transmit the data to an external processor.
Example B2 includes the sensor device of any of examples B1-B50, wherein the ultrasound sensor assembly includes a first ultrasound sensor assembly disposed on the first arm of the linkage assembly and a second ultrasound sensor assembly disposed on the second arm of the linkage assembly.
Example B3 includes the sensor device of example B2 or any of examples B1-B50, wherein the first ultrasound sensor assembly includes the first acoustic transducer element configured to transmit the acoustic signal that is positioned on a distal region of the first arm to interface with the first portion of the anatomic structure, wherein the second ultrasound sensor assembly includes the second and third acoustic transducer elements configured to receive the acoustic signals that are positioned on a distal region of the second arm to interface with the second portion of the anatomic structure, and wherein a first center point (C_{Tx}) of the first acoustic transducer element is aligned with a second center point (Cd_{Rx}) located between a distance (d) separating centers of the second acoustic transducer element and the third acoustic transducer element.
Example B4 includes the sensor device of example B2 or any of examples B1-B50, wherein the first ultrasound sensor assembly includes the first acoustic transducer element configured to transmit the acoustic signal that is positioned on a distal region of the first arm to interface with the first portion of the anatomic structure, wherein the second ultrasound sensor assembly includes the second and third acoustic transducer elements configured to receive the acoustic signals that are positioned on a distal region of the second arm to interface with the second portion of the anatomic structure, wherein the first ultrasound sensor assembly further includes a fourth acoustic transducer element and a fifth acoustic transducer element positioned on the distal region of the second arm to interface with the second portion of the anatomic structure and configured to receive a first set of acoustic signals associated with transmission of the acoustic signal by the first acoustic transducer element, wherein the second ultrasound sensor assembly includes a sixth acoustic transducer element positioned on the distal region of the first arm to interface with the first portion of the anatomic structure and configured to transmit a second acoustic signal associated with the acoustic signals received at the second and third acoustic transducer elements, and wherein a first center point (C_{Tx1}) of the first acoustic transducer element is aligned with a second center point (Cd_{Rx1}) located between a first distance (d1) separating centers of the fourth acoustic transducer element and the fifth acoustic transducer element, and wherein a third center point (C_{Tx2}) of the sixth acoustic transducer element is aligned with a fourth center point (Cd_{Rx2}) located between a second distance (d2) separating centers of the second acoustic transducer element and the third acoustic transducer element.
Example B5 includes the sensor device of any of examples B1-B50, wherein the plurality of acoustic transducer elements of the ultrasound sensor assembly is disposed on the first arm of the linkage assembly, and wherein the sensor device further comprises a reflector disposed on the second arm of the linkage assembly.
Example B6 includes the sensor device of any of examples B1-B50, wherein at least one of the first acoustic transducer element, the second transducer element, or the third acoustic transducer element is configured to have a size dimension between 1 mm to 4 mm.
Example B7 includes the sensor device of any of examples B1-B50, wherein the ultrasound sensor assembly further comprises a substrate connected to an interior-facing surface of at least one of the first arm or the second arm of the linkage assembly and that couples at least one of the plurality of acoustic transducer elements.
Example B8 includes the sensor device of example B7 or any of examples B1-B50, wherein the substrate is configured to provide a heat sync for management of thermal generation by the at least one acoustic transducer element.
Example B9 includes the sensor device of any of examples B1-B50, wherein at least one of the plurality of acoustic transducer elements includes a piezoelectric micromachined ultrasonic transducer (PMUT).
Example B10 includes the sensor device of any of examples B1-B50, wherein the sensor device further comprises an acoustic transducer pad that couples to at least some of the acoustic transducer elements of the ultrasound sensor assembly and is configured to provide a contour and cushion against the anatomic structure.
Example B11 includes the sensor device of example B10 or any of examples B1-B50, wherein the acoustic transducer pad includes a hydrogel.
Example B12 includes the sensor device of any of examples B1-B50, wherein the linkage assembly is operable to secure the sensor device to the anatomic structure with pliability for the sensor device to be stable in its placement with respect to the anatomic structure while withstanding continuous movements of the anatomic structure to which its attached.
Example B13 includes the sensor device of example B12 or any of examples B1-B50, wherein the linkage assembly includes a clip band operable to provide a compression force by each of the first arm and the second arm of the clip band to facilitate securement of the sensor device to the first portion and the second portion of the anatomic structure and to absorb forces applied onto the first arm and the second arm of the clip band by the anatomic structure due to the continuous movements of the anatomic structure.
Example B14 includes the sensor device of example B13 or any of examples B1-B50, wherein the clip band comprises a composite material having a polymer component that is flexible and a shape-stiffener component that is pre-shaped and is capable of undergoing a shape change.
Example B15 includes the sensor device of example B14 or any of examples B1-B50, wherein the shape-stiffener component of the composite material comprises one or more of Nitinol, gold, platinum, or iridium, which is encased in the polymer component of the composite material that comprises one or more of silicone, polyethylene, polyimide, polyamide, or a blend thereof.
Example B16 includes the sensor device of any of examples B1-B50, wherein the connection apparatus is operable to secure the linkage assembly to the anatomic structure with mechanical resilience for the sensor device to be stable in its placement with respect to the anatomic structure while withstanding continuous movements of the anatomic structure to which its attached.
Example B17 includes the sensor device of example B16 or any of examples B1-B50, wherein the connection apparatus includes a spring operable to provide a compression force upon each of the first arm and the second arm of the linkage assembly to facilitate securement of the sensor device to the first portion and the second portion of the anatomic structure and to absorb forces applied onto the first arm and the second arm of the linkage assembly by the anatomic structure due to the continuous movements of the anatomic structure.
Example B18 includes the sensor device of any of examples B1-B50, wherein the electronics unit comprises a power supply and a wireless communications unit including a wireless transmitter or wireless transceiver.
Example B19 includes the sensor device of example B18 or any of examples B1-B50, wherein the power supply includes at least one of a battery or a fuel cell.
Example B20 includes the sensor device of example B18 or any of examples B1-B50, wherein the electronics unit comprises a signal conditioning unit in communication with the plurality of acoustic transducer elements of the ultrasound sensor assembly via one or more electrical interface components, the signal conditioning unit comprising an electrical circuit configured to process the electrical signals associated with the received acoustic signals by one or more of amplifying the electrical signals, filtering the electrical signals, or converting the electrical signals from analog to digital.
Example B21 includes the sensor device of example B20 or any of examples B1-B50, wherein the electronics unit comprises a data processing unit in communication with the signal conditioning unit, the data processing unit comprising a processor and a memory and configured to process the amplified, filtered, or converted electrical signals as the data representative of the fluid flow parameter of the biological fluid.
Example B22 includes the sensor device of example B18 or any of examples B1-B50, wherein the electronics unit comprises a data processing unit in communication with the wireless communications unit, the data processing unit comprising a processor and a memory and configured to process the electrical signals as the data representative of the fluid flow parameter of the biological fluid.
Example B23 includes the sensor device of example B18 or any of examples B1-B50, wherein the electronics unit comprises a printed circuit board (PCB) having a substrate and electrical interconnections disposed on the substrate, wherein the electrical interconnections are coupled to a plurality of electrical interconnection wires that span between the plurality of acoustic transducer elements of the ultrasound sensor assembly and the PCB of the electronics unit.
Example B24 includes the sensor device of example B23 or any of examples B1-B50, wherein the electronics unit comprises a casing that encompasses the electronics unit to protect the electronics unit from exposure to a body fluid when the sensor device is inserted and deployed in vivo.
Example B25 includes the sensor device of example B24 or any of examples B1-B50, wherein the casing includes one or both of flat sides or curved sides to provide a form factor of the sensor device, including at least one of rectangular, a cylindrical, a conical, an elliptical, a pyramidal, a trapezoidal, or a non-uniform shape.
Example B26 includes the sensor device of example B24 or any of examples B1-B50, wherein the casing includes is coupled to the linkage assembly on an inward-facing surface of the linkage assembly that faces toward the anatomic structure, or wherein the casing includes is coupled to the linkage assembly on an outward-facing surface of the linkage assembly that faces away from the anatomic structure.
Example B27 includes the sensor device of example B24 or any of examples B1-B50, wherein the PCB of the electronics unit is hermetically sealed within the casing by a non-permeable material that covers the PCB to provide an electrical shield from the body fluid.
Example B28 includes the sensor device of example B27 or any of examples B1-B50, wherein the non-permeable material includes at least one of a parylene, a urethane, or a Teflon material.
Example B29 includes the sensor device of any of examples B1-B50, further comprising a secondary sensor coupled to at least one of the linkage assembly or the connection apparatus and in communication with the electronics unit, the secondary sensor operable to measure one or more of a biological parameter, a physiological parameter, an electrophysiological parameter, or a physical parameter of the body within which the sensor device is deployed.
Example B30 includes the sensor device of example B29 or any of examples B1-B50, wherein the secondary sensor includes an analyte sensor configured to detect an analyte of the biological fluid in the anatomic structure or of a body fluid in a region proximate the anatomic structure where the sensor device is deployed.
Example B31 includes the sensor device of example B29 or any of examples B1-B50, wherein the secondary sensor includes a pH sensor configured to detect a pH level of the biological fluid in the anatomic structure or of a body fluid in a region proximate the anatomic structure where the sensor device is deployed.
Example B32 includes the sensor device of example B29 or any of examples B1-B50, wherein the secondary sensor includes a temperature sensor configured to detect temperature of the biological fluid in the anatomic structure or of a body fluid in a region proximate the anatomic structure where the sensor device is deployed.
Example B33 includes the sensor device of example B29 or any of examples B1-B50, wherein the secondary sensor includes an inertial measurement unit (IMU) configured to detect motion of the sensor device in multiple degrees of freedom.
Example B34 includes the sensor device of any of examples B1-B50, further comprising a secondary attachment component comprising one or more of a suture, a prong, a screw, a barb, an adhesive, or a grasping mechanism disposed on at least one portion of the linkage assembly and configured to secure the to the ultrasound sensor assembly via the linkage assembly to the anatomic structure.
Example B35 includes the sensor device of any of examples B1-B50, wherein the linkage assembly is capable of changing shape from a first shape conformation, where the first arm and the second arm of the linkage assembly spread outward from a centerline through at least one of the linkage assembly or the connection apparatus, to be insertable into a body of a patient, to a second shape conformation, where the first arm and the second arm of the linkage assembly span inward toward the centerline through at least one of the linkage assembly or the connection apparatus, to be attachable to the anatomic structure for operation of the sensor device.
Example B36 includes the sensor device of any of examples B1-B50, further comprising a second linkage assembly comprising a third arm configured to attach to a third portion of the anatomic structure and a fourth arm configured to attach to a fourth portion of the anatomic structure opposite to the third portion, wherein the connection apparatus is coupled to each of the third arm and the fourth arm, and wherein the ultrasound sensor assembly comprises an additional set of the plurality of acoustic transducer elements coupled to the second linkage assembly and configured to transmit a second acoustic signal to propagate through the anatomic structure across the third portion and the fourth portion and to receive a second set of acoustic signals that have propagated through the anatomic structure across the third portion and the fourth portion and are indicative of a second fluid flow parameter of the biological fluid in the anatomic structure.
Example B37 includes the sensor device of any of examples B1-B50, wherein the connection apparatus includes a port in electrical communication with the electronics unit housed in the connection apparatus and operable to electrically couple to a remote power supply housed in a remote device via at least one of a cable, wire, or cord, wherein the remote device is located in another location different than and at a distance from the anatomic structure.
Example B38 includes the sensor device of example B37 or any of examples B1-B50, wherein the port is in data communication with the electronics unit housed in the connection apparatus and operable to interface the electronics unit to a data processing unit disposed in the remote device via the at least one of the cable, wire, or cord to output the data from the electronics unit of the sensor device to the data processing unit of the remote device through the port via the at least one of the cable, wire, or cord.
Example B39 includes the sensor device of example B38 or any of examples B1-B50, wherein the sensor device is configured to wirelessly transmit the data by a secondary transmission device comprising a wireless transmitter or transceiver to the external processor.
Example B40 includes the sensor device of example B37 or any of examples B1-B50, wherein the other location where the remote device is located is at least 2 cm from the anatomic structure.
Example B41 includes the sensor device of example B37 or any of examples B1-B50, wherein the other location includes a pleural cavity, a cavity in the abdominal space, a subcutaneous space, or an outer-body location.
Example B42 includes the sensor device of any of examples B1-B50, wherein the external processor that is able to receive the data to be wirelessly transmitted by the electronics unit of the sensor device is located outside of the body of a patient user to which the sensor device is implantable and deployable at the anatomic structure.
Example B43 includes the sensor device of any of examples B1-B50, wherein the fluid flow parameter of the biological fluid in the anatomic structure includes at least one of a flow rate, or an amount of pressure difference between ends of a flow path in the anatomic structure.
Example B44 includes the sensor device of example B43 or any of examples B1-B50, wherein the device is operable to detect a size dimension of the anatomic structure or a predictive area or volume of the anatomic structure based on a baseline measurement of the flow rate and changes in the flow rate over time.
Example B45 includes the sensor device of any of examples B1-B50, wherein the anatomic structure is a heart, and the biological fluid is blood.
Example B46 includes the sensor device of example B45 or any of examples B1-B50, wherein the sensor device is deployable within at least one layer of the pericardium of the heart, or wherein the sensor device is deployable on an external layer of the pericardium of the heart.
Example B47 includes the sensor device of example B45 or any of examples B1-B50, wherein the first arm and the second arm of the linkage assembly are positioned on opposing portions of a left atrium of the heart, and wherein the received acoustic signals are indicative of blood flow at a mitral valve of the heart.
Example B48 includes the sensor device of example B45 or any of examples B1-B50, wherein the first arm and the second arm of the linkage assembly are positioned on opposing portions of a right atrium of the heart, and wherein the received acoustic signals are indicative of blood flow at a tricuspid valve of the heart.
Example B49 includes the sensor device of example B45 or any of examples B1-B50, wherein the first arm and the second arm of the linkage assembly are positioned on a superior vena cava or an inferior vena cava proximate the heart, and wherein the received acoustic signals are indicative of blood flow at the superior vena cava or the inferior vena cava.
Example B50 includes the sensor device of example B45 or any of examples B1-B49, wherein the first arm and the second arm of the linkage assembly are positioned on a pulmonary artery or pulmonary vein proximate the heart, and wherein the received acoustic signals are indicative of blood flow at the pulmonary artery or the pulmonary vein.

In some embodiments in accordance with the present technology (example B51), a sensor device for in vivo monitoring of blood flow in a heart or blood vessel leading into or out of the heart in a body of a patient, includes, a linkage assembly comprising a first arm configured to attach to a first portion of the heart or the blood vessel and a second arm configured to attach to a second portion of the heart or the blood vessel; a connection apparatus coupled to each of the first arm and the second arm; an ultrasound sensor assembly comprising a plurality of acoustic transducer elements coupled to the linkage assembly, the plurality of acoustic transducer elements including a first acoustic transducer element that is configured to transmit an acoustic signal to propagate through the heart or the blood vessel and a second acoustic transducer element and a third acoustic transducer element that are configured to receive acoustic signals that have propagated through the heart or the blood vessel and are indicative of a fluid flow parameter of the blood that flows in the heart or blood vessel; and an electronics unit housed in the connection apparatus and in electrical communication with the plurality of acoustic transducer elements of the ultrasound sensor assembly, the electronics unit configured to process electrical signals associated with the received acoustic signals as data and wirelessly transmit the data to an external processor, wherein the linkage assembly includes a clip band operable to provide a compression force by each of the first arm and the second arm of the clip band to facilitate securement of the sensor device to the first portion and the second portion of the heart or the blood vessel and to absorb forces applied onto the first arm and the second arm of the clip band by heartbeats or changes in size of the heart or the blood vessel.
Example B52 includes the sensor device of any of examples B51-B60, wherein the clip band comprises a composite material having a polymer component that is flexible and a shape-stiffener component that is pre-shaped and is capable of undergoing a shape change.
Example B53 includes the sensor device of example B52 or any of examples B51-B60, wherein the shape-stiffener component of the composite material comprises one or more of Nitinol, gold, platinum, or iridium, which is encased in the polymer component of the composite material that comprises one or more of silicone, polyethylene, polyimide, polyamide, or a blend thereof.
Example B54 includes the sensor device of any of examples B51-B60, wherein the ultrasound sensor assembly includes a first ultrasound sensor assembly disposed on the first arm of the clip band and a second ultrasound sensor assembly disposed on the second arm of the clip band, or wherein the plurality of acoustic transducer elements of the ultrasound sensor assembly is disposed on the first arm of the clip band, and wherein the sensor device further comprises a reflector disposed on the second arm of the clip band.
Example B55 includes the sensor device of any of examples B51-B60, wherein the sensor device further comprises an acoustic transducer pad comprising a hydrogel that couples to at least some of the acoustic transducer elements of the ultrasound sensor assembly and is configured to provide a contour and cushion against the heart or the blood vessel.
Example B56 includes the sensor device of any of examples B51-B60, wherein the sensor device is deployable within at least one layer of the pericardium of the heart, or wherein the sensor device is deployable on an external layer of the pericardium of the heart.
Example B57 includes the sensor device of any of examples B51-B60, wherein the first arm and the second arm of the clip band are positioned on opposing portions of a left atrium of the heart, and wherein the received acoustic signals are indicative of the blood flow at a mitral valve of the heart.
Example B58 includes the sensor device of any of examples B51-B60, wherein the first arm and the second arm of the clip band are positioned on opposing portions of a right atrium of the heart, and wherein the received acoustic signals are indicative of the blood flow at a tricuspid valve of the heart.
Example B59 includes the sensor device of any of examples B51-B60, the first arm and the second arm of the clip band are positioned on a superior vena cava or an inferior vena cava proximate the heart, and wherein the received acoustic signals are indicative of the blood flow at the superior vena cava or the inferior vena cava.
Example B60 includes the sensor device of any of examples B51-B59, wherein the first arm and the second arm of the clip band are positioned on a pulmonary artery or pulmonary vein proximate the heart, and wherein the received acoustic signals are indicative of the blood flow at the pulmonary artery or the pulmonary vein.

In some embodiments in accordance with the present technology (example B61), a system for in vivo monitoring of fluid flow in an anatomic structure includes an in vivo sensor device operable to be deployed in a body of a patient user and attached to the anatomic structure, and a data processing system in data communication with the in vivo sensor device. The in vivo sensor device includes a linkage assembly comprising a first arm configured to attach to a first portion of the anatomic structure and a second arm configured to attach to a second portion of the anatomic structure opposite to the first portion; a connection apparatus coupled to each of the first arm and the second arm; an ultrasound sensor assembly comprising a plurality of acoustic transducer elements coupled to the linkage assembly, the plurality of acoustic transducer elements including a first acoustic transducer element that is configured to transmit an acoustic signal to propagate through the anatomic structure and a second acoustic transducer element and a third acoustic transducer element that are configured to receive acoustic signals that have propagated through the anatomic structure and are indicative of a fluid flow parameter of a biological fluid in the anatomic structure; and an electronics unit housed in the connection apparatus and in electrical communication with the plurality of acoustic transducer elements of the ultrasound sensor assembly, the electronics unit configured to process electrical signals associated with the received acoustic signals as data and wirelessly transmit the data to an external processor. The data processing system includes a processor and a memory and is configured to receive the data from the in vivo sensor device and process the received data to determine the fluid flow parameter associated with the biological fluid in the anatomic structure.
Example B62 includes the sensor device of any of examples B61-B72, wherein the in vivo sensor device includes one or more features associated with the sensor device recited in any of examples B1-B50 and/or any of examples B51-B60.
Example B63 includes the sensor device of any of examples B61-B72, further comprising a remote in vivo intermediary device comprising a power supply, a wireless communication unit that includes at least one of a transmitter or a transceiver, and a data processing unit that includes a data processor and a data memory, wherein the remote in vivo intermediary device is in wireless or wired communication with the in vivo sensor device and is located in another location of the body different than and at a distance from the anatomic structure.
Example B64 includes the sensor device of example B63 or any of examples B61-B72, wherein the in vivo sensor device is in wired communication with the remote in vivo intermediary device via at least one of a cable, wire, or cord that couples to a port of the connection apparatus of the in vivo sensor device that is in electrical communication with the electronics unit of the in vivo sensor device, and wherein the remote in vivo intermediary device is operable to supply electrical energy stored in the power supply to the electronics unit of the in vivo sensor device.
Example B65 includes the sensor device of example B63 or any of examples B61-B72, wherein the remote in vivo intermediary device is operable to interface the electronics unit of the in vivo sensor device to the data processing unit disposed in the remote in vivo intermediary device to process the data and to determine the fluid flow parameter associated with the biological fluid in the anatomic structure.
Example B66 includes the sensor device of example B63 or any of examples B61-B72, wherein the remote in vivo intermediary device is operable to interface the electronics unit of the in vivo sensor device to the wireless communications unit disposed in the remote in vivo intermediary device to wirelessly transmit the data directly or indirectly to the data processing system.
Example B67 includes the sensor device of example B63 or any of examples B61-B72, wherein the other location where the remote device is located is at least 2 cm from the anatomic structure.
Example B68 includes the sensor device of example B63 or any of examples B61-B72, wherein the other location includes a pleural cavity, a cavity in an abdominal space, a subcutaneous space, or an outer-body location.
Example B69 includes the sensor device of any of examples B61-B72, wherein the data processing system includes a server computer comprising the processor and the memory and one or more databases in data communication with the server computer, wherein the data processing system is configured to remotely monitor data associated with the patient user obtained by the sensor device.
Example B70 includes the sensor device of any of examples B61-B72, further comprising a receiver device, comprising a data processor and a data memory, operable to (i) receive a wireless transmission carrying data indicative of the electrical signal acquired from the sensor device and (ii) transmit the data to the data processing system.
Example B71 includes the sensor device of example B64 or any of examples B61-B72, wherein the receiver device is configured to store the data in the memory of the receiver device, and/or wherein the receiver device is in communication with the data processing system via a network of computers in communication with each other and accessible through the Internet.
Example B72 includes the sensor device of any of examples B61-B71, further comprising a remote client computing device, comprising a data processor and a data memory, in data communication with the data processing system and configured to receive processed data that is selected, filtered, and/or formatted by the data processing system.

In some embodiments in accordance with the present technology (example B73), a sensor device for in vivo monitoring of a cardiac function includes a linkage assembly comprising a first arm configured to attach to a first external portion of a heart, and an optional second arm that may be configured to attach to a second external portion of the heart; a housing coupled to the linkage assembly; a sensor assembly coupled to the first arm and configured to detect a functional parameter of the heart; and an electronics unit disposed in the housing and in electrical communication with the sensor assembly, the electronics unit configured to process electrical signals associated with the detected functional parameter as data and wirelessly transmit the data to an external processor.
Example B74 includes the sensor device of any of examples B73-B76, wherein the sensor device is deployable within at least one layer of the pericardium of the heart, or wherein the sensor device is deployable on an external layer of the pericardium of the heart.
Example B75 includes the sensor device of any of examples B73-B76, wherein the sensor assembly comprises at least one of an accelerometer, a gyroscope, a magnetometer, an acoustic transducer, a temperature sensor, an analyte sensor, or a pH sensor.
Example B76 includes the sensor device of any of examples B73-B76, wherein the sensor device includes one or more features associated with the sensor device recited in any of examples B1-B50 and/or any of examples B51-B60.

In some embodiments in accordance with the present technology (example C1), an implantable medical device (IMD) includes an acoustic sensor.
Example C2 includes the IMD of example C1 or any of examples C1-C25, where the IMD comprises electronic components and a housing that encases the electronic components.
Example C3 includes the IMD of example C1 or any of examples C1-C25, where the housing provides a hermetic seal around the electronic components.
Example C4 includes the IMD of example C1 or any of examples C1-C25, where the housing is biocompatible and is optionally formed from materials comprising organic polymer.
Example C5 includes the IMD of example C2 or any of examples C1-C25, where the acoustic sensor is a microphone.
Example C6 includes the IMD of example C5 or any of examples C1-C25, where the microphone comprises a flexible membrane that undergoes a deflection in response to an acoustic wave contacting an outer surface of the flexible membrane, where the membrane is a component of the housing, and the microphone further comprises an electronic component responsive to the deflection.
Example C7 includes the IMD of example C6 or any of examples C1-C25, where the electronic component responsive to the deflection comprises a strain gauge.
Example C8 includes the IMD of example C6 or any of examples C1-C25, where the electronic component responsive to the deflection comprises a piezoelectric element.
Example C9 includes the IMD of example C6 or any of examples C1-C25, where the electronic component responsive to the deflection comprises a fixed position electrode that does not change location relative to the flexible membrane when the flexible membrane undergoes a deflection in response to the acoustic wave contacting the outer surface of the flexible membrane.
Example C10 includes the IMD of example C6 or any of examples C1-C25, where the electronic component responsive to the deflection comprises an electret condenser element.
Example C11 includes the IMD of example C6 or any of examples C1-C25, where the electronic component responsive to the deflection comprises a capacitive electrode.
Example C12 includes the IMD of example C6 or any of examples C1-C25, where the electronic component responsive to the deflection is affixed to an inner surface of the flexible membrane.
Example C13 includes the IMD of example C2 or any of examples C1-C25, where the acoustic sensor is an accelerometer, referred to as an acoustic accelerometer.
Example C14 includes the IMD of example C13 or any of examples C1-C25, where the acoustic accelerometer senses an acceleration frequency in a range selected from 4000 Hz to 5000 Hz, or 3000 Hz to 4000 Hz, or 2000 Hz to 3000 Hz.
Example C15 includes the IMD of example C13 or any of examples C1-C25, where the acoustic accelerometer samples data at a sampling frequency selected from 800 to 20,000 samples per second, e.g., 1,400 to 14,000 samples per second.
Example C16 includes the IMD of example C13 or any of examples C1-C25, where the acoustic accelerometer operates at a noise level of between 200 micro-g/rtHz and 0.5 micro-g/rtHz.
Example C17 includes the IMD of example C13 or any of examples C1-C25, where the acoustic accelerometer operates at a high precision between 10 micro-g and 1 micro-g.
Example C18 includes the IMD of example C13 or any of examples C1-C25, where the acoustic accelerometer is a multi-axial accelerometer.
Example C19 includes the IMD of example C13 or any of examples C1-C25, where a mass density of a combination of the housing and the electronic components encased by the housing is less than a mass density of 1000 kg/m³.
Example C20 includes the IMD of example C1 or any of examples C1-C25, where the IMD further comprises a motion sensor to enable the IMD to gather data indicative of position and movement of the host.
Example C21 includes the IMD of example C1 or any of examples C1-C25, where the IMD further comprises an ECG sensor to enable the IMD to gather data from which an electrocardiogram indicative of the health of the host can be prepared.
Example C22 includes the IMD of example C1 or any of examples C1-C25, where the IMD further comprises an ultrasonic sensor assembly comprising one or more ultrasonic transducer elements.
Example C23 includes the IMD of example C1 or any of examples C1-C25, where the IMD further comprises a motion sensor to enable the IMD to gather data indicative of position and movement of the host, and an ECG sensor to enable the IMD to gather data from which an electrocardiogram indicative of the health of the host can be prepared.
Example C24 includes the IMD of example C1 or any of examples C1-C25, where the IMD further comprises a motion sensor to enable the IMD to gather data indicative of position and movement of the host, and an ultrasonic sensor assembly comprising one or more ultrasonic transducer elements.
Example C25 includes the IMD of example C1 or any of examples C1-C25, where the IMD further comprises a motion sensor to enable the IMD to gather data indicative of position and movement of the host; an ECG sensor to enable the IMD to gather data from which an electrocardiogram indicative of the health of the host can be prepared; and an ultrasonic sensor assembly comprising one or more ultrasonic transducer elements.

In some embodiments in accordance with the present technology (example C26), a method of operating an implantable medical device (IMD), where the IMD comprises, an accelerometer contained in a hermetic environment of the IMD, where the accelerometer is caused to operate at one or more of the following conditions: (i) the accelerometer senses an acceleration frequency in a range selected from 4000 Hz to 5000 Hz, or 3000 Hz to 4000 Hz, or 2000 Hz to 3000 Hz; (ii) the accelerometer samples data at a sampling frequency selected from 800 to 20,000 samples per second, e.g., 1,400 to 14,000 samples per second; (iii) the accelerometer operates at a noise level of between 200 micro-g/rtHz and 0.5 micro-g/rtHz; and/or (iv) the accelerometer operates at a high precision between 10 micro-g and 1 micro-g.

In some embodiments in accordance with the present technology (example C27), a method of monitoring a medical condition in a host includes operating an IMD according to any of examples C1-C25 or example C26 in the host and obtaining data from acoustic waves generated by the host that are indicative of the presence and quality of the medical condition.
Example C28 includes the method of example C27 or example C26, where the medical condition includes: heart disease; a valvular disease selected from stenosis, incontinence and myxomatous, where the valvular disease is associated with a valve selected from tricuspid, pulmonary, pulmonic, mitral and aortic, e.g., the medical condition may be myxomatous mitral valve disease; congestive heart failure; atrial fibrillation; coronary artery disease; emphysema; pulmonary embolism; pulmonary disease; chronic obstructive pulmonary disease (COPD); and/or asthma.

In some embodiments in accordance with the present technology (example C29), a method for monitoring a physical condition of a host with an implantable medical device (IMD), where the IMD comprises an acoustic sensor, or, optionally, an IMD of any of examples C1-C25, where the method includes one or more of: manufacturing the IMD; performing quality control on the IMD; packaging the IMD for shipment or storage; making the IMD available to a surgeon; providing instructions for use of the IMD; identifying a host having a medical condition in need of monitoring; implanting the IMD into a host, e.g., into a coelom of a host, into a ventral cavity of the host, into a dorsal cavity of a host, into a thoracic cavity of a host, into an upper ventral cavity of the host, into a lower ventral cavity of the host, into an upper dorsal cavity of the host, into a lower dorsal cavity of the host, into a pleural cavity of the host, into a pericardial cavity of the host, into an abdominopelvic cavity of the host, into an abdominal cavity of a host; into a pelvis of the host, into a spinal cavity of the host; assisting a surgeon in implanting the IMD into a host; operating robotics to assist a surgeon in implanting the IMD into a host; transmitting a wireless signal to the implanted IMD and thereby activating the implanted IMD; sensing acoustic signals generated by the host with the acoustic sensor of the implanted IMD, where optionally the acoustic signals are generated by one or more of the heart of the host, the blood flow of the host, the respiration of the host; storing sensed acoustic signals or a derivative thereof in a memory located within the IMD to provide stored data, where optionally the stored data is stored in the memory for at least 1 hour, or at least 6 hours, or at least 12 hours, or at least 18 hours, or at least 24 hours, or at least 48 hours; transmitting the stored data from the implanted IMD to a receiving device located outside of the host, to provide received data that is stored on a memory of the receiving device; analyzing the received data to generate health information that is informative as to the physical condition of the host; providing the health information to a health care provider (HCP); and/or evaluating the health information as a component of developing a health care plan for the host.
Example C30 includes the method of example C29, where the medical condition in need of monitoring includes: heart disease; a valvular disease selected from stenosis, incontinence and myxomatous, where the valvular disease is associated with a valve selected from tricuspid, pulmonary, pulmonic, mitral and aortic, e.g., the medical condition may be myxomatous mitral valve disease; congestive heart failure; atrial fibrillation; coronary artery disease; emphysema; pulmonary embolism; pulmonary disease; chronic obstructive pulmonary disease (COPD); and/or asthma.

In some embodiments in accordance with the present technology (example D1), an implantable medical device includes an in vivo fluid flow sensor; and an in vivo acoustic sensor.
Example D2 includes the device of example D1 or any of examples D1-D20, wherein the in vivo fluid flow sensor is configured to transmit an ultrasound signal to propagate through an anatomic structure and to detect ultrasound signals that have propagated through the anatomic structure and are indicative of a fluid flow of a biological fluid in the anatomic structure.
Example D3 includes the device of example D2 or any of examples D1-D20, wherein the in vivo fluid flow sensor comprises: a linkage assembly comprising a first arm configured to attach to a first portion of the anatomic structure and a second arm configured to attach to a second portion of the anatomic structure opposite to the first portion, an ultrasound sensor assembly comprising a plurality of ultrasound transducer elements coupled to the linkage assembly.
Example D4 includes the device of example D3 or any of examples D1-D20, wherein the plurality of ultrasound transducer elements include a first ultrasound transducer element that is configured to transmit the ultrasound signal to propagate through the anatomic structure and a second ultrasound transducer element and a third ultrasound transducer element that are configured to receive the ultrasound signals that have propagated through the anatomic structure and are indicative of a fluid flow parameter of the biological fluid in the anatomic structure.
Example D5 includes the device of example D1 or any of examples D1-D20, wherein the in vivo acoustic sensor is configured to detect an acoustic signal emanating from an internal body structure.
Example D6 includes the device of example D5 or any of examples D1-D20, wherein the in vivo acoustic sensor comprises: a hermetically sealed housing, and a transducer element configured to receive the acoustic signal that emanates from the internal body structure such that the transducer element converts energy of the received acoustic signal to an electrical signal indicative of a physiological function by the internal body structure.
Example D7 includes the device of example D1 or any of examples D1-D20, wherein the in vivo acoustic sensor includes a displacement-mediated acoustic sensor to measure a change in conformation of a transducer element caused by the acoustic signal on the transducer element.
Example D8 includes the device of example D7 or any of examples D1-D20, wherein the in vivo acoustic sensor includes a microphone.
Example D9 includes the device of example D7 or any of examples D1-D20, wherein the in vivo acoustic sensor includes an accelerometer.
Example D10 includes the device of example D7 or any of examples D1-D20, wherein the in vivo acoustic sensor includes a strain gauge.
Example D11 includes the device of example D7 or any of examples D1-D20, wherein the in vivo acoustic sensor includes a pressure sensor.
Example D12 includes the device of example D1 or any of examples D1-D20, wherein the in vivo acoustic sensor includes a stress-mediated acoustic sensor operable to measure stress caused by the acoustic signal applied on a transducer element.
Example D13 includes the device of example D12 or any of examples D1-D20, wherein the in vivo acoustic sensor includes a unimorph piezoelectric sensor device.
Example D14 includes the device of example D1 or any of examples D1-D20, wherein the acoustic signal includes a transmission of mechanical energy that propagates in an in vivo medium including one or more of a gas, liquid, or solid.
Example D15 includes the device of example D1 or any of examples D1-D20, wherein the implantable medical device includes an electronics unit in electrical communication with in vivo fluid flow sensor and with the in vivo acoustic sensor, wherein the electronics unit is in a hermetically sealed casing.
Example D16 includes the device of example D15 or any of examples D1-D20, wherein the electronics unit comprises a signal processing unit and a wireless communications unit configured to process electrical signals associated with the detected ultrasound signals and/or the detected acoustic signals as data and wirelessly transmit the data to an external processor.
Example D17 includes the device of example D15 or any of examples D1-D20, wherein the electronics unit comprises a power supply.
Example D18 includes the device of example D15 or any of examples D1-D20, wherein the signal processing unit includes a signal conditioning circuit configured to process the electrical signals associated with the received ultrasound signals by one or more of amplifying the electrical signals, filtering the electrical signals, or converting the electrical signals from analog to digital.
Example D19 includes the device of example D18 or any of examples D1-D20, wherein the electronics unit comprises a data processing unit in communication with the signal conditioning circuit, the data processing unit comprising a processor and a memory and configured to process the amplified, filtered, or converted electrical signals as biomedical data.
Example D20 includes the device of example D15 or any of examples D1-D20, wherein the electronics unit comprises a printed circuit board (PCB) having a substrate and electrical interconnections disposed on the substrate, wherein the electrical interconnections are coupled to a plurality of electrical interconnection wires that span between the sensors of the in vivo fluid flow sensor and the in vivo acoustic sensor.

In some embodiments in accordance with the present technology (example D21), an implantable medical device for in vivo monitoring of an anatomic structure from within a host's body includes (i) an in vivo fluid flow sensor, which comprises a linkage assembly comprising a first arm configured to attach to a first portion of the anatomic structure and a second arm configured to attach to a second portion of the anatomic structure opposite to the first portion, a connection apparatus coupled to each of the first arm and the second arm, an ultrasound sensor assembly comprising a plurality of ultrasound transducer elements coupled to the linkage assembly, the plurality of ultrasound transducer elements including a first ultrasound transducer element that is configured to transmit an ultrasound signal to propagate through the anatomic structure and a second ultrasound transducer element and a third ultrasound transducer element that are configured to receive ultrasound signals that have propagated through the anatomic structure and are indicative of a fluid flow parameter of a biological fluid in the anatomic structure; (ii) an in vivo acoustic sensor, which comprises a hermetically sealed housing, and a transducer element configured to receive an acoustic signal that emanates from a source within the host's body such that the transducer element converts energy of the received acoustic signal to an electrical signal indicative of a physiological function by the source within the host's body; and (iii) an electronics unit at least partially housed in the connection apparatus of the in vivo fluid flow sensor and/or at least partially housed in the hermetically sealed housing of the in vivo fluid flow sensor, wherein the electronics unit is in electrical communication with the plurality of ultrasound transducer elements of the ultrasound sensor assembly and in electrical communication with the transducer element of the in vivo acoustic sensor, the electronics unit configured to process electrical signals associated with the received ultrasound signals and the received acoustic signal as data and wirelessly transmit the data to an external processor.
Example D22 includes the device of example D21 or any of examples D21-D86, wherein the in vivo acoustic sensor includes a stress-mediated acoustic sensor.
Example D23 includes the device of example D22 or any of examples D21-D86, wherein the transducer element includes a piezoelectric material.
Example D24 includes the device of example D23 or any of examples D21-D86, wherein the piezoelectric material includes one or more of lead zirconate titanate (PZT), lead lanthanum zirconate titanate (PLZT), zinc oxide (ZnO), quartz, polyvinylidene fluoride or polyvinylidene difluoride (PVDF) aluminum nitride (AlN), scandium aluminum nitride (ScAlN), barium titanate (BaTiO₃), lead titanate (PbTiO₃), potassium niobate (KNbO₃), lithium niobate (LiNbO₃), lithium tantalate (LiTaO₃), and/or sodium tungstate (Na₂WO₄).
Example D25 includes the device of example D23 or any of examples D21-D86, wherein the stress-mediated acoustic sensor includes a unimorph piezoelectric sensor, comprising: a biocompatible, electrically conductive, non-piezoelectric material coupled to a first side of the piezoelectric material and configured in the hermetically sealed housing to face outward of the in vivo acoustic sensor, the biocompatible, electrically conductive, non-piezoelectric material having a Young's modulus within 2×-3× of the Young's modulus of the piezoelectric material, and an electrically conductive, non-piezoelectric material coupled to a second side of the piezoelectric material opposite the first side, wherein the biocompatible, electrically conductive, non-piezoelectric material is configured to receive an applied force caused from the acoustic signal emanated from the source within the host's body, such that stress caused by the applied force on the biocompatible, electrically conductive, non-piezoelectric material is transferred through and into the piezoelectric material to transduce stress into electrical energy corresponding to the electrical signal captured at the electrically conductive, non-piezoelectric material.
Example D26 includes the device of example D25 or any of examples D21-D86, wherein the biocompatible, electrically conductive, non-piezoelectric material includes one or more of titanium (Ti), a biocompatible stainless-steel, a cobalt-chromium alloy, nitinol, or a combination thereof.
Example D27 includes the device of example D23 or any of examples D21-D86, wherein the stress-mediated acoustic sensor includes a unimorph piezoelectric sensor, comprising: a biocompatible, electrically insulative, non-piezoelectric material configured in the hermetically sealed housing to face outward of the in vivo acoustic sensor, the biocompatible, electrically insulative, non-piezoelectric material having a Young's modulus within 2×-3× of the Young's modulus of the piezoelectric material, a first electrically conductive, non-piezoelectric material coupled to a first side of the piezoelectric material, and an electrically conductive, non-piezoelectric material coupled to a second side of the piezoelectric material opposite the first side, wherein the biocompatible, electrically insulative, non-piezoelectric material is configured to receive an applied force caused from the acoustic signal emanated from the source within the host's body, such that stress caused by the applied force on the biocompatible, electrically insulative, non-piezoelectric material is transferred through and into the piezoelectric material to transduce stress into electrical energy corresponding to the electrical signal captured at the first and second electrically conductive, non-piezoelectric materials.
Example D28 includes the device of example D27 or any of examples D21-D86, wherein the biocompatible, electrically insulative, non-piezoelectric material includes a high-purity ceramic.
Example D29 includes the device of example D22 or any of examples D21-D86, wherein the stress-mediated acoustic sensor does not involve a displacement of the transducing element.
Example D30 includes the device of example D21 or any of examples D21-D86, wherein the in vivo acoustic sensor further comprises a casing structure that provides a firm, inflexible material and is configured to secure and/or position the transducer element in the hermetically sealed housing.
Example D31 includes the device of example D30 or any of examples D21-D86, wherein the casing structure part of the hermetically sealed housing.
Example D32 includes the device of example D30 or example D31 or any of examples D21-D86, wherein the casing structure includes titanium (Ti).
Example D33 includes the device of example D21 or any of examples D21-D86, wherein the in vivo acoustic sensor includes a displacement-mediated acoustic sensor.
Example D34 includes the device of example D33 or any of examples D21-D86, wherein the transducer element includes a microphone.
Example D35 includes the device of example D34 or any of examples D21-D86, wherein the microphone comprises a membrane that undergoes a deflection in response to an acoustic wave contacting an outer surface of the membrane, where the membrane is coupled to or is a component of the hermetically sealed housing, and wherein the microphone further comprises an electronic component responsive to the deflection.
Example D36 includes the device of example D35 or any of examples D21-D86, wherein the electronic component responsive to the deflection includes at least one of a strain gauge, a piezoelectric element, a condenser element, a capacitive electrode, or a fixed position electrode that does not change location relative to the membrane when the membrane undergoes a deflection in response to the acoustic wave contacting the outer surface of the flexible membrane.
Example D37 includes the device of example D33 or any of examples D21-D86, wherein the transducer element includes an accelerometer.
Example D38 includes the device of example D21 or any of examples D21-D86, wherein the ultrasound sensor assembly includes a first ultrasound sensor assembly disposed on the first arm of the linkage assembly and a second ultrasound sensor assembly disposed on the second arm of the linkage assembly.
Example D39 includes the device of example D38 or any of examples D21-D86, wherein the first ultrasound sensor assembly includes the first ultrasound transducer element configured to transmit the ultrasound signal that is positioned on a distal region of the first arm to interface with the first portion of the anatomic structure, wherein the second ultrasound sensor assembly includes the second and third ultrasound transducer elements configured to receive the ultrasound signals that are positioned on a distal region of the second arm to interface with the second portion of the anatomic structure, and wherein a first center point (C_{Tx}) of the first ultrasound transducer element is aligned with a second center point (Cd_{Rx}) located between a distance (d) separating centers of the second ultrasound transducer element and the third ultrasound transducer element.
Example D40 includes the device of example D38 or any of examples D21-D86, wherein the first ultrasound sensor assembly includes the first ultrasound transducer element configured to transmit the ultrasound signal that is positioned on a distal region of the first arm to interface with the first portion of the anatomic structure, wherein the second ultrasound sensor assembly includes the second and third ultrasound transducer elements configured to receive the ultrasound signals that are positioned on a distal region of the second arm to interface with the second portion of the anatomic structure, wherein the first ultrasound sensor assembly further includes a fourth ultrasound transducer element and a fifth ultrasound transducer element positioned on the distal region of the second arm to interface with the second portion of the anatomic structure and configured to receive a first set of ultrasound signals associated with transmission of the ultrasound signal by the first ultrasound transducer element, wherein the second ultrasound sensor assembly includes a sixth ultrasound transducer element positioned on the distal region of the first arm to interface with the first portion of the anatomic structure and configured to transmit a second ultrasound signal associated with the ultrasound signals received at the second and third ultrasound transducer elements, and wherein a first center point (C_{Tx1}) of the first ultrasound transducer element is aligned with a second center point (Cd_{Rx1}) located between a first distance (d1) separating centers of the fourth ultrasound transducer element and the fifth ultrasound transducer element, and wherein a third center point (C_{Tx2}) of the sixth ultrasound transducer element is aligned with a fourth center point (Cd_{Rx2}) located between a second distance (d2) separating centers of the second ultrasound transducer element and the third ultrasound transducer element.
Example D41 includes the device of example D21 or any of examples D21-D86, wherein the plurality of ultrasound transducer elements of the ultrasound sensor assembly is disposed on the first arm of the linkage assembly, and wherein the in vivo fluid flow sensor further comprises a reflector disposed on the second arm of the linkage assembly.
Example D42 includes the device of example D21 or any of examples D21-D86, wherein at least one of the first ultrasound transducer element, the second transducer element, or the third ultrasound transducer element is configured to have a size dimension between 1 mm to 4 mm.
Example D43 includes the device of example D21 or any of examples D21-D86, wherein the ultrasound sensor assembly further comprises a substrate connected to an interior-facing surface of at least one of the first arm or the second arm of the linkage assembly and that couples at least one of the plurality of ultrasound transducer elements.
Example D44 includes the device of example D43 or any of examples D21-D86, wherein the substrate is configured to provide a heat sync for management of thermal generation by the at least one ultrasound transducer element.
Example D45 includes the device of example D21 or any of examples D21-D86, wherein at least one of the plurality of ultrasound transducer elements includes a piezoelectric micromachined ultrasonic transducer (PMUT).
Example D46 includes the device of example D21 or any of examples D21-D86, wherein the in vivo fluid flow sensor further comprises an ultrasound transducer pad that couples to at least some of the ultrasound transducer elements of the ultrasound sensor assembly and is configured to provide a contour and cushion against the anatomic structure.
Example D47 includes the device of example D46 or any of examples D21-D86, wherein the ultrasound transducer pad includes a hydrogel.
Example D48 includes the device of example D21 or any of examples D21-D86, wherein the linkage assembly is operable to secure the in vivo fluid flow sensor to the anatomic structure with pliability for the in vivo fluid flow sensor to be stable in its placement with respect to the anatomic structure while withstanding continuous movements of the anatomic structure to which its attached.
Example D49 includes the device of example D48 or any of examples D21-D86, wherein the linkage assembly includes a clip band operable to provide a compression force by each of the first arm and the second arm of the clip band to facilitate securement of the in vivo fluid flow sensor to the first portion and the second portion of the anatomic structure and to absorb forces applied onto the first arm and the second arm of the clip band by the anatomic structure due to the continuous movements of the anatomic structure.
Example D50 includes the device of example D49 or any of examples D21-D86, wherein the clip band comprises a composite material having a polymer component that is flexible and a shape-stiffener component that is pre-shaped and is capable of undergoing a shape change.
Example D51 includes the device of example D50 or any of examples D21-D86, wherein the shape-stiffener component of the composite material comprises one or more of Nitinol, gold, platinum, or iridium, which is encased in the polymer component of the composite material that comprises one or more of silicone, polyethylene, polyimide, polyamide, or a blend thereof.
Example D52 includes the device of example D51 or any of examples D21-D86, wherein the connection apparatus is operable to secure the linkage assembly to the anatomic structure with mechanical resilience for the in vivo fluid flow sensor to be stable in its placement with respect to the anatomic structure while withstanding continuous movements of the anatomic structure to which its attached.
Example D53 includes the device of example D52 or any of examples D21-D86, wherein the connection apparatus includes a spring operable to provide a compression force upon each of the first arm and the second arm of the linkage assembly to facilitate securement of the in vivo fluid flow sensor to the first portion and the second portion of the anatomic structure and to absorb forces applied onto the first arm and the second arm of the linkage assembly by the anatomic structure due to the continuous movements of the anatomic structure.
Example D54 includes the device of example D21 or any of examples D21-D86, wherein the electronics unit comprises a power supply and a wireless communications unit including a wireless transmitter or wireless transceiver.
Example D55 includes the device of example D54 or any of examples D21-D86, wherein the power supply includes at least one of a battery or a fuel cell.
Example D56 includes the device of example D54 or any of examples D21-D86, wherein the electronics unit comprises a signal conditioning unit in communication with the plurality of ultrasound transducer elements of the ultrasound sensor assembly via one or more electrical interface components, the signal conditioning unit comprising an electrical circuit configured to process the electrical signals associated with the received ultrasound signals by one or more of amplifying the electrical signals, filtering the electrical signals, or converting the electrical signals from analog to digital.
Example D57 includes the device of example D56 or any of examples D21-D86, wherein the electronics unit comprises a data processing unit in communication with the signal conditioning unit, the data processing unit comprising a processor and a memory and configured to process the amplified, filtered, or converted electrical signals as the data representative of the fluid flow parameter of the biological fluid.
Example D58 includes the device of example D54 or any of examples D21-D86, wherein the electronics unit comprises a data processing unit in communication with the wireless communications unit, the data processing unit comprising a processor and a memory and configured to process the electrical signals as the data representative of the fluid flow parameter of the biological fluid.
Example D59 includes the device of example D54 or any of examples D21-D86, wherein the electronics unit comprises a printed circuit board (PCB) having a substrate and electrical interconnections disposed on the substrate, wherein the electrical interconnections are coupled to a plurality of electrical interconnection wires that span between the plurality of ultrasound transducer elements of the ultrasound sensor assembly and the PCB of the electronics unit.
Example D60 includes the device of example D54 or any of examples D21-D86, wherein the electronics unit comprises a casing that encompasses the electronics unit to protect the electronics unit from exposure to a body fluid when the implantable medical device is inserted and deployed in vivo.
Example D61 includes the device of example D60 or any of examples D21-D86, wherein the casing includes one or both of flat sides or curved sides to provide a form factor of the implantable medical device, including at least one of rectangular, a cylindrical, a conical, an elliptical, a pyramidal, a trapezoidal, or a non-uniform shape.
Example D62 includes the device of example D60 or any of examples D21-D86, wherein the casing includes is coupled to the linkage assembly on an inward-facing surface of the linkage assembly that faces toward the anatomic structure, or wherein the casing includes is coupled to the linkage assembly on an outward-facing surface of the linkage assembly that faces away from the anatomic structure.
Example D63 includes the device of example D60 or any of examples D21-D86, wherein the PCB of the electronics unit is hermetically sealed within the casing by a non-permeable material that covers the PCB to provide an electrical shield from the body fluid.
Example D64 includes the device of example D63 or any of examples D21-D86, wherein the non-permeable material includes at least one of a parylene, a urethane, or a Teflon material.
Example D65 includes the device of example D21 or any of examples D21-D86, further comprising: a secondary sensor coupled to at least one of the in vivo fluid flow sensor or the in vivo acoustic sensor and in communication with the electronics unit, the secondary sensor operable to measure one or more of a biological parameter, a physiological parameter, an electrophysiological parameter, or a physical parameter of the body within which the device is deployed.
Example D66 includes the device of example D65 or any of examples D21-D86, wherein the secondary sensor includes an analyte sensor configured to detect an analyte of the biological fluid in the anatomic structure or of a body fluid in a region where the implantable medical device is deployed.
Example D67 includes the device of example D65 or any of examples D21-D86, wherein the secondary sensor includes a pH sensor configured to detect a pH level of the biological fluid in the anatomic structure or of a body fluid in a region where the implantable medical device is deployed.
Example D68 includes the device of example D65 or any of examples D21-D86, wherein the secondary sensor includes a temperature sensor configured to detect temperature of the biological fluid in the anatomic structure or of a body fluid in a region where the implantable medical device is deployed.
Example D69 includes the device of example D65 or any of examples D21-D86, wherein the secondary sensor includes an inertial measurement unit (IMU) configured to detect motion of the implantable medical device in multiple degrees of freedom.
Example D70 includes the device of example D21 or any of examples D21-D86, further comprising: a secondary attachment component comprising one or more of a suture, a prong, a screw, a barb, an adhesive, or a grasping mechanism disposed on at least one portion of the linkage assembly and configured to secure the to the ultrasound sensor assembly via the linkage assembly to the anatomic structure.
Example D71 includes the device of example D21 or any of examples D21-D86, wherein the linkage assembly is capable of changing shape from a first shape conformation, where the first arm and the second arm of the linkage assembly spread outward from a centerline through at least one of the linkage assembly or the connection apparatus, to be insertable into a body of a patient, to a second shape conformation, where the first arm and the second arm of the linkage assembly span inward toward the centerline through at least one of the linkage assembly or the connection apparatus, to be attachable to the anatomic structure for operation of the in vivo fluid flow sensor.
Example D72 includes the device of example D21 or any of examples D21-D86, further comprising: a second linkage assembly comprising a third arm configured to attach to a third portion of the anatomic structure and a fourth arm configured to attach to a fourth portion of the anatomic structure opposite to the third portion, wherein the connection apparatus is coupled to each of the third arm and the fourth arm, and wherein the ultrasound sensor assembly comprises an additional set of the plurality of ultrasound transducer elements coupled to the second linkage assembly and configured to transmit a second ultrasound signal to propagate through the anatomic structure across the third portion and the fourth portion and to receive a second set of ultrasound signals that have propagated through the anatomic structure across the third portion and the fourth portion and are indicative of a second fluid flow parameter of the biological fluid in the anatomic structure.
Example D73 includes the device of example D21 or any of examples D21-D86, wherein the connection apparatus includes a port in electrical communication with the electronics unit housed in the connection apparatus and operable to electrically couple to a remote power supply housed in a remote device via at least one of a cable, wire, or cord, wherein the remote device is located in another location different than and at a distance from the anatomic structure.
Example D74 includes the device of example D73 or any of examples D21-D86, wherein the port is in data communication with the electronics unit housed in the connection apparatus and operable to interface the electronics unit to a data processing unit disposed in the remote device via the at least one of the cable, wire, or cord to output the data from the electronics unit of the sensor device to the data processing unit of the remote device through the port via the at least one of the cable, wire, or cord.
Example D75 includes the device of example D74 or any of examples D21-D86, wherein the implantable medical device is configured to wirelessly transmit the data by a secondary transmission device comprising a wireless transmitter or transceiver to the external processor.
Example D76 includes the device of example D73 or any of examples D21-D86, wherein the other location where the remote device is located is at least 2 cm from the anatomic structure.
Example D77 includes the device of example D73 or any of examples D21-D86, wherein the other location includes a pleural cavity, a cavity in an abdominal space, a subcutaneous space, or an outer-body location.
Example D78 includes the device of example D21 or any of examples D21-D86, wherein the external processor that is able to receive the data to be wirelessly transmitted by the electronics unit of the sensor device is located outside of the body of a patient user to which the sensor device is implantable and deployable at the anatomic structure.
Example D79 includes the device of examples D21-D78 or any of examples D21-D86, wherein the fluid flow parameter of the biological fluid in the anatomic structure includes at least one of a flow rate, or an amount of pressure difference between ends of a flow path in the anatomic structure.
Example D80 includes the device of example D79 or any of examples D21-D86, wherein the device is operable to detect a size dimension of the anatomic structure or a predictive area or volume of the anatomic structure based on a baseline measurement of the flow rate and changes in the flow rate over time.
Example D81 includes the device of examples D21-D80 or any of examples D21-D86, wherein the anatomic structure is a heart, and the biological fluid is blood.
Example D82 includes the device of example D81 or any of examples D21-D86, wherein the in vivo fluid flow sensor is deployable within at least one layer of the pericardium of the heart, or wherein the sensor device is deployable on an external layer of the pericardium of the heart.
Example D83 includes the device of example D81 or any of examples D21-D86, wherein the first arm and the second arm of the linkage assembly are positioned on opposing portions of a left atrium of the heart, and wherein the received ultrasound signals are indicative of blood flow at a mitral valve of the heart.
Example D84 includes the device of example D81 or any of examples D21-D86, wherein the first arm and the second arm of the linkage assembly are positioned on opposing portions of a right atrium of the heart, and wherein the received ultrasound signals are indicative of blood flow at a tricuspid valve of the heart.
Example D85 includes the device of example D81 or any of examples D21-D86, wherein the first arm and the second arm of the linkage assembly are positioned on a superior vena cava or an inferior vena cava proximate the heart, and wherein the received ultrasound signals are indicative of blood flow at the superior vena cava or the inferior vena cava.
Example D86 includes the device of example D81 or any of examples D21-D86, wherein the first arm and the second arm of the linkage assembly are positioned on a pulmonary artery or pulmonary vein proximate the heart, and wherein the received ultrasound signals are indicative of blood flow at the pulmonary artery or the pulmonary vein.

In some embodiments in accordance with the present technology (example D87), a system for in vivo monitoring of fluid flow in an anatomic structure includes the implantable medical device of any of examples D1-D86; and a data processing system, comprising a processor and a memory, in data communication with the implantable medical device and configured to receive the data from the implantable medical device and process the received data to determine a fluid flow parameter associated with a biological fluid in the anatomic structure and/or an acoustic signal parameter associated with a physiological function of an internal body structure.

### CONCLUSION

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” may include the plural forms as well, unless the context clearly indicates otherwise. Additionally, the use of “or” may include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. An implantable medical device, comprising:
an in vivo fluid flow sensor; and
an in vivo acoustic sensor.

2. The device of claim 1, wherein the in vivo fluid flow sensor is configured to transmit an ultrasound signal to propagate through an anatomic structure and to detect ultrasound signals that have propagated through the anatomic structure and are indicative of a fluid flow of a biological fluid in the anatomic structure.

3. The device of claim 2, wherein the in vivo fluid flow sensor comprises:
a linkage assembly comprising a first arm configured to attach to a first portion of the anatomic structure and a second arm configured to attach to a second portion of the anatomic structure opposite to the first portion, and
an ultrasound sensor assembly comprising a plurality of ultrasound transducer elements coupled to the linkage assembly.

4. The device of claim 3, wherein the plurality of ultrasound transducer elements include a first ultrasound transducer element that is configured to transmit the ultrasound signal to propagate through the anatomic structure and a second ultrasound transducer element and a third ultrasound transducer element that are configured to receive the ultrasound signals that have propagated through the anatomic structure and are indicative of a fluid flow parameter of the biological fluid in the anatomic structure.

5. The device of claim 1, wherein the in vivo acoustic sensor is configured to detect an acoustic signal emanating from an internal body structure.

6. The device of claim 5, wherein the in vivo acoustic sensor comprises:
a hermetically sealed housing, and
a transducer element configured to receive the acoustic signal that emanates from the internal body structure such that the transducer element converts energy of the received acoustic signal to an electrical signal indicative of a physiological function by the internal body structure.

7-15. (canceled)

16. The device of claim 1, further comprising an electronics unit in electrical communication with in vivo fluid flow sensor and with the in vivo acoustic sensor, wherein the electronics unit is in a hermetically sealed casing and wherein the electronics unit comprises a signal processing unit and a wireless communications unit configured to process electrical signals associated with the detected ultrasound signals and/or the detected acoustic signals as data and wirelessly transmit the data to an external processor.

17-20. (canceled)

21. An implantable medical device for in vivo monitoring of an anatomic structure from within a host's body, comprising:
an in vivo fluid flow sensor, comprising:
a linkage assembly comprising a first arm configured to attach to a first portion of the anatomic structure and a second arm configured to attach to a second portion of the anatomic structure opposite to the first portion,
a connection apparatus coupled to each of the first arm and the second arm,
an ultrasound sensor assembly comprising a plurality of ultrasound transducer elements coupled to the linkage assembly, the plurality of ultrasound transducer elements including a first ultrasound transducer element that is configured to transmit an ultrasound signal to propagate through the anatomic structure and a second ultrasound transducer element and a third ultrasound transducer element that are configured to receive ultrasound signals that have propagated through the anatomic structure and are indicative of a fluid flow parameter of a biological fluid in the anatomic structure;
an in vivo acoustic sensor, comprising:
a hermetically sealed housing, and
a transducer element configured to receive an acoustic signal that emanates from a source within the host's body such that the transducer element converts energy of the received acoustic signal to an electrical signal indicative of a physiological function by the source within the host's body; and
an electronics unit at least partially housed in the connection apparatus of the in vivo fluid flow sensor and/or at least partially housed in the hermetically sealed housing of the in vivo fluid flow sensor, wherein the electronics unit is in electrical communication with the plurality of ultrasound transducer elements of the ultrasound sensor assembly and in electrical communication with the transducer element of the in vivo acoustic sensor, the electronics unit configured to process electrical signals associated with the received ultrasound signals and the received acoustic signal as data and wirelessly transmit the data to an external processor.

22. The device of claim 21, wherein the in vivo acoustic sensor includes a stress-mediated acoustic sensor.

23. The device of claim 22, wherein the transducer element includes a piezoelectric material.

24. The device of claim 23, wherein the piezoelectric material includes one or more of lead zirconate titanate (PZT), lead lanthanum zirconate titanate (PLZT), zinc oxide (ZnO), quartz, polyvinylidene fluoride or polyvinylidene difluoride (PVDF) aluminum nitride (AlN), scandium aluminum nitride (ScAlN), barium titanate (BaTiO₃), lead titanate (PbTiO₃), potassium niobate (KNbO₃), lithium niobate (LiNbO₃), lithium tantalate (LiTaO₃), and/or sodium tungstate (Na₂WO₄).

25-26. (canceled)

27. The device of claim 23, wherein the stress-mediated acoustic sensor includes a unimorph piezoelectric sensor, comprising:
a biocompatible, electrically insulative, non-piezoelectric material configured in the hermetically sealed housing to face outward of the in vivo acoustic sensor, the biocompatible, electrically insulative, non-piezoelectric material having a Young's modulus within 2×-3× of the Young's modulus of the piezoelectric material,
a first electrically conductive, non-piezoelectric material coupled to a first side of the piezoelectric material, and
an electrically conductive, non-piezoelectric material coupled to a second side of the piezoelectric material opposite the first side,
wherein the biocompatible, electrically insulative, non-piezoelectric material is configured to receive an applied force caused from the acoustic signal emanated from the source within the host's body, such that stress caused by the applied force on the biocompatible, electrically insulative, non-piezoelectric material is transferred through and into the piezoelectric material to transduce stress into electrical energy corresponding to the electrical signal captured at the first and second electrically conductive, non-piezoelectric materials.

28-29. (canceled)

30. The device of claim 21, wherein the in vivo acoustic sensor further comprises a casing structure that provides a firm, inflexible material and is configured to secure and/or position the transducer element in the hermetically sealed housing.

31-43. (canceled)

44. The device of claim 21 wherein the ultrasound sensor assembly further comprises a substrate connected to an interior-facing surface of at least one of the first arm or the second arm of the linkage assembly and that couples at least one of the plurality of ultrasound transducer elements, claim 43, wherein the substrate is configured to provide a heat sync for management of thermal generation by the at least one ultrasound transducer element.

45. (canceled)

46. The device of claim 21, wherein the in vivo fluid flow sensor further comprises an ultrasound transducer pad that couples to at least some of the ultrasound transducer elements of the ultrasound sensor assembly and is configured to provide a contour and cushion against the anatomic structure.

47. The device of claim 46, wherein the ultrasound transducer pad includes a hydrogel.

48-71. (canceled)

72. The device of claim 21, further comprising:
a second linkage assembly comprising a third arm configured to attach to a third portion of the anatomic structure and a fourth arm configured to attach to a fourth portion of the anatomic structure opposite to the third portion, wherein the connection apparatus is coupled to each of the third arm and the fourth arm, and wherein the ultrasound sensor assembly comprises an additional set of the plurality of ultrasound transducer elements coupled to the second linkage assembly and configured to transmit a second ultrasound signal to propagate through the anatomic structure across the third portion and the fourth portion and to receive a second set of ultrasound signals that have propagated through the anatomic structure across the third portion and the fourth portion and are indicative of a second fluid flow parameter of the biological fluid in the anatomic structure.

73. The device of claim 21, wherein the connection apparatus includes a port in electrical communication with the electronics unit housed in the connection apparatus and operable to electrically couple to a remote power supply housed in a remote device via at least one of a cable, wire, or cord, wherein the remote device is located in another location different than and at a distance from the anatomic structure.

74. The device of claim 73, wherein the port is in data communication with the electronics unit housed in the connection apparatus and operable to interface the electronics unit to a data processing unit disposed in the remote device via the at least one of the cable, wire, or cord to output the data from the electronics unit of the sensor device to the data processing unit of the remote device through the port via the at least one of the cable, wire, or cord.

75. The device of claim 74, wherein the implantable medical device is configured to wirelessly transmit the data by a secondary transmission device comprising a wireless transmitter or transceiver to the external processor.

76-87. (canceled)
